(19) United States
(12) Patent Application Publication
(10) Pub. No.: US 2025/0332414 A1
(43) Pub. Date: Oct. 30, 2025

(54) Wearable Non-Invasive Central Nervous System Neuromodulator and Methods for Using Same

(71) Applicant: SpineX Inc., Northridge, CA (US)

(72) Inventor: Parag Gad, Northridge, CA (US)

(21) Appl. No.: 18/702,756

(22) PCT Filed: Nov. 4, 2022
(86) PCT No.: PCT/US2022/048974

§ 371 Date: Apr. 18, 2024

Related U.S. Application Data
(60) Provisional application No. 63/276,298, filed on Nov. 5, 2021.
(60) Provisional application No. 63/312,607, filed on Feb. 22, 2022.
(60) Provisional application No. 63/336,914, filed on Apr. 29, 2022.

(51) Int. Cl. A61N 1/36

(52) U.S. Cl. CPC A61N 1/36025; A61N 1/36003; A61N 1/36034

(57) ABSTRACT

Aspects of the present disclosure include neuromodulating the central nervous system of a subject (e.g., a subject having delayed or abnormal brain development). Methods according to certain embodiments include applying electrical stimulation to the spinal cord of a subject in a manner sufficient to maintain voluntary control of physical activity during and after the electrical stimulation. Methods according to the certain embodiments include applying electrical stimulation to the spinal cord of a subject in a manner to induce neuroplasticity of the brain and spinal cord neural network of the subject. In some instances, neuromodulation provides for acceleration of developmental milestones, initially delayed due to delayed or abnormal development. Systems having one or more electrodes (e.g., spring loaded electrodes) configured for applying electrical stimulation to the spinal cord of the subject suitable for practicing the subject methods are also described.

### CROSS-REFERENCE TO RELATED APPLICATIONS

This application claims priority pursuant to 35 U.S.C. § 119(e) to the filing date of U.S. Provisional Patent Application Ser. No. 63/276,298 filed Nov. 5, 2021; U.S. Provisional Patent Application Ser. No. 63/312,607 filed Feb. 22, 2022 and U.S. Provisional Patent Application Ser. No. 63/336,914 filed Apr. 29, 2022, the disclosures of which applications are incorporated herein by reference in their entirety.

### INTRODUCTION

The spinal networks play a pivotal role in the control of the movements of the limbs, breathing, speech, eating, vision as well as other vital bodily functions including cardiovascular, bladder and/or bowel and sexual function. Injuries and neurodegenerative diseases can have a devastating impact on the quality of lives of the many people which suffer from these each year. Neurodegenerative conditions and diseases such as stroke, Parkinson's disease, Huntington's disease, Alzheimer's disease, amyotrophic lateral sclerosis (ALS), primary lateral sclerosis (PLS), dystonia, cerebral palsy as well as serious spinal cord injury such as from a sports injury or a traumatic accident can cause partial or total loss of cortical and voluntary sensation and autonomic function and motor function. Abnormal or delayed brain development, such as in children which suffer brain damage due to non-traumatic conditions within a few years (e.g., ˜2-3 years) after birth can affect a subject's ability to control muscles. The activity of spinal cord networks can be regulated supraspinally and by peripheral sensory input. For example, the connections between the brain and spinal cord can be enabled by electrical stimulation of the lumbosacral and cervical segments as well as the brainstem.

### SUMMARY

Aspects of the present disclosure include neuromodulating the central nervous system of a subject (e.g., a subject having delayed or abnormal brain development). Methods according to certain embodiments include applying electrical stimulation to the spinal cord of a subject in a manner sufficient to maintain voluntary control of physical activity during and after the electrical stimulation. Methods according to the embodiments include applying electrical stimulation to the spinal cord of a subject in a manner to neuromodulate it without inducing any responses. Methods according to the embodiments include applying electrical stimulation to the spinal cord of a subject in a manner to induce neuroplasticity of the brain and spinal cord neural network of the subject. In some instances, the electrical stimulation is applied to the spinal cord of the subject to retrain the spinal neural network of the central nervous system of the subject, such as a subject having a spinal cord injury, an ischemic brain injury or a neurodegenerative condition. In some instances, neuromodulation provides for acceleration of developmental milestones, initially delayed due to delayed or abnormal development. Systems having one or more electrodes (e.g., spring loaded electrodes) configured for applying electrical stimulation to the spinal cord of the subject suitable for practicing the subject methods are also described.

In embodiments, electrical stimulation is applied to the spinal cord of the subject. In some embodiments, the subject is a subject that has a condition selected from a spinal cord injury, an ischemic brain injury or a neurodegenerative condition. In some instances, the subject has an ischemic brain injury from a stroke or acute trauma. In some instances, the subject has a neurodegenerative condition such as a stroke, spinal cord injury, Parkinson's disease, Huntington's disease, Alzheimer's disease, amyotrophic lateral sclerosis (ALS), primary lateral sclerosis (PLS), dystonia, hemispherictomy, transverse myelitis, conus medularis injury (lower motor neuron injury), spina bifida, autism, hemispherectomy or cerebral palsy. In certain instances, the subject has a naturally occurring condition that results in degeneration of the central nervous system such as aging, post-partum, inactivity and post-surgical care.

In some embodiments, the subject is a subject that has or exhibits delayed or abnormal brain development. In some instances, the subject is a subject that has suffered non-traumatic brain damage. In certain instances, the subject suffered the non-traumatic brain damage within about 2-3 years from birth. In certain embodiments, the subject is diagnosed with Fragile X syndrome, Trisomy 21, a chromosomal abnormality, tuberous sclerosis, neurofibromatosis, phenylketonuria, a myopathy, Hydrocephalus, Lissencephaly, spina bifida, autism spectrum disorder, fetal alcohol syndrome, Landau Kleffner syndrome or cerebral palsy. In certain instances, the subject exhibits symptoms of or is diagnosed with cerebral palsy. In some embodiments, the applied electrical stimulation is sufficient to integrate and reconnect the brain to the spinal cord. In some instances, applying electrical stimulation to the spinal cord of the subject is sufficient to increase ascending neural signals to the brain of the subject, such as increasing cortical and voluntary sensation. In some instances, the applied electrical stimulation excites neurons in the brain. In other instances, the applied electrical stimulation inhibits neurons in the brain. In certain instances, the applied electrical stimulation simultaneously excites some neurons in the brain and inhibits some neurons in the brain. In some instances, the applied electrical stimulation reconnects the spinal neural network with the brain of the subject. In certain instances, applying electrical stimulation to the spinal cord of the subject retrains the spinal neural network of the central nervous system of the subject.

In some instances, the applied electrical stimulation enhances voluntary control of physical activity by the subject. In some embodiments, the electrical stimulation is applied to the spinal cord of the subject in a manner sufficient to maintain voluntary control of one or more of physical motor function, sensory function, vestibular function, cognitive function, autonomic function and sleep activity. In some embodiments, the electrical stimulation is applied to the spinal cord of the subject in a manner sufficient to maintain voluntary control of one or more of anxiety, depression and mood. In some instances, voluntary control of physical activity is maintained by the subject after cessation of the electrical stimulation. In some instances, the electrical stimulation is applied to the spinal cord of the subject to improve voluntary control of joints and muscles of lower extremity, upper extremity, head, neck, facial muscles, sphincters (bladder/bowel), pelvic floor, abdominal, diaphragm, throat muscles, autonomic control of organs, including bladder bowel cardiovascular sexual breathing functions. In some instances, the electrical stimulation is applied to the spinal cord of the subject to improve sensation of joints and muscles of lower extremity, upper extremity, head, neck, facial muscles, sphincters (bladder/bowel), pelvic floor, abdominal, diaphragm, throat muscles, autonomic control of organs including bladder bowel cardiovascular sexual breathing functions.

In some embodiments, the electrical stimulation is applied at a frequency and amplitude that is sufficient to activate one or more of the sensory neurons and the interneurons of the spinal cord neural network. In some instances, the electrical stimulation is applied at a frequency and amplitude sufficient to activate sensory neurons of the spinal cord neural network. In some instances, the electrical stimulation is applied at a frequency and amplitude sufficient to activate interneurons of the spinal cord neural network. In certain embodiments, neuromodulation does not directly activate the motor neurons of the spinal cord neural network. In some instances, the electrical stimulation is applied at a frequency and amplitude which activates interneurons of the spinal cord sufficient to facilitate signal conduction to motor neurons.

In some embodiments, the signal conduction to the motor neurons provides for voluntary muscle control by the subject. The voluntary muscle control may be one or more of activating one or more muscle groups, inhibiting activity by one or more muscle groups and having no impact on one or more muscle groups. In some instances, the voluntary muscle control includes the absence or reduced presence of spasticity exhibited by the subject. In some instances, the voluntary muscle control includes the absence or reduced presence of one or more of reflexes, floppiness or involuntary movements exhibited by the subject. In some instances, the voluntary muscle control includes the absence or reduced presence of co-contraction of antagonistic muscle activity exhibited by the subject.

In some embodiments, the electrical stimulation is applied in a manner sufficient to enable and learn a non-patterned, non-repetitive, stochastic motor response by the subject. In some instances, the electrical stimulation enables voluntary motor initiation response by the subject. In some instances, the electrical stimulation enables voluntary control of trunk alignment by the subject. In some instances, the electrical stimulation enables voluntary control of posture by the subject. In some instances, the electrical stimulation enables voluntary control during dynamic standing and stepping by the subject. In some instances, the electrical stimulation enables voluntary control of the center of mass by the subject. In certain instances, voluntary control of the center of mass includes maintaining the center of mass of the subject over a base of support. In certain embodiments, the electrical stimulation enables voluntary control by the subject sufficient to perform one or more of head control, stepping, climbing, upright sitting, shifting weight, control movement or alignment of the trunk, dynamic standing with postural or weight adjustment, transition from sitting to standing, transition from stand to walk, walk to run, increasing and decreasing speed of walking, transition from standing to sitting, crawling, proning, rolling, nodding and gesturing.

In some embodiments, the neuromodulation includes applying the electrical stimulation in a manner sufficient to provide for identifying and maintaining midline orientation by the subject. In some instances, the electrical stimulation provides for identifying midline orientation with bilateral hand and arm activities. For example, the bilateral hand and arm activities may include clapping or jumping jacks. In some instances, the electrical stimulation provides for maintaining weight bearing standing by the subject. For instance, weight bearing standing with heels on the ground may be maintained by the subject. In other instances, the electrical stimulation provides for maintaining weight bearing sitting balance by the subject. For instance, weight bearing sitting balance with head over ischial tuberosities may be maintained by the subject. In certain instances, the electrical stimulation provides for maintaining a predetermined balance and posture by the subject.

In some embodiments, the method includes maintaining the head in an upright position with the eyes parallel to the horizontal plane by the subject for appropriate visual input. In some instances, the method includes maintaining by the subject the head, trunk, pelvis and ischial tuberosities in alignment with the center of mass directly over the ischial tuberosities. In some instances, the method includes maintaining the hands and arms free to explore and interact with a surrounding space and further increase proprioceptive information from an upper extremity by the subject. In some instances, the method includes generating by the subject one or more of weight shifts, postural adjustments, external support and changes in alignment by movement of the hip and pelvis. In certain instances, the subject does not move the shoulders and ankles.

In some embodiments, neuromodulation according to methods of the present disclosure increase processing of proprioception in the brain and spinal cord. In some instances, neuromodulation as described herein increases processing of descending voluntary signals from the brain to the spinal cord of the subject. In some instances, increasing proprioception in the brain and spinal cord of the subject is sufficient to facilitate sense of touch by the subject. In some instances, increasing proprioception in the brain and spinal cord of the subject is sufficient to facilitate or improve judgement of distance by the subject. In some instances, increasing proprioception in the brain and spinal cord of the subject is sufficient to facilitate or improve judgement of object size by the subject. In some embodiments, neuromodulation increases proprioception in the brain and spinal cord of the subject sufficient to improve visual tracking by the subject. In one example, neuromodulation according to embodiments improves peripheral visual tracking by the subject. In another example, neuromodulation according to embodiments improves cross-midline visual tracking by the subject. In some embodiments, neuromodulation increases proprioception in the brain and spinal cord of the subject in a manner sufficient to change cortical visual impairment of the subject. In some instances, increasing proprioception in the brain and spinal cord of the subject is sufficient to improve visual focus of the subject.

In some embodiments, electrical stimulation according to methods of the present disclosure increase proprioception in the brain and spinal cord of the subject sufficient to facilitate or improve judgement of falling by the subject. In some instances, increasing proprioception in the brain and spinal cord of the subject is sufficient to prevent involuntary falling by the subject. In some embodiments, the electrical stimulation increases proprioception in the brain and spinal cord of the subject sufficient to provide for voluntary control of two or more of the head, hands and arm, trunk, and legs in a synchronized manner. For example, the voluntary control includes aligning two or more of the head, hands and arms, trunk, and legs. In some instances, the voluntary control includes maintaining two or more of the head, hands and arms, trunk, and legs in alignment with the center of mass directly over the base of support while walking.

In some embodiments, neuromodulation includes applying the electrical stimulation in a manner sufficient to increase self-motivation, excitement and engagement in activities by the subject. In some instances, neuromodulation increases self-initiated communication, such as non-verbal communication including but not limited to one or more of gestures, eye tracking, eye movement, head nodding, smiling, crying and laughing. In some instances, neuromodulation increases verbal communication by the subject. In some embodiments, the method includes providing one or more of verbal and tactile queues to the subject. In some instances, the verbal or tactile queues are sufficient to allow the subject to voluntarily correct an error. In certain instances, physical assistance is provided to the subject only after the subject has committed an error. For instance, assistance is not provided during or prior to the error being committed.

In some embodiments, increasing proprioception in the brain and spinal cord of the subject is sufficient to increase spatial recognition by the subject. In some instances, the spatial recognition includes informing the subject as to where one or more parts of the body are in space. In some instances, neuromodulation increases proprioception in the brain and spinal cord of the subject in when the subject is in prone position, the center of mass is in the pelvis with the ground reaction forces acting on the anterior surface of the body. In some instances, neuromodulation increases proprioception in the brain and spinal cord of the subject when the subject is in sitting position, the center of mass is directly over the ischial tuberosities. In some instances, neuromodulation increases proprioception in the brain and spinal cord of the subject when the subject is in quadruped position, the center of mass is in between the knees and hands and the ground reaction forces are at the heels of the hands, the knees and the feet. In some instances, neuromodulation increases proprioception in the brain and spinal cord of the subject when the subject is standing on a two-leg position, the center of mass is directly in between the two feet, over the heels. In some instances, neuromodulation increases proprioception in the brain and spinal cord of the subject when the subject is on a one-leg position, the center of mass is directly over the heel in contact with the ground.

In some embodiments, neuromodulation includes applying the electrical stimulation at frequency and amplitude sufficient to improve intellectual disabilities of the subject. In some instances, neuromodulation reduces a long term complication in the subject, such as one or more of contractures, joint displacement, depression, social anxiety, heart and lung diseases, osteoarthritis and osteoporosis.

In some embodiments, the subject methods include applying the electrical stimulation to the spinal cord of the subject to improve vision in the subject, such as one or more of near sightedness, far sightedness, peripheral vision and visual acuity. In some instances, the applied electrical stimulation improves the sense of smell in the subject. In other instances, the applied electrical stimulation improves the sense of hearing by the subject. In certain instances, the applied electrical stimulation improves voice modulation by the subject, such as improving one or more of the ability to vocalize, articulation, speaking softly, speaking loudly, and duration of voice modulation by the subject.

In some instances, methods include applying electrical stimulation to the spinal cord of the subject to improve the ability to control one or more of swallowing, biting, sipping, movement of the lower jaw, movement of the tongue by the subject. In some instances, the applied electrical stimulation improves the control of facial muscles by the subject, such as improving smiling by the subject. In other instances, applying electrical stimulation improves the sense of taste, movement of the eyeballs or movement of the head and neck by the subject.

In certain embodiments, applying electrical stimulation to the spinal cord of the subject is sufficient to improve sleep by the subject, such as the ability to fall asleep faster, ability to sleep longer without waking up at night and ability to go back to sleep after waking up. In some instances, the applied electrical stimulation reduces or normalizes seizure activity by the subject. In other instances, the applied electrical stimulation reduces or normalizes the resting state of the nervous system of the subject.

In some embodiments, methods include applying the electrical stimulation to the spinal cord of the subject to treat anxiety or depression in the subject. In other embodiments, methods include applying the electrical stimulation to the spinal cord of the subject to improve vestibular function in the subject such as improving vertigo, dizziness, visual disturbance, and imbalance. In some embodiments, the applied electrical stimulation improves bladder function in the subject such as by increasing bladder capacity, increasing sensation of bladder fullness, reducing urinary incontinence, increasing voluntary control to hold, improving ability to void voluntarily, reducing the use of catheters to empty bladder. In other embodiments, applying electrical stimulation to the spinal cord of the subject improves bowel function in the subject such as by increasing sensation of bowel fullness, reducing fecal incontinence, increasing voluntary control to hold and improving the ability to defecate voluntarily. In other embodiments, applying electrical stimulation to the spinal cord of the subject improves sexual function in the subject such as by improving sensation of urogenital organs, returning the ability to have an erection, increasing lubrication, increasing sensation during erection and penetration, increasing ability for voluntary penetration, increasing ability to sustain erection for longer periods of time and increasing degree of orgasm at climax. In certain instances, the applied electrical stimulation increases sperm count, sperm mortality and vitality by the subject.

Aspects of the present disclosure according to certain embodiments include a method of neuromodulation in a subject having a spinal cord injury. In some instances, the method includes applying electrical stimulation to the spinal cord of the subject acutely after the spinal cord injury in a manner sufficient to induce a plastic change in one or more of the brain and spinal cord. In some embodiments, the electrical stimulation is applied to the spinal cord of the subject 6 months or less after the spinal cord injury. In some embodiments, the electrical stimulation is applied to the spinal cord of the subject 3 months or less after the spinal cord injury. In certain embodiments, the electrical stimulation is applied to the spinal cord of the subject 6 weeks or less after the spinal cord injury. In some instances, the electrical stimulation is applied to the spinal cord of the subject before post-injury innervation. In some instances, the electrical stimulation is applied to the spinal cord of the subject before post-injury hyperinnervation. In certain instances, the electrical stimulation is applied to the spinal cord of the subject during post-injury spinal shock.

In some embodiments, the electrical stimulation is applied to the spinal cord of the subject in a manner sufficient to prevent aberrant connections in the brain and spinal cord of the subject. In some embodiments, the neuromodulation is sufficient to prevent aberrant connections in the brain and spinal cord of the subject during post-injury spinal shock. In some embodiments, the electrical stimulation is applied to the spinal cord of the subject so as to reduce or prevent scar tissue formation at the site of the spinal cord injury. In some embodiments, the electrical stimulation is applied to the spinal cord of the subject in a manner sufficient to increase blood flow to the site of the spinal cord injury. In other instances, the electrical stimulation increases the blood flow to a site along the spinal cord that is above the spinal cord injury and/or to a site along the brain that is above the spinal cord injury. In other instances, the electrical stimulation increases blood flow to a site along the spinal cord that is below the spinal cord injury.

In some embodiments, the electrical stimulation is applied to the spinal cord of the subject in a manner sufficient to delay or prevent detrusor overactivity in the subject. In some instances, the electrical stimulation delays or prevents detrusor overactivity in the subject during post-injury spinal shock. In some instances, the electrical stimulation reduces spasticity of the detrusor and urethral sphincter. In certain embodiments, the neuromodulation increases voluntary control of the urethral sphincter in the subject to allow contraction and relaxation of the muscle based on whether the subject intends to store urine or void urine. In some instances, the subject is capable of one or more of storing urine in the bladder and voluntarily voiding the urine from the bladder during electrical stimulation. In other instances, the subject is capable of one or more of storing urine in the bladder and voluntarily voiding the urine from the bladder in the absence of active electrical stimulation. In other instances, the subject is capable of one or more of voluntarily contracting the detrusor and simultaneously relaxing the urethral sphincter in the absence of active electrical stimulation. In certain embodiments, neuromodulation is sufficient to increase sense by the subject of bladder fullness. In other embodiments, neuromodulation is sufficient to increase bladder capacity of the subject.

In some embodiments, the electrical stimulation is applied to the spinal cord of the subject in a manner sufficient to facilitate voluntary delayed voiding contraction in the absence of active stimulation after neuroplasticity is induced by the spinal neuromodulation. In some instances, voiding contraction is delayed by an applied voluntary increase in urethral pressure by the subject in the absence of active stimulation after neuroplasticity is induced by the spinal neuromodulation. In certain instances, the voluntary increase in urethral pressure is applied in a sustained manner in the absence of active stimulation after neuroplasticity is induced by the spinal neuromodulation. In certain instances, the voluntary increase in urethral pressure is not applied in a spastic manner in the absence of active stimulation after neuroplasticity is induced by the spinal neuromodulation. In some embodiments, the electrical stimulation is applied to the spinal cord of the subject in a manner sufficient to facilitate voluntary detrusor contraction in the absence of active stimulation after neuroplasticity is induced by the spinal neuromodulation. In some embodiments, the electrical stimulation is applied to the spinal cord of the subject in a manner sufficient to facilitate a decrease in urethral pressure in response to voluntary detrusor contraction in the absence of active stimulation after neuroplasticity is induced by the spinal neuromodulation. In some instances, the frequency of voluntary voids increases in the absence of active stimulation after neuroplasticity is induced by the spinal neuromodulation. In some instances, the volume of voluntary voids increases in the absence of active stimulation after neuroplasticity is induced by the spinal neuromodulation. In some instances, the number of catheters used decreases in the absence of active stimulation after neuroplasticity is induced by the spinal neuromodulation.

In some embodiments, the electrical stimulation is applied to the spinal cord of the subject in a manner sufficient to increase one or more of voluntary initiation and voluntary completion of bowel movement by the subject in the absence of active stimulation after neuroplasticity is induced by the spinal neuromodulation. In some instances, spinal neuromodulation results in neuroplasticity of the brain and/or spinal cord sufficient to increase sense by the subject of bowel fullness in the absence of active stimulation. In some instances, the electrical stimulation is applied to the spinal cord of the subject resulting in neuroplasticity of the brain and/or spinal cord sufficient to facilitate voluntary contractions of one or more of the anus, rectum and other bowel sections in the absence of active stimulation.

In certain embodiments, the electrical stimulation is applied to the spinal cord of the subject resulting in neuroplasticity of the brain and/or spinal cord in a manner sufficient to increase one or more voluntary sexual function by the subject in the absence of active stimulation. In some instances, the electrical stimulation is applied to the spinal cord of the subject resulting in neuroplasticity of the brain and/or spinal cord in a manner sufficient to facilitate voluntary generation of psychogenic erection by the subject in the absence of active stimulation. In some instances, the electrical stimulation is applied to the spinal cord of the subject resulting in neuroplasticity of the brain and/or spinal cord in a manner sufficient to facilitate voluntary generation of reflex erection by the subject in the absence of active stimulation. In certain instances, the electrical stimulation is applied to the spinal cord of the subject resulting in neuroplasticity of the brain and/or spinal cord in a manner sufficient to facilitate voluntary ejaculation by the subject in the absence of active stimulation. In some embodiments, the electrical stimulation is applied to the spinal cord of the subject resulting in neuroplasticity of the brain and/or spinal cord in a manner sufficient to facilitate performance of sexual intercourse by the subject in the absence of active stimulation. In certain instances, the electrical stimulation is applied to the spinal cord of the subject resulting in neuroplasticity of the brain and/or spinal cord in a manner sufficient to increase or improve sense of sexual function by the subject in the absence of active stimulation.

In practicing methods according to embodiments, electrical stimulation is applied to the spinal cord of the subject, where in certain instances, the electrical stimulation is applied having a waveform selected from the group of: one or more a trapezoidal monophasic waveform and a trapezoidal biphasic waveform; one or more of a triangular monophasic waveform and triangular biphasic waveform; an asymmetrical biphasic waveform; a double monophasic waveform; and a monophasic waveform. In some instances, the one or more applied waveform further includes a DC offset. In certain instances, the DC offset is an applied voltage that is sufficient to compensate for each applied electrical stimulation pulse. In some embodiments, methods include applying the electrical stimulation from two or more channels of a transcutaneous or epidural electrical spinal cord stimulator. In some instances, each channel of the transcutaneous or epidural electrical spinal cord stimulator independently applies a different waveform of electrical stimulation. In certain instances, the applied electrical stimulation includes a plurality of different waveforms from the same electrode, where in some embodiments each waveform is applied sequentially and in other embodiments waveforms are applied simultaneously.

In some embodiments, each waveform has a high frequency component and a low frequency component. In some instances, the high frequency component has a frequency of from 1 KHz to 25 KHz, such as from 5 KHz to 15 KHz, including a high frequency component of about 10 KHz. In some instances, the low frequency component has a frequency of from 1 Hz to 500 Hz, such as from 50 Hz to 250 Hz, including a low frequency component of about 100 Hz. In some embodiments, each applied waveform has a high frequency component and a low frequency component where the high frequency component provides an analgesic effect and the low frequency component provides for the neuromodulation of the nervous system as described herein. In certain instances, the high frequency component is sufficient to provide an analgesic effect to the skin of the subject and the low frequency component is sufficient to tune spinal cord neurons to achieve the desired functional goals (i.e., the functional goals of neuromodulation)

In some embodiments, the electrical stimulation is applied to the spinal cord of the subject at a pulse frequency of 5 Hz or more, such as at a pulse frequency of 25 Hz or more and including about a pulse frequency of about 30 Hz. In some embodiments, the electrical stimulation is applied to the spinal cord of the subject with a pulse amplitude of from 1 mA to 500 mA, such as from 50 mA to 200 mA, including a pulse amplitude of about 100 mA. When a DC offset is applied, the DC offset amplitude may be from 0.1 mA to 10 mA, such as from 0.5 mA to 2.5 mA, including a DC offset amplitude of about 1.5 mA. The applied DC offset may be a pulsed DC offset or a continuously applied DC offset.

The electrical stimulation may be applied to the spinal cord of the subject for 1 hour or more, such as for 8 to 12 hours per day. Depending on the condition being treated, methods may include applying the electrical stimulation to the subject 2 to 5 days per week.

In some instances, methods further include applying one or more of magnetic stimulation and mechanical stimulation to the spine of the subject. In some instances, methods include applying magnetic stimulation sequentially with the electrical stimulation. In other instances, methods include applying magnetic stimulation simultaneously with the electrical stimulation. In some instances, methods include applying mechanical stimulation sequentially with the electrical stimulation. In other instances, methods include applying mechanical stimulation simultaneously with the electrical stimulation. In yet other instances, methods including applying magnetic and mechanical stimulation sequentially with the electrical stimulation. In still other instances, methods include applying magnetic and mechanical stimulation simultaneously with the electrical stimulation.

Aspects of the disclosure also include systems for practicing the subject methods. Systems according to certain embodiments include an electrical stimulator that is configured to apply electrical stimulation to the spinal cord of a subject in a manner sufficient to maintain voluntary control of physical activity during the electrical stimulation. In some embodiments, systems include a wearable electrical stimulator device. In some instances, the wearable device includes a single use battery. In other instances, the wearable device includes a rechargeable battery. In certain instances, the wearable device is disposable. In some embodiments, the wearable device is configured to route wires under the clothing of the subject. In some instances, the electrical stimulator is integrated into clothing or furniture (e.g., chair) or some other device which positions the electrical stimulator at a location along the spinal cord of the subject. In certain embodiments, the electrical stimulator is integrated into or operationally associated with one or more of a powered exoskeleton device, a powered or active orthosis, a passive orthosis, a wearable orthosis, a soft exoskeleton device, a hip orthosis, a knee orthosis, a head orthosis, an ankle orthosis, a body weight support device, a stand frame, a wheelchair, a set of crutches and a walker.

In certain embodiments, the electrical stimulator includes a set of spring-loaded electrodes that are configured to ensure hydrogel contact between the electrodes with the skin of the subject. For example, the electrical stimulator may be integrated into a belt or harness with worn springs. In some instances, the springs of the electrical stimulator device is configured to provide mechanical and vibrotactile stimulation.

In some embodiments, the electrical stimulator is configured to apply electrical stimulation to the spinal cord of the subject having a waveform selected from the group of: one or more a trapezoidal monophasic waveform and a trapezoidal biphasic waveform; one or more of a triangular monophasic waveform and triangular biphasic waveform; an asymmetrical biphasic waveform; a double monophasic waveform; and a monophasic waveform. In some instances, the electrical stimulator is configured to apply one or more of the waveforms with a DC offset. In certain instances, the DC offset is an applied voltage that is sufficient to compensate for each applied electrical stimulation pulse. In some embodiments, systems include a transcutaneous or epidural electrical spinal cord stimulator that is configured to apply electrical stimulation from two or more channels. In some instances, each channel of the transcutaneous or epidural electrical spinal cord stimulator independently applies a different waveform of electrical stimulation. In certain instances, the transcutaneous or epidural electrical spinal cord stimulator applies a plurality of different waveforms from the same electrode, where in some embodiments each waveform is applied sequentially and in other embodiments waveforms are applied simultaneously.

In some embodiments, the electrical stimulator is configured to apply a waveform that has a high frequency component and a low frequency component. In some instances, the high frequency component has a frequency of from 1 KHz to 25 KHz, such as from 5 KHz to 15 KHz, including a high frequency component of about 10 KHz. In some instances, the low frequency component has a frequency of from 1 Hz to 500 Hz, such as from 50 Hz to 250 Hz, including a low frequency component of about 100 Hz. In some embodiments, the electrical stimulator is configured to apply a waveform that has a high frequency component and a low frequency component where the high frequency component provides an analgesic effect and the low frequency component provides for the neuromodulation of the nervous system as described herein. In certain instances, the high frequency component applied by the electrical stimulator is sufficient to provide an analgesic effect to the skin of the subject and the low frequency component is sufficient to tune spinal cord neurons to achieve the desired functional goals (i.e., the functional goals of neuromodulation)

In some embodiments, the electrical stimulator is configured to apply electrical stimulation to the spinal cord of the subject having a pulse frequency of 5 Hz or more, such as at a pulse frequency of 25 Hz or more and including about a pulse frequency of about 30 Hz. In some embodiments, the electrical stimulator is configured to apply electrical stimulation to the spinal cord of the subject having a pulse amplitude of from 1 mA to 500 mA, such as from 50 mA to 200 mA, including a pulse amplitude of about 100 mA. When a DC offset is applied, the DC offset amplitude may be from 0.1 mA to 10 mA, such as from 0.5 mA to 2.5 mA, including a DC offset amplitude of about 1.5 mA. In some instances, the electrical stimulator is configured to apply the DC offset as a pulsed DC offset. In other instances, the electrical stimulator is configured to apply the DC offset continuously.

### BRIEF DESCRIPTION OF THE FIGURE

FIG. 1 depicts a trapezoidal waveform pattern for transcutaneous and/or epidural stimulation according to certain embodiments.

FIG. 2 depicts a triangular waveform pattern for transcutaneous and/or epidural stimulation according to certain embodiments.

FIG. 3 depicts an asymmetrical biphasic waveform pattern for transcutaneous and/or epidural stimulation according to certain embodiments.

FIG. 4 depicts a double monophasic waveform pattern for transcutaneous and/or epidural stimulation according to certain embodiments.

FIG. 5 depicts a monophasic waveform with opposite DC offset to balance the applied voltage from the monophasic waveform for transcutaneous and/or epidural stimulation according to certain embodiments.

FIGS. 6A and 6B depict an electrode holder according to certain embodiments.

FIGS. 7A and 7B depict subsystems of a transcutaneous/epidural electrical stimulator according to certain embodiments.

FIG. 8 depicts applying electrical stimulation which provides for voluntary muscle control and induced neuroplasticity (e.g., in a subject having delayed or abnormal development) according to certain embodiments.

### DETAILED DESCRIPTION

Aspects of the present disclosure include neuromodulating the central nervous system of a subject (e.g., a subject having delayed or abnormal brain development). Methods according to certain embodiments include applying electrical stimulation to the spinal cord of a subject in a manner sufficient to maintain voluntary control of physical activity during and after the electrical stimulation. Methods according to the embodiments include applying electrical stimulation to the spinal cord of a subject in a manner to neuromodulate it without inducing any responses. Methods according to the embodiments include applying electrical stimulation to the spinal cord of a subject in a manner to induce neuroplasticity of the brain and spinal cord neural network of the subject. In some instances, the electrical stimulation is applied to the spinal cord of the subject to retrain the spinal neural network of the central nervous system of the subject, such as a subject having a spinal cord injury, an ischemic brain injury or a neurodegenerative condition. In some instances, neuromodulation provides for acceleration of developmental milestones, initially delayed due to delayed or abnormal development. Systems having one or more electrodes (e.g., spring loaded electrodes) configured for applying electrical stimulation to the spinal cord of the subject suitable for practicing the subject methods are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, the present disclosure provides methods for neuromodulating the central nervous system of a subject by applying electrical stimulation to the spinal cord of a subject. In further describing embodiments of the disclosure, methods for applying electrical stimulation in a manner sufficient to maintain voluntary control of physical activity during electrical stimulation is first described in greater detail. Next, systems including an electrical stimulator (e.g., a transcutaneous or epidural electrical spinal cord stimulator) are described. Wearable devices and systems integrating the subject electrical stimulators are also provided.

### Methods for Neuromodulating the Central Nervous System of a Subject

Aspects of the present disclosure include neuromodulating the central nervous system of a subject. Methods according to certain embodiments include applying electrical stimulation to the spinal cord of a subject in a manner sufficient to maintain voluntary or automatic control of physical or autonomic activity during after cessation of the electrical stimulation. In embodiments, the central nervous system of the subject is neuromodulated (i.e., excited and/or inhibited) to enable retraining of the spinal neural networks, to reconnect the spinal neural networks with the brain, enhance descending voluntary control and to increase ascending neural information to the brain. In some embodiments, neuromodulation according to embodiments is sufficient to induce neuroplasticity of the brain and spinal cord neural network of the subject. In certain embodiments, neuromodulation provides for acceleration of developmental milestones, initially delayed due to delayed or abnormal development. In some embodiments, neuromodulation provides for voluntary control of one or more muscle groups, voluntary control to inhibit one or more muscle groups, and voluntary control to have no impact on one or more other muscles groups. In certain embodiments, aspects of the present disclosure include neuromodulation in a subject having a spinal cord injury. In some instances, the neuromodulation is applied acutely after the spinal cord injury, such as 6 months or less after the spinal cord injury, such as 3 months or less and including 6 weeks or less after the spinal cord injury.

In practicing the subject methods according to certain embodiments, the spinal cord neural networks include three components: the sensory neurons, the interneurons and the motor neurons. Neuromodulation as described herein is applied over the dorsal surface of the spinal neural network to the sensory neurons at a frequency and amplitude which does not directly activate the motor neurons (e.g., at a sub-motor threshold) As described below, in some instances the applied electrical stimulation is sufficient to only activate the sensory neurons and interneurons. In some embodiments, the frequency and amplitude of the applied electrical stimulation is not sufficient to penetrate motor neurons (e.g., the ventral component of the spinal neural network). For instance, neuromodulation does not bypass the interneurons to directly activate motor neurons, where bypassing the interneurons to directly activate motor neurons causes involuntary motor responses and not voluntary control by the brain.

In some embodiments, neuromodulation includes activating only the sensory and interneurons to prime the spinal neural network such that when the spinal cord receives commands from the brain (e.g., voluntary control) and corresponding signals from the periphery (sensory/proprioceptive information), the interneurons can send the appropriate signals to the motor neurons. In certain instances, activating only the sensory and interneurons (i.e., without directly activating the motor neurons) is sufficient to provide for voluntary control of one or more muscle groups, provide for voluntary control to inhibit one or more muscle groups, and provide for voluntary control to have no impact on one or more other muscles groups.

In certain embodiments, without neuromodulation of the spinal neural network according to the present disclosure a dysfunctional neural network (e.g., caused by abnormal or delayed development or injury), the interneurons are not able to translate information (voluntary and proprioception) over to the motor neurons. In some instances, synchronization of voluntary, proprioception and neuromodulation is sufficient to provide for voluntary muscle control as described in greater detail below.

As described herein, electrical stimulation is applied to the spinal cord of a subject in need thereof. In some instances, the subjects are humans. The methods may be applied to human subjects of both genders and at any stage of development (e.g., neonate, infant, juvenile, adolescent, adult, geriatric, etc.), where in certain embodiments the human subject is a juvenile, adolescent or adult.

In some instances, subjects of the present disclosure include but are not limited to subjects that has a condition selected from a spinal cord injury, an ischemic brain injury or a neurodegenerative condition. In some instances, the subject has an ischemic brain injury from a stroke or acute trauma. In some instances, the subject has a neurodegenerative condition such as a stroke, spinal cord injury, Parkinson's disease, Huntington's disease, Alzheimer's disease, amyotrophic lateral sclerosis (ALS), primary lateral sclerosis (PLS), dystonia, hemispherictomy, transverse myelitis, conus medularis injury (lower motor neuron injury), spina bifida, autism, hemispherectomy or cerebral palsy. In certain instances, the subject has a naturally occurring condition that results in degeneration of the central nervous system such as aging, post-partum, inactivity and post-surgical care.

In some embodiments, the subject is a subject that has or exhibits delayed or abnormal brain development. In some instances, the subject is a subject that has suffered non-traumatic brain damage. In certain instances, the subject suffered the non-traumatic brain damage within about 2-3 years from birth. In certain embodiments, the subject is diagnosed with Fragile X syndrome, Trisomy 21, a chromosomal abnormality, tuberous sclerosis, neurofibromatosis, phenylketonuria, a myopathy, Hydrocephalus, Lissencephaly, spina bifida, autism spectrum disorder, fetal alcohol syndrome, Landau Kleffner syndrome or cerebral palsy. In certain instances, the subject exhibits symptoms of or is diagnosed with cerebral palsy.

In some embodiments, the subject is a subject with a spinal cord injury. The term “spinal cord injury” is used herein in it conventional sense to refer to damage or trauma to the spinal cord and may include but is not limited to damages to the tissues of the spinal cord as well as the tissues and the bones (i.e., vertebrae) that surround the spinal cord. In some embodiments, neuromodulation as described herein is applied to the spinal cord of the subject acutely after the spinal cord injury. In some instances, the neuromodulation is sufficient to induce a plastic change in the brain and/or the spinal cord. Electrical stimulation may be applied for example, 6 months or less after the spinal cord injury, such as 5.5 months or less, such as 5 months or less, such as 4.5 months or less, such as 4 months or less, such as 3.5 months or less, such as 3 months or less, such as 2.5 months or less, such as 2 months or less, such as 1.5 months or less and including 1 month or less. For instance, the electrical stimulation may be applied 25 weeks or less after the spinal cord injury, such as 20 weeks or less, such as 16 weeks or less, such as 12 weeks or less, such as 8 weeks or less and including within 6 weeks or less of the spinal cord injury.

In some instances, the electrical stimulation is applied to the spinal cord of the subject before post-injury innervation, such as 1 day or more before post-injury innervation, such as 2 days or more, such as 3 days or more, such as 4 days or more, such as 5 days or more, such as 6 days or more, such as 7 days or more, such as 2 weeks or more, such as 3 weeks or more and including 4 weeks or more before post-injury innervation. In some instances, the electrical stimulation is applied to the spinal cord of the subject before post-injury hyperinnervation. In certain instances, the electrical stimulation is applied to the spinal cord of the subject during post-injury spinal shock. In some instances, the electrical stimulation is applied the entire duration of post-injury spinal shock. In other instances, the electrical stimulation is applied during intermittent time periods during the period of post-injury spinal shock. In other instances, electrical stimulation is applied for at least a time period during post-injury spinal shock and a period after post-injury spinal shock.

In some embodiments, the electrical stimulation is applied to the spinal cord of the subject in a manner sufficient to prevent aberrant connections in the brain and spinal cord of the subject. In some embodiments, the neuromodulation is sufficient to prevent aberrant connections in the brain and spinal cord of the subject during post-injury spinal shock. In some embodiments, the electrical stimulation is applied to the spinal cord of the subject so as to reduce or prevent scar tissue formation at the site of the spinal cord injury. For example, applying electrical stimulation according to methods of the present disclosure reduces scar tissue formation at the site of spinal cord injury by 1% or more as compared to scar tissue formation at the site of spinal cord injury where no electrical stimulation is applied, such as by 2% or more, such as by 3% or more, such as by 5% or more, such as by 10% or more, such as by 25% or more, such as by 50% or more, such as by 75% or more, such as by 90% or more and including by 95% or more. In certain instances, the electrical stimulation altogether prevents scar tissue formation at the site of the spinal cord injury.

In some embodiments, the electrical stimulation is applied to the spinal cord of the subject in a manner sufficient to increase blood flow to the site of the spinal cord injury. In other instances, the electrical stimulation increases the blood flow to a site along the spinal cord that is above the spinal cord injury and/or to a site along the brain that is above the spinal cord injury. In other instances, the electrical stimulation increases blood flow to a site along the spinal cord that is below the spinal cord injury. The increase in blood flow by applying the electrical stimulation may be an increase by 1% or more as compared to blood flow in the absence of the applied electrical stimulation, such as by 2% or more, such as by 3% or more, such as by 5% or more, such as by 10% or more, such as by 25% or more, such as by 50% or more, such as by 75% or more, such as by 90% or more and including by 95% or more.

Neuromodulation according to certain embodiments is sufficient to improve intellectual disabilities of the subject. In some instances, neuromodulation reduces a long term complication in the subject, such as one or more of contractures, joint displacement, depression, social anxiety, heart and lung diseases, osteoarthritis and osteoporosis.

In practicing the methods, the subject is capable for at least a duration of the applied electrical stimulation to maintain voluntary control of physical activity. By “maintain voluntary control of physical activity” is meant that the subject for at least a duration of the applied electrical stimulation has complete control of physical activity and that the applied electrical stimulation does not induce activity. In other words, the applied electrical stimulation does not bypass the spinal network that results in no brain control over the physical activity exhibited by the subject. In some embodiments, the subject maintains voluntary control of physical activity for 5% or more of the duration that electrical stimulation is applied to the spinal cord, such as for 10% or more, such as for 25% or more, such as for 50% or more, such as for 60% or more, such as for 70% or more, such as for 80% or more, such as for 90% or more, such as for 95% or more, such as by for 97% or more, such as for 99% or more and including where the subject maintains voluntary control of physical activity for 99.9% or more of the duration that electrical stimulation is applied to the spinal cord. In certain embodiments, the subject maintains voluntary control of physical activity for the entire duration (i.e., 100%) that the electrical stimulation is applied to the spinal cord. In other words, applying electrical stimulation according to the methods enables connection to the brain and activity controlled completely by the subject. In some embodiments, the voluntary control of physical activity is maintained by the subject after cessation of the electrical stimulation. In some embodiments, the voluntary control consists of acutonomic activity is maintained by the subject during and after cessation of the electrical stimulation.

In some instances, the electrical stimulation is applied at a frequency and amplitude sufficient to activate sensory neurons of the spinal cord neural network. In some instances, the electrical stimulation is applied at a frequency and amplitude sufficient to activate interneurons of the spinal cord neural network. In certain embodiments, neuromodulation does not directly activate the motor neurons of the spinal cord neural network. In some instances, the electrical stimulation is applied at a frequency and amplitude which activates interneurons of the spinal cord sufficient to facilitate signal conduction to motor neurons. In some embodiments, the signal conduction to the motor neurons provides for voluntary muscle control by the subject. The voluntary muscle control may be one or more of activating one or more muscle groups, inhibiting activity by one or more muscle groups and having no impact on one or more muscle groups.

In some instances, the voluntary muscle control includes the absence or reduced presence of spasticity exhibited by the subject. In some instances, the voluntary muscle control includes the absence or reduced presence of one or more of reflexes, floppiness or involuntary movements exhibited by the subject. In some instances, the voluntary muscle control includes the absence or reduced presence of co-contraction of anagonistic muscle activity exhibited by the subject. By “absence or reduced presence” is meant that spasticity, floppiness, involuntary movements, etc. is reduced by 5% or more as compared to the absence of the applied electrical stimulation, such as by 10% or more, such as by 25% or more, such as by 50% or more, such as by 75% or more, such as by 90% or more, such as by 95% or more, such as by 99% or more and including where the spasticity, floppiness, involuntary movements, etc. of the subject is entirely eliminated as compared to when the electrical stimulation as described herein is not applied to the subject. In some embodiments, applying an electrical stimulation increases cortical or voluntary sensation. The increase in cortical or voluntary sensation by the subject in response to the applied electrical stimulation may be 1% or more as compared to cortical or voluntary sensation in the absence of the applied electrical stimulation, such as 5% or more, such as 10% or more, such as 25% or more, such as 50% or more, such as 75% or more, such as 90% or more and including 100% or more. For example, applying the electrical stimulation may increase cortical or voluntary sensation experienced by the subject by 1.5 fold or more, such as 2 fold or more, such as 3 fold or more, such as 4 fold or more, such as 5 fold or more and including by 10 fold or more.

In some embodiments, applying an electrical stimulation as described herein is sufficient to increase neural signals. In some instances, the applied electrical stimulation increases neural signals from the brain to the spinal cord. In some instances, the applied electrical stimulation increases neural signals from the spinal cord to the muscles of the subject. In some instances, the applied electrical stimulation increases neural signals from the muscles to the spinal cord. In some instances, the applied electrical stimulation increases ascending neural signals from the spinal cord to the brain. The increase in neural signals in response to the applied electrical stimulation may be 1% or more as compared to neural signals measured in the absence of the applied electrical stimulation, such as 5% or more, such as 10% or more, such as 25% or more, such as 50% or more, such as 75% or more, such as 90% or more and including 100% or more. For example, applying the electrical stimulation may increase neural signals (e.g., ascending neural signals from the spinal cord to the brain) by 1.5 fold or more, such as 2 fold or more, such as 3 fold or more, such as 4 fold or more, such as 5 fold or more and including by 10 fold or more.

In some embodiments, the applied electrical stimulation is sufficient to excite neurons in the brain. In other embodiments, the applied electrical stimulation is sufficient to inhibit neurons in the brain. In yet other embodiments the applied electrical stimulation is sufficient to excite a subset of neurons in the brain and inhibit a subset of neurons in the brain. Depending on the electrical stimulation applied, in some instances the ratio of excited neurons to inhibited neurons in the brain may range from 1:1 to 1:100, such as from 1:1 to 1:75, such as from 1:1 to 1:50, such as from 1:1 to 1:25, such as from 1:1 to 1:10, including from 1:1 to 1:5. In other instances, the ratio of excited neurons to inhibited neurons in the brain may range from 100:1 to 1:1, such as from 75:1 to 1:1, such as from 50:1 to 1:1, such as from 25:1 to 1:1, such as from 10:1 to 1:1 and including from 5:1 to 1:1.

In some embodiments, the electrical stimulation is applied in a manner sufficient to enable a stochastic motor response by the subject. The term “stochastic motor response” is used herein in its conventional sense to refer to a motor response which is non-patterned and non-repetitive. In certain embodiments, stochastic motor response refers to a step by step difference with no two steps being identical with variations in position and angles of all the joints and the phase difference between left and right limbs. In certain embodiments, the electrical stimulation does not generate a locomotor pattern by the subject.

In some instances, the electrical stimulation is applied at a frequency and amplitude which enables voluntary motor initiation response by the subject. In some instances, the electrical stimulation enables voluntary control of trunk alignment by the subject. In some instances, the electrical stimulation enables voluntary control of posture by the subject. In some instances, the electrical stimulation enables voluntary control during dynamic standing and stepping by the subject. In some instances, the electrical stimulation enables voluntary control of the center of mass by the subject. In certain instances, voluntary control of the center of mass includes maintaining the center of mass of the subject over a base of support. In certain embodiments, the electrical stimulation enables voluntary control by the subject sufficient to perform one or more of head control, stepping, climbing, upright sitting, shifting weight, control movement or alignment of the trunk, dynamic standing with postural or weight adjustment, transition from sitting to standing, transition from stand to walk, walk to run, increasing and decreasing speed of walking, transition from standing to sitting, crawling, proning, rolling, nodding and gesturing. In some embodiments, the neuromodulation includes applying the electrical stimulation in a manner sufficient to provide for identifying and maintaining midline orientation by the subject. In some instances, the electrical stimulation provides for identifying midline orientation with bilateral hand and arm activities. For example, the bilateral hand and arm activities may include clapping or jumping jacks.

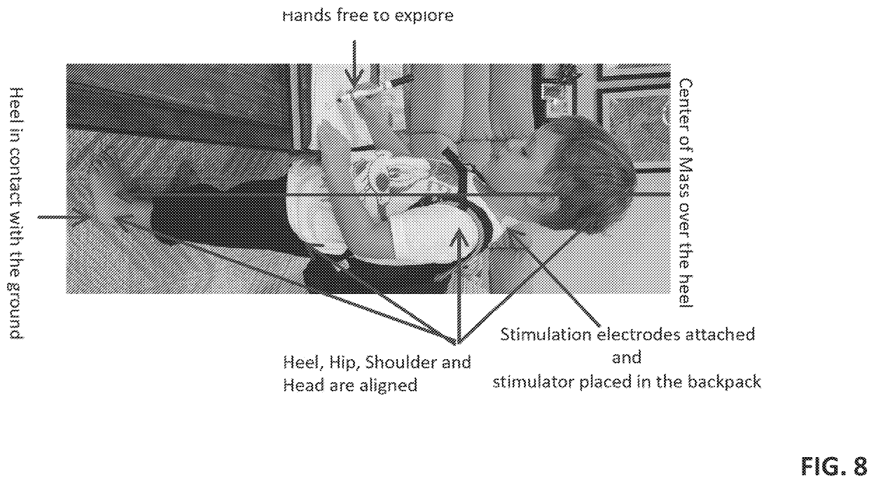

In some instances, the electrical stimulation provides for maintaining weight bearing standing by the subject. For instance, weight bearing standing with heels on the ground may be maintained by the subject. In other instances, the electrical stimulation provides for maintaining weight bearing sitting balance by the subject. For instance, weight bearing sitting balance with head over ischial tuberosities may be maintained by the subject. In certain instances, the electrical stimulation provides for maintaining a predetermined balance and posture by the subject. FIG. 8 depicts applying electrical stimulation which provides for voluntary muscle control and induced neuroplasticity (e.g., in a subject having delayed or abnormal development) according to certain embodiments. As shown in FIG. 8, electrical stimulation is non-invasively applied to the subject with electrodes which provides for the subject being able to maintain weight bearing standing with a center of mass over the heel with the hip, shoulder, head and heel being aligned while maintaining the heel in contact with the ground. During electrical stimulation, the subject's hands are free to perform tasks such as writing.

In some embodiments, the method includes maintaining the head in an upright position with the eyes parallel to the horizontal plane by the subject for appropriate visual input. In some instances, the method includes maintaining by the subject the head, trunk, pelvis and ischial tuberosities in alignment with the center of mass directly over the ischial tuberosities. In some instances, the method includes maintaining the hands and arms free to explore and interact with a surrounding space and further increase proprioceptive information from an upper extremity by the subject. In some instances, the method includes generating by the subject one or more of weight shifts, postural adjustments, external support and changes in alignment by movement of the hip and pelvis. In certain instances, the subject does not move the shoulders and ankles.

In some embodiments, neuromodulation according to methods of the present disclosure increase processing of proprioception in the brain and spinal cord. In some instances, neuromodulation as described herein increases processing of descending voluntary signals from the brain to the spinal cord of the subject. In some instances, increasing proprioception in the brain and spinal cord of the subject is sufficient to facilitate sense of touch by the subject. In some instances, increasing proprioception in the brain and spinal cord of the subject is sufficient to facilitate or improve judgement of distance by the subject. In some instances, increasing proprioception in the brain and spinal cord of the subject is sufficient to facilitate or improve judgement of object size by the subject. In some embodiments, neuromodulation increases proprioception in the brain and spinal cord of the subject sufficient to improve visual tracking by the subject. In one example, neuromodulation according to embodiments improves peripheral visual tracking by the subject. In another example, neuromodulation according to embodiments improves cross-midline visual tracking by the subject. In some embodiments, neuromodulation increases proprioception in the brain and spinal cord of the subject in a manner sufficient to change cortical visual impairment of the subject. In some instances, increasing proprioception in the brain and spinal cord of the subject is sufficient to improve visual focus of the subject.

In some embodiments, electrical stimulation according to methods of the present disclosure increase proprioception in the brain and spinal cord of the subject sufficient to facilitate or improve judgement of falling by the subject. In some instances, increasing proprioception in the brain and spinal cord of the subject is sufficient to prevent involuntary falling by the subject. In some embodiments, the electrical stimulation increases proprioception in the brain and spinal cord of the subject sufficient to provide for voluntary control of two or more of the head, hands and arm, trunk, and legs in a synchronized manner. For example, the voluntary control includes aligning two or more of the head, hands and arms, trunk, and legs. In some instances, the voluntary control includes maintaining two or more of the head, hands and arms, trunk, and legs in alignment with the center of mass directly over the base of support while walking.

In some embodiments, neuromodulation includes applying the electrical stimulation in a manner sufficient to increase self-motivation, excitement and engagement in activities by the subject. In some instances, neuromodulation increases self-initiated communication, such as non-verbal communication including but not limited to one or more of gestures, eye tracking, eye movement, head nodding, smiling, crying and laughing. In some instances, neuromodulation increases verbal communication by the subject. In some embodiments, the method includes providing one or more of verbal and tactile queues to the subject. In some instances, the verbal or tactile queues are sufficient to allow the subject to voluntarily correct an error. In certain instances, physical assistance is provided to the subject only after the subject has committed an error. For instance, assistance is not provided during or prior to the error being committed.

In some embodiments, increasing proprioception in the brain and spinal cord of the subject is sufficient to increase spatial recognition by the subject. In some instances, the spatial recognition includes informing the subject as to where one or more parts of the body are in space. In some instances, neuromodulation increases proprioception in the brain and spinal cord of the subject in when the subject is in prone position, the center of mass is in the pelvis with the ground reaction forces acting on the anterior surface of the body. In some instances, neuromodulation increases proprioception in the brain and spinal cord of the subject when the subject is in sitting position, the center of mass is directly over the ischial tuberosities. In some instances, neuromodulation increases proprioception in the brain and spinal cord of the subject when the subject is in quadruped position, the center of mass is in between the knees and hands and the ground reaction forces are at the heels of the hands, the knees and the feet. In some instances, neuromodulation increases proprioception in the brain and spinal cord of the subject when the subject is standing on a two-leg position, the center of mass is directly in between the two feet, over the heels. In some instances, neuromodulation increases proprioception in the brain and spinal cord of the subject when the subject is on a one-leg position, the center of mass is directly over the heel in contact with the ground.

In some embodiments, the electrical stimulation is applied to the spinal cord of the subject in a manner sufficient to delay or prevent detrusor overactivity in the subject. In some instances, the electrical stimulation delays or prevents detrusor overactivity in the subject during post-injury spinal shock. In some instances, the electrical stimulation reduces spasticity of the detrusor and urethral sphincter. In certain embodiments, the neuromodulation increases voluntary control of the urethral sphincter in the subject to allow contraction and relaxation of the muscle based on whether the subject intends to store urine or void urine. In some instances, the subject is capable of one or more of storing urine in the bladder and voluntarily voiding the urine from the bladder during electrical stimulation. In other instances, the subject is capable of one or more of storing urine in the bladder and voluntarily voiding the urine from the bladder in the absence of active electrical stimulation. In other instances, the subject is capable of one or more of voluntarily contracting the detrusor and simultaneously relaxing the urethral sphincter in the absence of active electrical stimulation. In certain embodiments, neuromodulation is sufficient to increase sense by the subject of bladder fullness. In other embodiments, neuromodulation is sufficient to increase bladder capacity of the subject.

In some embodiments, the electrical stimulation is applied to the spinal cord of the subject in a manner sufficient to facilitate voluntary delayed voiding contraction. In some instances, voiding contraction is delayed by an applied voluntary increase in urethral pressure by the subject. In certain instances, the voluntary increase in urethral pressure is applied in a sustained manner. In certain instances, the voluntary increase in urethral pressure is not applied in a spastic manner. In some embodiments, the electrical stimulation is applied to the spinal cord of the subject in a manner sufficient to facilitate voluntary detrusor contraction. In some embodiments, the electrical stimulation is applied to the spinal cord of the subject in a manner sufficient to facilitate a decrease in urethral pressure in response to voluntary detrusor contraction. In some instances, the frequency of voluntary voids increases in the absence of active stimulation. In some instances, the volume of voluntary voids increases in the absence of active stimulation. In some instances, the number of catheters used decreases in the absence of active stimulation.

In some embodiments, the electrical stimulation is applied to the spinal cord of the subject in a manner sufficient to increase one or more of voluntary initiation and voluntary completion of bowel movement by the subject. In some instances, neuromodulation is sufficient to increase sense by the subject of bowel fullness. In some instances, the electrical stimulation is applied to the spinal cord of the subject in a manner sufficient to facilitate voluntary contractions of one or more of the anus, rectum and other bowel sections.

In certain embodiments, the electrical stimulation is applied to the spinal cord of the subject in a manner sufficient to increase one or more voluntary sexual function by the subject. In some instances, the electrical stimulation is applied to the spinal cord of the subject in a manner sufficient to facilitate voluntary generation of psychogenic erection by the subject. In some instances, the electrical stimulation is applied to the spinal cord of the subject in a manner sufficient to facilitate voluntary generation of reflex erection by the subject. In certain instances, the electrical stimulation is applied to the spinal cord of the subject in a manner sufficient to facilitate voluntary ejaculation by the subject. In some embodiments, the electrical stimulation is applied to the spinal cord of the subject in a manner sufficient to facilitate performance of sexual intercourse by the subject. In certain instances, the electrical stimulation is applied to the spinal cord of the subject in a manner sufficient to increase or improve sense of sexual function by the subject.

In some embodiments, methods include neuromodulation in a subject for treating an accelerated aging condition caused by abnormal or delayed brain development in a subject. In some instances, methods include administering electrical neuromodulation to the spinal cord of the subject in a manner sufficient to slow down one or more aging milestones. In some instances, the term “aging milestones” is used herein in its conventional sense to refer to a condition which generally occurs at a particular age in the subject. In some instances, the subject experiences aging milestones at an age that is younger than which generally occurs in a normal subject (i.e., at an accelerated age). For example, the subject may be diagnosed as having cerebral palsy and aging milestones may occur in the subject 1 month or more before the condition generally occurs in a normal subject, such as 2 months or more, such as 3 months or more, such as 6 months or more, such as 9 months or more, such as 12 months or more, such as 2 years or more, such as 3 years or more, such as 5 years or more and including where the the aging milestone occurs 10 years or more before the condition generally occurs in a normal subject.

In some embodiments, the subject suffers from non-traumatic brain damage that occurred within about 2-3 years from birth. In some embodiments, the subject is 5 years old or younger. In some instances, methods include commencing treatment of the subject with the neuromodulation before the subject is 5 years old.

In some instances, the subject exhibits a symptom of or is diagnosed with a deterioration of walking capabilities associated with aging. In some instances, the subject exhibits a symptom of or is diagnosed with mental and physical fatigue associated with aging. In some instances, the subject exhibits a symptom of or is diagnosed with a visual deficit associated with aging. In some instances, the subject exhibits a symptom of or is diagnosed with a hearing deficit associated with aging. In some instances, the subject exhibits a symptom of or is diagnosed with cardiovascular disease or complications associated with aging, such as arterial hypertension, coronary artery disease. In some instances, the subject exhibits a symptom of or is diagnosed with one or more gastrointestinal indications (e.g., constipation or fecal incontinence) or diseases associated with aging. In some instances, the subject exhibits a symptom of or is diagnosed with speech impairment associated with aging. In some instances, the subject exhibits a symptom of or is diagnosed with impairment of chewing or swallowing associated with aging. In some instances, the subject exhibits a symptom of or is diagnosed with pain during sitting or standing that is associated with aging. In some instances, the subject exhibits a symptom of or is diagnosed with scoliosis associated with aging. In some instances, the subject exhibits a symptom of or is diagnosed with seizures associated with aging. In some instances, the subject exhibits a symptom of or is diagnosed with genitourinary dysfunction associated with aging, such as one or more of irritable bladder, bladder dysfunction, frequent urination, ureteral reflex, hypotonic enlarged bladder, frequent urinary tract infection and urinary incontinence. In some instances, the subject exhibits a symptom of or is diagnosed with sexual dysfunction associated with aging, for example where the sexual dysfunction includes one or more of sexual desire and voluntary movement during sexual activities.

In some embodiments, the subject exhibits one or more of: a) stiff muscles and exaggerated reflexes (spasticity); b) variations in muscle tone, such as being either too stiff or too floppy; c) stiff muscles with normal reflexes (rigidity); d) lack of balance and muscle coordination (ataxia); e) tremors or jerky involuntary movements; f) slow, writhing movements; g) favoring one side of the body, such as only reaching with one hand or dragging a leg while crawling; h) difficulty walking, such as walking on toes, a crouched gait, a scissors-like gait with knees crossing, a wide gait or an asymmetrical gait; and i) difficulty with fine motor skills, such as buttoning clothes or picking up utensils. In some embodiments, the subject exhibits one or more of: a) delays in speech development; b) difficulty speaking; c) difficulty with sucking, chewing or eating; and d) excessive drooling or problems with swallowing. In some embodiments, the subject exhibits one or more: a) delays in reaching motor skills milestones, such as sitting up or crawling; b) learning difficulties; c) intellectual disabilities; d) delayed growth, resulting in smaller size than would be expected. In some embodiments, the subject exhibits one or more of: a) seizures (epilepsy); b) difficulty hearing; c) problems with vision and abnormal eye movements; d) abnormal touch or pain sensations; e) bladder and bowel problems, including constipation and urinary incontinence; and f) mental health conditions, such as emotional disorders and behavioral problems. In some embodiments, the subject has, exhibits a symptom of or is diagnosed with cerebral palsy. In some instances, the aging is associated with cerebral palsy. In some instances, the subject has, exhibits a symptom of or is diagnosed with spastic cerebral palsy. In some instances, the spastic cerebral palsy is characterized by one or more of spastic diplegia, spastic hemiplegia an spastic quadriplegia. In some instances, the subject has, exhibits a symptom of or is diagnosed with dyskinetic cerebral palsy. In some instances, the dyskinetic cerebral palsy is characterized by one or more of athetoid, choreoathetoid and dystonic. In some instances, the subject has, exhibits a symptom of or is diagnosed with ataxias cerebral palsy. In some instances, the subject has, exhibits a symptom of or is diagnosed with mixed cerebral palsy.

In some embodiments, methods of the present disclosure are sufficient to move the peak performance age of the subject to an older age, such as by 1 month or more, such as by 2 months or more, such as by 3 months or more, such as by 6 months or more, such as by 9 months or more, such as by 12 months or more, such as by 2 years or more, such as by 3 years or more, such as by 5 years or more and including by 10 years or more. In some embodiments, methods include increasing peak performance by the subject, such as by 5% or more, such as by 10% or more, such as by 25% or more, such as by 50% or more, such as by 75% or more, such as by 100% or more, such as by 2-fold or more, such as by 3-fold or more, such as by 5-fold or more, such as by 10-fold or more.

In some embodiments, neuromodulation includes applying the electrical stimulation in a manner sufficient to induce neuroplasticity of the brain and spinal cord neural networks resulting in acceleration of developmental milestones initially delayed due to abnormal brain development and slowing down aging milestones initially accelerated due to abnormal brain development. In some embodiments, methods include applying the electrical stimulation at a frequency and amplitude sufficient to decrease symptoms of aging. In some embodiments, methods include applying the electrical stimulation at a frequency and amplitude sufficient to extend the life span of the subject, such as by 1 month or more, such as by 2 months or more, such as by 3 months or more, such as by 6 months or more, such as by 9 months or more, such as by 12 months or more, such as by 2 years or more, such as by 3 years or more, such as by 5 years or more and including by 10 years or more. In some embodiments, the neuromodulation is administered to the subject at predetermined intervals for 1 week or more, such as for 2 weeks or more, such as for 3 weeks or more, such as for 4 weeks or more, such as for 1 month or more, such as for 2 months or more, such as for 3 months or more, such as for 6 months or more, such as for 9 months or more, such as for 12 months or more, such as for 2 years or more, such as for 3 years or more, such as for 4 years or more, such as for 5 years or more, such as for 10 years or more and including for 25 years or more. In some embodiments, methods include applying the electrical stimulation at a frequency and amplitude sufficient to maintain functionality and extending the age at which said subject is able to perform at their peak. In some embodiments, methods include applying the electrical stimulation in a manner sufficient to enable and learn a non-patterned, non-repetitive, stochastic motor response by the subject.

In some instances, the subject the neuromodulation is administered to the subject at predetermined intervals for the entire duration of the subject's life. As described in detail above, neuromodulation may be administered to the subject at intervals of once a day or more, such as twice a day or more, such as three times a day or more and including 5 times a day or more. In some instances, the neuromodulation is administered once a week or more, such as twice a week or more, such as three times a week or more and including 5 times a week or more.

In some instances, methods include applying the electrical stimulation at a frequency and amplitude sufficient to reduce or prevent deterioration of walking capabilities associated with aging. In some instances, methods include applying the electrical stimulation at a frequency and amplitude sufficient to reduce or prevent one or more of mental and physical fatigue associated with aging. In some instances, methods include applying the electrical stimulation at a frequency and amplitude sufficient to reduce or prevent visual deficit associated with aging. In some instances, methods include applying the electrical stimulation at a frequency and amplitude sufficient to reduce or prevent hearing deficit associated with aging. In some instances, methods include applying the electrical stimulation at a frequency and amplitude sufficient to reduce or prevent cardiovascular disease or complications associated with aging. In some instances, methods include applying the electrical stimulation at a frequency and amplitude sufficient to reduce or prevent one or more gastrointestinal indications or diseases associated with aging. In some instances, methods include applying the electrical stimulation at a frequency and amplitude sufficient to reduce or prevent one or more of constipation and fecal incontinence associated with aging. In some instances, methods include applying the electrical stimulation at a frequency and amplitude sufficient to reduce or prevent speech impairment associated with aging. In some instances, methods include applying the electrical stimulation at a frequency and amplitude sufficient to improve impairment of one or more of chewing and swallowing associated with aging. In some instances, methods include applying the electrical stimulation at a frequency and amplitude sufficient to reduce or prevent pain during sitting or standing that is associated with aging. In some instances, methods include applying the electrical stimulation at a frequency and amplitude sufficient to reduce or prevent scoliosis associated with aging. In some instances, methods include applying the electrical stimulation at a frequency and amplitude sufficient to reduce or prevent the occurrence of seizures associated with aging. In some instances, methods include applying the electrical stimulation at a frequency and amplitude sufficient to reduce or prevent genitourinary dysfunction associated with aging. In certain instances, the genitourinary dysfunction includes one or more of irritable bladder, bladder dysfunction, frequent urination, ureteral reflex, hypotonic enlarged bladder, frequent urinary tract infection and urinary incontinence. In some instances, methods include applying the electrical stimulation at a frequency and amplitude sufficient to reduce or prevent sexual dysfunction associated with aging. In certain instances, the sexual dysfunction includes one or more of sexual desire and voluntary movement during sexual activities.

In some embodiments, methods include applying the electrical stimulation at a frequency and amplitude sufficient to one or more of attenuate, arrest or reverse bone loss associated with aging. In some instances, the bone loss is in the lower extremities of the subject, such as one or more of the legs, feet, toes, etc. In some instances, the bone loss is associated with or caused by paralysis. In some instances, the bone loss is associated with or caused by delayed development. In some instances, methods include applying the electrical stimulation at a frequency and amplitude sufficient to one or more of attenuate, arrest or reverse osteoporosis associated with aging. In some instances, the osteoporosis is is in the lower extremities of the subject. In some instances, the osteoporosis is associated with or caused by paralysis. In some instances, the osteoporosis is associated with or caused by delayed development. In some embodiments, methods include applying the electrical stimulation at a frequency and amplitude sufficient to heal a bone fracture. In some cases, methods include applying the electrical stimulation at a frequency and amplitude sufficient to regrow bone. In some instances, the bone is in need of regrowth due to a bone fracture. In some instances, the bone fracture is the result of an accidental fall. In some embodiments, methods include applying the electrical stimulation at a frequency and amplitude sufficient to change bone density as determined by CT scans or DEXA scans. In certain instances, the bone density is increased by 5% or more as determined by CT scans or DEXA scans, such as by 10% or more, such as by 20%, such as by 30% or more, such as by 40% or more, such as by 50% or more, such as by 60% or more, such as by 70% or more, such as by 80% or more and including by 90% or more.

In practicing the subject methods, electrodes are contacted with the subject. In some instances, the electrodes are contacted with the skin surface of the subject. As described in greater detail below, in certain embodiments the electrodes are contacted with the skin surface using a spring loaded harness which ensures sufficient contact with the subject to apply the electrical stimulation to the spinal cord of the subject. In certain embodiments the subject methods include applying transcutaneous electrical stimulation to a subject, where the method involves providing an electrical stimulator as described herein where the stimulator stores (transiently or non-transiently) one or more stimulation programs and one or more channels of the stimulator are electrically coupled to one or more transcutaneous stimulation electrodes contact with the subject (e.g., the skin surface of the subject's body) and operating the stimulator according to one or more programs to provide transcutaneous electrical stimulation to the subject. In certain embodiments the subject include applying epidural electrical stimulation to a subject, where the method involves providing an electrical stimulator where the stimulator stores (transiently or non-transiently) one or more stimulation programs and one or more channels of the stimulator are electrically coupled to one or more epidural stimulation electrodes contacted with the subject; and operating the stimulator according to one or more programs to provide epidural electrical stimulation to said subject. In certain embodiments the stimulator is configured to provide transcutaneous stimulation at one location, or at two or more locations, or at three or more locations, or at four or more locations on the subject and/or the stimulator is configured to provide epidural stimulation at one location, or at two or more locations, or at three or more locations, or at four or more locations on the subject. In certain embodiments all of the active channels of the stimulator provide transcutaneous electrical stimulation. In certain embodiments all of the active channels of the stimulator provide epidural stimulation. In certain embodiments one or more stimulator channels are configured to provide transcutaneous electrical stimulation, while other channels are configured to provide epidural electrical stimulation.

In certain embodiments the transcutaneous and/or epidural stimulation is provided by one or more of the following stimulation patterns on one or more independently controlled channels: i) one or more of a trapezoidal monophasic waveform and a trapezoidal biphasic waveform. In some instances, the ramp rate for the trapezoidal waveform varies from 0.001 ms to 0.1 ms. In some instances the duration for which the pulse is at the max varies from 0.01 ms to 0.1 ms; ii) one or more of a triangular monophasic waveform and triangular biphasic waveform. In some instances, the ramp rate for the triangular waveform varies from 0.001 ms to 0.1 ms. In some instances the duration for which the pulse is at the max varies from 0.01 ms to 0.1 ms; iii) an asymmetrical biphasic waveform. In some instances, the ramp rate for the asymmetrical biphasic waveform varies from 0.001 ms to 0.1 ms. In some instances the duration for which the pulse is at the max varies from 0.01 ms to 0.1 ms; iv) a double monophasic waveform. In some instances, the ramp rate for the double monophasic waveform varies from 0.001 ms to 0.1 ms. In some instances the duration for which the pulse is at the max varies from 0.01 ms to 0.1 ms; and a v) a monophasic waveform. In some instances, the ramp rate for the monophasic waveform varies from 0.001 ms to 0.1 ms. In some instances the duration for which the pulse is at the max varies from 0.01 ms to 0.1 ms. In certain embodiments the stimulator provides the same stimulation modality and stimulation parameters on 2 or more different channels or on 3 or more different channels, or on 4 or more different channels. In certain embodiments the stimulator provides a different stimulation modality and/or different stimulation parameters on 2 or more different channels or on 3 or more different channels, or on 4 or more different channels. In certain embodiments, pulsing is integrated into the same electrode and pulsed simultaneously. In some instances, positive and negative waveforms are pulsed simultaneously through the same electrode and stitched together to generate a unique user defined waveform.

In some embodiments, the transcutaneous and/or epidural stimulation is provided with one or more of the aforementioned waveform patterns in a manner sufficient to achieve a predetermined threshold intensity. In some instances, the predetermined threshold intensity is a preset pulse amplitude, such as a pulse amplitude of from 1 mA to 500 mA, such as from 50 mA to 200 mA, including a pulse amplitude of about 100 mA as described in greater detail below. In some instances, the predetermined threshold intensity is an intensity that is determined based on a particular goal such as where some functional goal is achieved (e.g., improve voluntary control of joints and muscles of lower extremity, upper extremity, head, neck, facial muscles, sphincters (bladder/bowel), pelvic floor, abdominal, diaphragm, throat muscles, autonomic control of organs, including bladder bowel cardiovascular sexual breathing functions).

In some instances, the transcutaneous and/or epidural stimulation is provided at a rate where the maximum intensity of each waveform is applied for a duration of from 0.001 ms to 0.1 ms, such as from 0.002 ms to 0.09 ms, such as from 0.003 ms to 0.08 ms, such as from 0.004 ms to 0.07 ms, such as from 0.005 ms to 0.06 ms, such as from 0.007 ms to 0.05 ms, such as from 0.008 ms to 0.04 ms, such as from 0.009 ms to 0.05 ms and including from 0.01 ms to 0.04 ms.

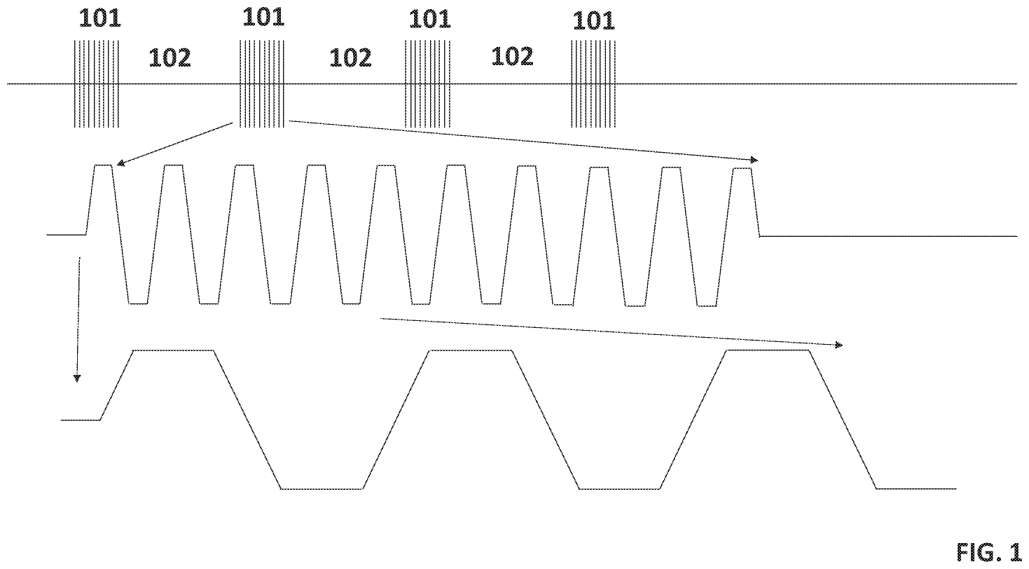

FIG. 1 depicts a trapezoidal waveform pattern for transcutaneous and/or epidural stimulation according to certain embodiments. FIG. 1 depicts high frequency components 101 and low frequency components 102 which alternate during application of the electrical stimulation. Each high frequency component includes trapezoidal waveforms. In some instances, the trapezoidal monophasic waveform is a stronger waveform as compared to the trapezoidal biphasic waveform. In some instances the ramp rate varies from 0.001 ms to 0.1 ms. In some instances the duration for which the pulse is at the max varies from 0.01 ms to 0.1 ms.

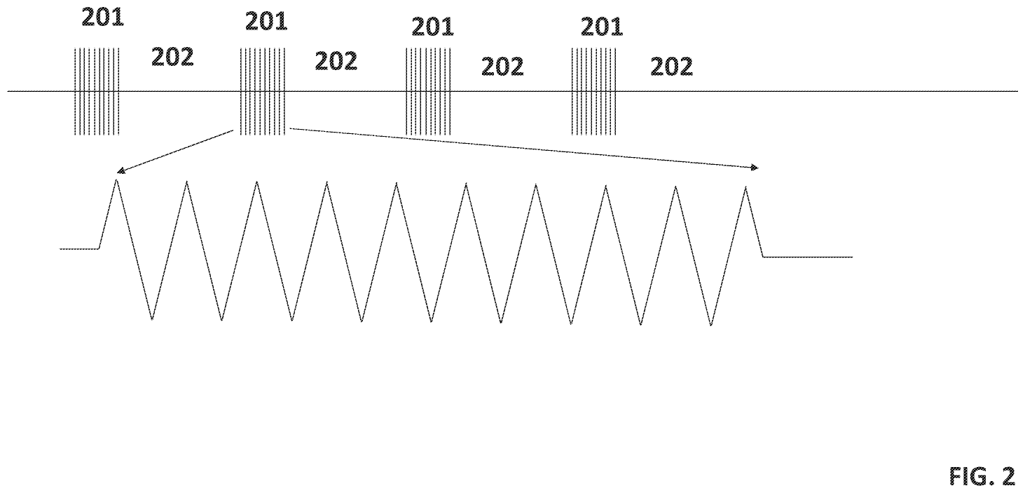

FIG. 2 depicts a triangular waveform pattern for transcutaneous and/or epidural stimulation according to certain embodiments. FIG. 2 depicts high frequency components 201 and low frequency components 202 which alternate during application of the electrical stimulation. Each high frequency component includes triangular waveforms having both a monophasic and biphasic component. In some instances the ramp rate varies from 0.001 ms to 0.1 ms. In some instances the duration for which the pulse is at the max varies from 0.01 ms to 0.1 ms.

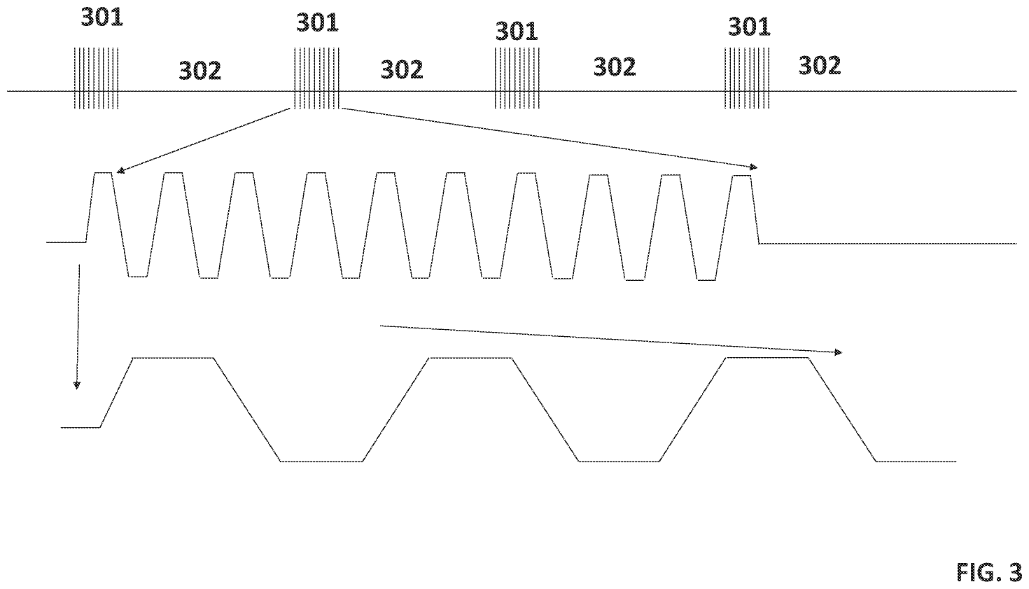

FIG. 3 depicts an asymmetrical biphasic waveform pattern for transcutaneous and/or epidural stimulation according to certain embodiments. FIG. 3 depicts high frequency components 301 and low frequency components 302 which alternate during application of the electrical stimulation. As shown in FIG. 3, the positive phase of electrical stimulation has a different amplitude as compared to negative phase of electrical stimulation. FIG. 3 depicts the trapezoidal waveform where the positive phase has a greater amplitude as compared to negative phase of each high frequency pulse. In some instances the ramp rate varies from 0.001 ms to 0.1 ms. In some instances the duration for which the pulse is at the max varies from 0.01 ms to 0.1 ms.

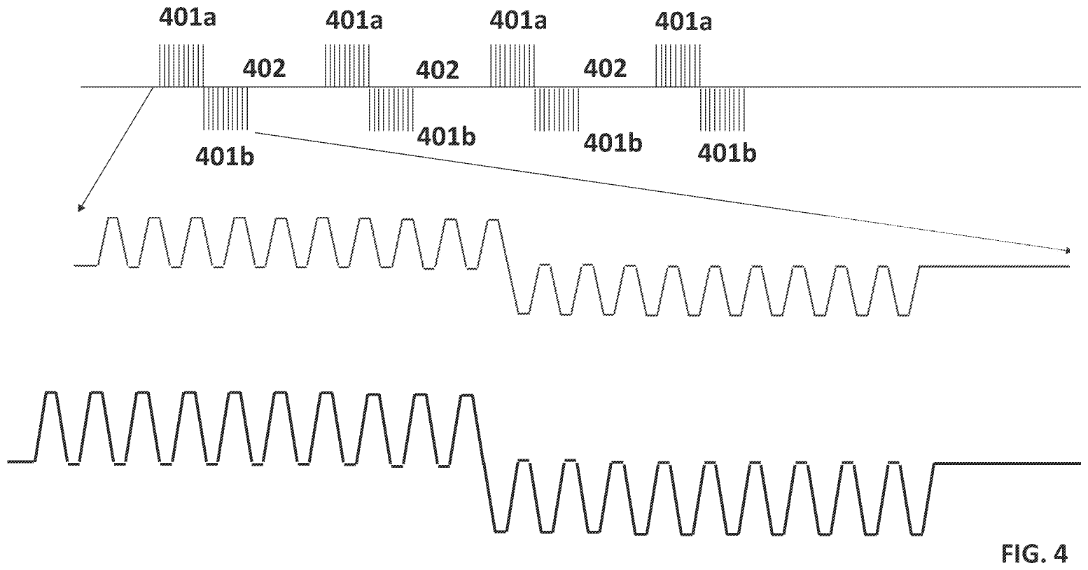

FIG. 4 depicts a double monophasic waveform pattern for transcutaneous and/or epidural stimulation according to certain embodiments. FIG. 4 depicts high frequency components 401a and 401b which have different phases which alternate with a low frequency component 402 during application of the electrical stimulation. The double monophasic waveform pattern according to certain embodiments includes a positive burst (401a) followed by a negative burst (401b). FIG. 4 depicts a trapezoidal double monophasic waveform pattern. In some instances the ramp rate varies from 0.001 ms to 0.1 ms. In some instances the duration for which the pulse is at the max varies from 0.01 ms to 0.1 ms.

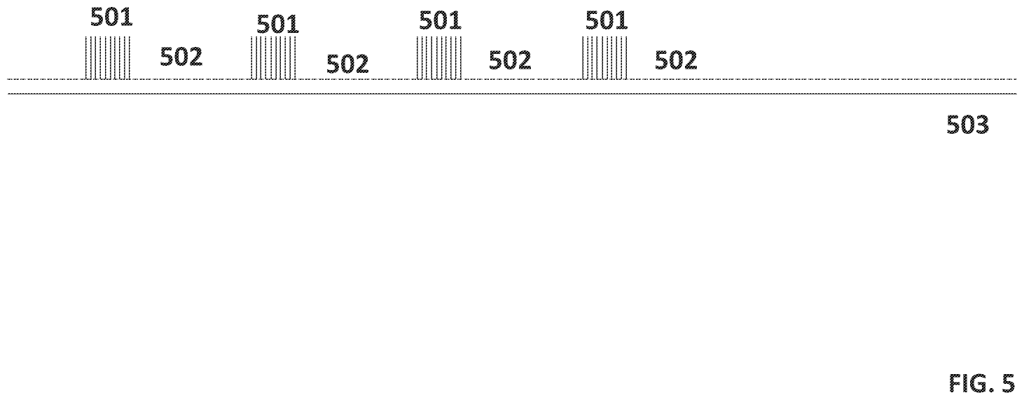

FIG. 5 depicts a monophasic waveform with opposite DC offset to balance the applied voltage from the monophasic waveform for transcutaneous and/or epidural stimulation according to certain embodiments. As depicted in FIG. 5, the applied stimulation includes a positive phase waveform that is applied having a high frequency components 501 and low frequency components 502 where the charge of the applied voltage is balanced by a continuously applied DC offset 503. In some instances the ramp rate varies from 0.001 ms to 0.1 ms. In some instances the duration for which the pulse is at the max varies from 0.01 ms to 0.1 ms.

In some embodiments, the one or more applied waveforms further includes a DC offset. In some instances, the DC offset is continuously applied in simultaneously with the applied waveforms of transcutaneous and/or epidural stimulation. In other instances, the DC offset is applied at a predetermined time after the applied waveforms of transcutaneous and/or epidural stimulation, such as 0.001 milliseconds or more after the waveforms of transcutaneous and/or epidural stimulation are applied, such as 0.005 milliseconds or more, such as 0.01 milliseconds or more, such as 0.05 milliseconds or more, such as 0.1 milliseconds or more, such as 0.5 milliseconds or more, such as 1 millisecond or more, such as 2 milliseconds or more, such as 3 milliseconds or more, such as 4 milliseconds or more, such as 5 milliseconds or more, such as 6 milliseconds or more, such as 7 milliseconds or more, such as 8 milliseconds or more, such as 9 milliseconds or more and including 10 milliseconds or more after the waveforms of transcutaneous and/or epidural stimulation are applied. In certain instances, the DC offset is applied in predetermined intervals where the DC offset is on for a predetermined period of time followed by a period of time where the DC is off. In some instances, intervals of applying the DC offset include where the DC offset is on for a duration of 0.001 milliseconds or more, such as 0.005 milliseconds or more, such as 0.01 milliseconds or more, such as 0.05 milliseconds or more, such as 0.1 milliseconds or more, such as 0.5 milliseconds or more, such as 1 millisecond or more, such as 2 milliseconds or more, such as 3 milliseconds or more, such as 4 milliseconds or more, such as 5 milliseconds or more, such as 6 milliseconds or more, such as 7 milliseconds or more, such as 8 milliseconds or more, such as 9 milliseconds or more and including 10 milliseconds or more. In some instances, intervals of applying the DC offset include where the DC offset is off for 1 millisecond or more, such as 2 milliseconds or more, such as 3 milliseconds or more, such as 4 milliseconds or more, such as 5 milliseconds or more, such as 6 milliseconds or more, such as 7 milliseconds or more, such as 8 milliseconds or more, such as 9 milliseconds or more, such as 10 milliseconds or more, such as 15 milliseconds or more, such as 20 milliseconds or more, such as 25 milliseconds or more, such as 30 milliseconds or more, such as 35 milliseconds or more, such as 40 milliseconds or more, such as 45 milliseconds or more, such as 50 milliseconds or more and including intervals where the DC offset is off for 100 milliseconds or more.

In certain embodiments, the DC offset is constantly on and a second electrode is used to remove charge from the applied electrical stimulation. In some instances, the applied DC offset has a pulse amplitude that is proportional to the amplitude of the waveform of electrical stimulation. In some instances, the pulse amplitude of the DC offset is proportional in an amount sufficient to compensate for the amplitude of the waveform of the electrical stimulation.

In some embodiments, the electrical stimulation is applied to the spinal cord of the subject at a pulse frequency of 5 Hz or more, such as at a pulse frequency of 25 Hz or more and including about a pulse frequency of about 30 Hz. In some embodiments, the electrical stimulation is applied to the spinal cord of the subject with a pulse amplitude of from 1 mA to 500 mA, such as from 50 mA to 200 mA, including a pulse amplitude of about 100 mA. In some embodiments, the electrical stimulation has a positive phase having a pulse amplitude of from 1 mA to 500 mA, such as from 50 mA to 200 mA, including a pulse amplitude of about 100 mA. In some instances, the electrical stimulation has a negative phase having a pulse amplitude of from 1 mA to 500 mA, such as from 50 mA to 200 mA, including a pulse amplitude of about 100 mA. As described above, in some instances, the electrical stimulation has an asymmetrical biphasic waveform where the positive phase has a different amplitude from the negative phase. In certain instances, where the positive phase and the negative phase have different amplitudes, the positive phase may have an amplitude that is greater than the negative phase by 1 mA or more, such as by 2 mA or more, such as by 3 mA or more, such as by 4 mA or more, such as by 5 mA or more, such as by 10 mA or more, such as by 25 mA or more, such as by 50 mA or more and including by 100 mA or more. In other instances, where the positive phase and the negative phase have different amplitudes, the negative phase may have an amplitude that is greater than the positive phase by 1 mA or more, such as by 2 mA or more, such as by 3 mA or more, such as by 4 mA or more, such as by 5 mA or more, such as by 10 mA or more, such as by 25 mA or more, such as by 50 mA or more and including by 100 mA or more. When a DC offset is applied, the DC offset amplitude may be from 0.1 mA to 10 mA, such as from 0.5 mA to 2.5 mA, including a DC offset amplitude of about 1.5 mA. The applied DC offset may be a pulsed DC offset or a continuously applied DC offset.

In some embodiments, each waveform has a high frequency component and a low frequency component. In some instances, the high frequency component has a frequency of from 1 KHz to 100 KHz, such as fro 2 KHz to 75 KHz, such as from 3 KHz to 50 KHz, such as from 4 KHz to 25 KHz, such as from 5 KHz to 15 KHz, including a high frequency component of about 10 KHz. For example, the high frequency component may have a frequency that ranges from 1 KHz to 25 KHz. In some instances, the high frequency component is the same during each interval of applied electrical stimulation. In other instances, the high frequency component is varied during each interval of applied electrical stimulation. In some instances, the low frequency component has a frequency of from 1 Hz to 500 Hz, such as from 2 Hz to 450 Hz, such as from 3 Hz to 400 Hz, such as from 4 Hz to 350 Hz, such as from 5 Hz to 300 Hz, such as from 10 Hz to 250 Hz, such as from 25 Hz to 200 Hz and including from 50 Hz to 150 Hz. For example, the low frequency component may be about 100 Hz. In some embodiments, each applied waveform has a high frequency component and a low frequency component where the high frequency component provides an analgesic effect and the low frequency component provides for the neuromodulation of the nervous system as described herein. In certain instances, the high frequency component is sufficient to provide an analgesic effect to the skin of the subject and the low frequency component is sufficient to tune spinal cord neurons to achieve the desired functional goals (i.e., the functional goals of neuromodulation)

In certain embodiments one or more channels of the electrical stimulator provide amplitude modulated dynamic stimulation. In certain embodiments one or more channels of the electrical stimulator provide provides frequency modulated dynamic stimulation. In certain embodiments the frequency modulated dynamic stimulation ranges in frequency from about 1 Hz to about 1000 Hz. In certain embodiments the dynamic stimulation is sourced from a biosignal (e.g., a signal derived from an EMG, and EEG, or an EKG). In certain embodiments the biosignal is recorded from a mammal (e.g., from a human or from a non-human primate). In certain embodiments the biosignal includes a biosignal recorded from a mammal when the mammal is standing, stepping, moving the arms, storing/emptying the bladder, storing/emptying the bowel.

In certain embodiments, at least one channel of the transcutaneous and/or epidural stimulation is applied over or more regions straddling or spanning a region selected from the group consisting of the brainstem, C0-C1, C0-C2, C0-C3, C0-C4, C0-C5, C0-C6, C0-C7, C0-T1, C1-C1, C1-C2, C₁-C₃, C₁-C₄, C1-C7, C₁-C₆, C1-C7, C1-T1, C2-C2, C2-C3, C2-C4, C2-C5, C2-C6, C2-C7, C2-T1, C3-C3, C3-C4, C₃-C₅, C₃-C₆, C3-C7, C3-T1, C4-C4, C4-C5, C4-C6, C4-C7, C4-T1, C5-C5, C5-C6, C5-C7, C5-T1, C6-C6, C6-C7, C6-T1, C7-C7, and C7-T1. In certain embodiments at least one channel of the transcutaneous and/or epidural stimulation is applied over a region comprising or consisting of C2-C3 or a region therein. In certain embodiments at least one channel of the transcutaneous and/or epidural stimulation is applied at or about C3.

In certain embodiments, at least one channel of the transcutaneous and/or epidural stimulation is applied over the thoracic spinal cord or a region thereof. In certain embodiments at least one channel of the transcutaneous and/or epidural stimulation is applied over or more regions straddling or spanning a region selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, and T12-T12.

In certain embodiments, at least one channel of the transcutaneous and/or epidural stimulation is applied over the lumbar spinal cord or a region thereof. In certain embodiments at least one channel of the transcutaneous and/or epidural stimulation is applied over or more regions straddling or spanning a region selected from the group consisting of L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6. In certain embodiments at least one channel of the transcutaneous and/or epidural stimulation is applied over the coccyx.

In certain embodiments, at least one channel of the transcutaneous and/or epidural stimulation is applied over a region between T11 and L4. In certain embodiments at least one channel of the transcutaneous and/or epidural stimulation is applied applied over or more regions selected from the group consisting of T11-T12, L1-L2, and L2-L3. In certain embodiments at least one channel of the transcutaneous and/or epidural stimulation is applied LI-L2 and/or over T11-T12.

In embodiments, the electrical stimulation may be applied to the spinal cord of the subject in intervals of 0.001 hours or more, such as 0.005 hours or more, such as 0.01 hours or more, such as 0.05 hours or more, such as 0.1 hours or more, such as 0.5 hours or more, such as 1 hour or more, such as 2 hours or more, such as 3 hours or more, such as 4 hours or more, such as 5 hours or more, such as 6 hours or more, such as 7 hours or more, such as 8 hours or more, such as 12 hours or more and including intervals of 16 hours or more. In some instances, each interval of applied electrical stimulation is a duration that ranges from 0.1 hours to 12 hours, such as from 0.5 hours to 11.5 hours, such as from 1 hour to 11 hours, such as from 2 hours to 10 hours, such as from 3 hours to 9 hours and including from 4 hours to 8 hours.

In some embodiments, the electrical stimulation is applied to the spinal cord of the subject once per day or more, such as twice per day or more, such as 3 times per day or more, such as 4 times per day or more, such as 5 times per day or more, such as 6 times per day or more and including 7 times per day or more. In these embodiments, the total duration that the electrical stimulation is applied may be 1 hour or more, such as 2 hours or more, such as 3 hours or more, such as 4 hours or more, such as 5 hours or more, such as 6 hours or more, such as 7 hours or more, such as 8 hours or more, including a total duration of 12 hours or more. In some instances, the electrical stimulation is applied on 1 day per week or more, such as on 2 days per week or more, such as on 3 days per week or more, such as on 4 days per week or more, such as on 5 days per week or more, such as on 6 days per week or more and including on every day of the week. In some instance, the electrical stimulation is applied to the subject once or twice per day in a cycle for a duration of 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days or 2 days or 1 day. In some instance, the electrical stimulation is applied to the subject once or twice per week in a cycle for a duration of one month, 2 months, 3 months, 4 months, 5 months, 6 months, 7 months, 8 months, 9 months, 10 months, 11 months or 12 months.

Each cycle of applied electrical stimulation may include application of one or more of the above described waveforms of electrical stimulation. In some instances, each cycle of applied electrical stimulation may include two or more different waveforms. In some instance, the electrical stimulation is applied to the subject in cycles that are repeated for 2, 3, 4, 5, 6, 7, 8 or more cycles, for a total period of 1 month or longer, 2 months or longer, 3 months or longer, 4 months or longer, 5 months or longer, 6 months or longer, 1 year, 2 years, 3 years or 4 years or more.

In some instances, methods further include applying magnetic stimulation to the spine of the subject. In some instances, the magnetic stimulation is applied concurrently with the electrical stimulation. In certain instances, the magnetic stimulation is constantly applied during the electrical stimulation. In other instances, the magnetic stimulation is applied in pulses during electrical stimulation. In these embodiments, each pulse interval of magnetic stimulation may overlap with the applied electrical stimulation, such as overlapping for 1 millisecond or more, such as for 10 milliseconds or more, such as for 100 milliseconds or more, such as for 1 second or more, such as for 10 seconds or more and including for 60 seconds or more. In other instances, the magnetic stimulation is applied in between pulses of electrical stimulation. In yet other instances, the magnetic stimulation is applied during the high frequency pulses of the electrical stimulation (as described above). In yet other instances, the magnetic stimulation is applied during the low frequency pulses of the electrical stimulation.

In other instances, the magnetic stimulation is applied sequentially with the electrical stimulation (i.e., after cessation of the electrical stimulation). In some instances, the magnetic stimulation is applied 1 second or more after cessation of the electrical stimulation, such as after 2 seconds or more, such as after 5 seconds or more, such as after 15 seconds or more, such as after 30 seconds or more, such as after 60 seconds or more, such as after 5 minutes or more and including 10 minutes or more after cessation of the electrical stimulation.

The magnetic stimulation may be applied to the spine of the subject using any convenient protocol, such as for example, with a electromagnetic coil device, a permanent magnet, a cylindrical magnet device (e.g., a cylindrical neodymium magnet), a circular coil magnet, a circular disc, a paddle, a butterfly coil and a figure of 8 coil. The magnetic flux density applied to the subject may vary, wherein in some instances the magnetic flux density applied to the subject is from 0.001 Tesla to 5 Tesla, such as from 0.005 Tesla to 4.5 Tesla, such as from 0.01 Tesla to 4 Tesla, such as from 0.05 Tesla to 3.5 Tesla, such as from 0.1 Tesla to 3 Tesla, such as from 0.5 Tesla to 2.5 Tesla and including from 1 Tesla to 2 Tesla. The magnetic field applied to the subject may vary, wherein in some instances the magnetic field applied to the subject is continuous, or pulsed at 0.1 Hz, 1 Hz, about 2 Hz, about 5 Hz, about 10 Hz, about 15 Hz, about 20 Hz, about 30 Hz, about 50 Hz or about 100 Hz. The magnetic field applied to the subject may vary, wherein in some instances the magnetic field burst applied to the subject is once every second, once every 5 secs, two times a second, five times a second, ten times a second, twenty times a second, thirty times a second or fifty times a second.

In some embodiments, the magnetic stimulation may be applied to the spinal cord of the subject in intervals of 0.001 hours or more, such as 0.005 hours or more, such as 0.01 hours or more, such as 0.05 hours or more, such as 0.1 hours or more, such as 0.5 hours or more, such as 1 hour or more, such as 2 hours or more, such as 3 hours or more, such as 4 hours or more, such as 5 hours or more, such as 6 hours or more, such as 7 hours or more, such as 8 hours or more, such as 12 hours or more and including intervals of 16 hours or more. In some instances, each interval of applied magnetic stimulation is a duration that ranges from 0.1 hours to 12 hours, such as from 0.5 hours to 11.5 hours, such as from 1 hour to 11 hours, such as from 2 hours to 10 hours, such as from 3 hours to 9 hours and including from 4 hours to 8 hours. In some instances, the duration of applied magnetic stimulation is the same as the duration of applied electrical stimulation. In some instances, the magnetic stimulation is applied at varying times during the applied electrical stimulation.

In some instances, methods further include applying mechanical stimulation to the spine of the subject. In some instances, the mechanical stimulation is applied concurrently with the electrical stimulation. In certain instances, the mechanical stimulation is a constant push of the electrode (e.g., the electrode of the electrical stimulator) into the spine of the subject during electrical stimulation. In other instances, the mechanical stimulation is applied in pulses during electrical stimulation. In these embodiments, each pulse interval of mechanical stimulation may overlap with the applied electrical stimulation, such as overlapping for 1 millisecond or more, such as for 10 milliseconds or more, such as for 100 milliseconds or more, such as for 1 second or more, such as for 10 seconds or more and including for 60 seconds or more. In other instances, the mechanical stimulation is applied in between pulses of electrical stimulation. In yet other instances, the mechanical stimulation is applied during the high frequency pulses of the electrical stimulation (as described above). In yet other instances, the mechanical stimulation is applied during the low frequency pulses of the electrical stimulation.

In some embodiments, the mechanical stimulation may be applied to the spinal cord of the subject in intervals of 0.001 hours or more, such as 0.005 hours or more, such as 0.01 hours or more, such as 0.05 hours or more, such as 0.1 hours or more, such as 0.5 hours or more, such as 1 hour or more, such as 2 hours or more, such as 3 hours or more, such as 4 hours or more, such as 5 hours or more, such as 6 hours or more, such as 7 hours or more, such as 8 hours or more, such as 12 hours or more and including intervals of 16 hours or more. In some instances, each interval of applied mechanical stimulation is a duration that ranges from 0.1 hours to 12 hours, such as from 0.5 hours to 11.5 hours, such as from 1 hour to 11 hours, such as from 2 hours to 10 hours, such as from 3 hours to 9 hours and including from 4 hours to 8 hours. In some instances, the duration of applied mechanical stimulation is the same as the duration of applied electrical stimulation. In some instances, the mechanical stimulation is applied at varying times during the applied electrical stimulation.

In some instances, methods include applying magnetic and mechanical stimulation simultaneously with the electrical stimulation. In certain instances, the magnetic and mechanical stimulation may be applied to the spinal cord of the subject in intervals of 0.001 hours or more, such as 0.005 hours or more, such as 0.01 hours or more, such as 0.05 hours or more, such as 0.1 hours or more, such as 0.5 hours or more, such as 1 hour or more, such as 2 hours or more, such as 3 hours or more, such as 4 hours or more, such as 5 hours or more, such as 6 hours or more, such as 7 hours or more, such as 8 hours or more, such as 12 hours or more and including intervals of 16 hours or more. In some instances, each interval of applied magnetic and mechanical stimulation is a duration that ranges from 0.1 hours to 12 hours, such as from 0.5 hours to 11.5 hours, such as from 1 hour to 11 hours, such as from 2 hours to 10 hours, such as from 3 hours to 9 hours and including from 4 hours to 8 hours. In some instances, the duration of applied magnetic and mechanical stimulation is the same as the duration of applied electrical stimulation. In some instances, the magnetic and mechanical stimulation is applied at varying times during the applied electrical stimulation.

According to embodiments, the subject methods enhance voluntary control of physical activity by the subject. In some embodiments, the electrical stimulation is applied to the spinal cord of the subject in a manner sufficient to maintain voluntary control of one or more of physical motor function, sensory function, vestibular function, cognitive function, autonomic function and sleep activity. In some embodiments, the electrical stimulation is applied to the spinal cord of the subject in a manner sufficient to maintain voluntary control of one or more of anxiety, depression and mood. In some instances, voluntary control of physical activity is maintained by the subject after cessation of the electrical stimulation. In some instances, the electrical stimulation is applied to the spinal cord of the subject to improve voluntary control of joints and muscles of lower extremity, upper extremity, head, neck, facial muscles, sphincters (bladder/bowel), pelvic floor, abdominal, diaphragm, throat muscles, autonomic control of organs, including bladder bowel cardiovascular sexual breathing functions. In some instances, the electrical stimulation is applied to the spinal cord of the subject to improve sensation of joints and muscles of lower extremity, upper extremity, head, neck, facial muscles, sphincters (bladder/bowel), pelvic floor, abdominal, diaphragm, throat muscles, autonomic control of organs including bladder bowel cardiovascular sexual breathing functions.

In some embodiments, the subject methods include applying the electrical stimulation to the spinal cord of the subject to improve vision in the subject, such as one or more of near sightedness, far sightedness, peripheral vision and visual acuity. In some instances, the applied electrical stimulation improves the sense of smell in the subject. In other instances, the applied electrical stimulation improves the sense of hearing by the subject. In certain instances, the applied electrical stimulation improves voice modulation by the subject, such as improving one or more of the ability to vocalize, articulation, speaking softly, speaking loudly, and duration of voice modulation by the subject.

In some instances, methods include applying electrical stimulation to the spinal cord of the subject to improve the ability to control one or more of swallowing, biting, sipping, movement of the lower jaw, movement of the tongue by the subject. In some instances, the applied electrical stimulation improves the control of facial muscles by the subject, such as improving smiling by the subject. In other instances, applying electrical stimulation improves the sense of taste, movement of the eyeballs or movement of the head and neck by the subject.

In certain embodiments, applying electrical stimulation to the spinal cord of the subject is sufficient to improve sleep by the subject, such as the ability to fall asleep faster, ability to sleep longer without waking up at night and ability to go back to sleep after waking up. In some instances, the applied electrical stimulation reduces or normalizes seizure activity by the subject. In other instances, the applied electrical stimulation reduces or normalizes the resting state of the nervous system of the subject.

In some embodiments, methods include applying the electrical stimulation to the spinal cord of the subject to treat anxiety or depression in the subject. In other embodiments, methods include applying the electrical stimulation to the spinal cord of the subject to improve vestibular function in the subject such as improving vertigo, dizziness, visual disturbance, and imbalance. In some embodiments, the applied electrical stimulation improves bladder function in the subject such as by increasing bladder capacity, increasing sensation of bladder fullness, reducing urinary incontinence, increasing voluntary control to hold, improving ability to void voluntarily, reducing the use of catheters to empty bladder. In other embodiments, applying electrical stimulation to the spinal cord of the subject improves bowel function in the subject such as by increasing sensation of bowel fullness, reducing fecal incontinence, increasing voluntary control to hold and improving the ability to defecate voluntarily. In other embodiments, applying electrical stimulation to the spinal cord of the subject improves sexual function in the subject such as by improving sensation of urogenital organs, returning the ability to have an erection, increasing lubrication, increasing sensation during erection and penetration, increasing ability for voluntary penetration, increasing ability to sustain erection for longer periods of time and increasing degree of orgasm at climax. In certain instances, the applied electrical stimulation increases sperm count, sperm mortality and vitality by the subject.

### Systems for Neuromodulating the Central Nervous System of a Subject

Aspects of the present disclosure include systems for practicing the subject methods described above for neuromodulating the central nervous system of a subject. Systems according to certain embodiments include an electrical stimulator that is configured to apply electrical stimulation to the spinal cord of a subject in a manner sufficient to maintain voluntary control of physical activity during the electrical stimulation. Systems according some instances, include an electrical stimulator that is configured to apply electrical stimulation to the spinal cord of a subject in a manner sufficient to induce neuroplasticity of the brain and spinal cord neural network of the subject. In some embodiments, systems include a wearable electrical stimulator device. In some instances, the wearable device includes a single use battery. In other instances, the wearable device includes a rechargeable battery. In certain instances, the wearable device is disposable. In some embodiments, the wearable device is configured to route wires under the clothing of the subject. In some instances, the electrical stimulator is integrated into clothing or furniture (e.g., chair or bed) or some other device which positions the electrical stimulator at a location along the spinal cord of the subject. In some embodiments, systems include one or more of a powered exoskeleton device, a powered or active orthosis, a passive orthosis, a wearable orthosis, a soft exoskeleton device, a hip orthosis, a knee orthosis, a head orthosis, an ankle orthosis, a body weight support device, a stand frame, a wheelchair, a set of crutches and a walker.

In certain embodiments, the electrical stimulator includes a set of spring-loaded electrodes that are configured to ensure hydrogel contact between the electrodes with the skin of the subject. For example, the electrical stimulator may be integrated into a belt or harness with worn springs. In some instances, the springs of the electrical stimulator device is configured to provide mechanical and vibrotactile stimulation.

In some embodiments, systems are configured to apply electrical stimulation is sufficient to integrate and reconnect the brain to the spinal cord. In some instances, systems apply electrical stimulation to the spinal cord of the subject that is sufficient to increase ascending neural signals to the brain of the subject, such as increasing cortical and voluntary sensation. In some instances, systems apply electrical stimulation to the spinal cord of the subject that is sufficient to excite neurons in the brain. In other instances, systems apply electrical stimulation to the spinal cord of the subject that is sufficient to inhibit neurons in the brain. In certain instances, systems apply electrical stimulation to the spinal cord of the subject that simultaneously excites some neurons in the brain and inhibits some neurons in the brain. In some instances, systems are configured to apply electrical stimulation to the spinal cord of the subject at a frequency and amplitude sufficient to activate sensory neurons of the spinal cord neural network. In some instances, systems are configured to apply electrical stimulation to the spinal cord of the subject at a frequency and amplitude sufficient to activate interneurons of the spinal cord neural network. In certain embodiments, systems are configured to apply electrical stimulation which does not directly activate the motor neurons of the spinal cord neural network. In some instances, systems are configured to apply electrical stimulation to the spinal cord of the subject at a frequency and amplitude which activates interneurons of the spinal cord sufficient to facilitate signal conduction to motor neurons. In some embodiments, the signal conduction to the motor neurons provides for voluntary muscle control by the subject. The voluntary muscle control may be one or more of activating one or more muscle groups, inhibiting activity by one or more muscle groups and having no impact on one or more muscle groups.

In some instances, systems apply electrical stimulation to the spinal cord of the subject that reconnects the spinal neural network with the brain of the subject. In certain instances, systems apply electrical stimulation to the spinal cord of the subject that retrains the spinal neural network of the central nervous system of the subject. In some embodiments, systems apply electrical stimulation to the spinal cord of the subject in a manner sufficient to enable a stochastic motor response by the subject. In some embodiments, the electrical stimulator component of the subject systems include one or more channels where each channel provides a transcutaneous electrical stimulation signal or an epidural stimulation signal. In certain embodiments the electrical stimulator provides one or more independently controlled stimulation channel, such as 2 or more independently controllable (programmable) stimulation channels, such as 3 or more independently controllable (programmable) stimulation channels, such as 4 or more independently controllable (programmable) stimulation channels, such as 6 or more independently controllable (programmable) stimulation channels, such as 8 or more independently controllable (programmable) stimulation channels, such as 12 or more independently controllable (programmable) stimulation channels, such as 16 or more independently controllable (programmable) stimulation channels, such as 20 or more independently controllable (programmable) stimulation channels, such as 24 or more independently controllable (programmable) stimulation channels. In certain embodiments where there is more than one channel, multiple channels may provide stimulation signals with respect to a common (e.g., neutral or ground) lead. In certain embodiments where there is more than one channel, two or more different channels may provide stimulation signals with respect to a different leads. In certain embodiments, pulsing is integrated into the same electrode and pulsed simultaneously. In some instances, positive and negative waveforms are pulsed simultaneously through the same electrode and stitched together to generate a unique user defined waveform.

Where the electrical stimulator includes more than one channel, one or more of the channels may be configured to provide a transcutaneous electrical stimulation. In other instances, where the electrical stimulator includes more than one channel, one or more of the channels may be configured to provide an epidural stimulation signal. In yet other instances, where the electrical stimulator includes more than one channel, or one or more channels may be configured to provide a transcutaneous electrical stimulation signal, while one or more other channels are configured to provide an epidural stimulation signal.

As described above, in some instances, the electrical stimulator is configured to provide one or more of the following stimulation patterns on one or more independently controlled channels: i) one or more of a trapezoidal monophasic waveform and a trapezoidal biphasic waveform. In some instances, the ramp rate for the trapezoidal waveform varies from 0.001 ms to 0.1 ms. In some instances the duration for which the pulse is at the max varies from 0.01 ms to 0.1 ms; ii) one or more of a triangular monophasic waveform and triangular biphasic waveform. In some instances, the ramp rate for the triangular waveform varies from 0.001 ms to 0.1 ms. In some instances the duration for which the pulse is at the max varies from 0.01 ms to 0.1 ms; iii) an asymmetrical biphasic waveform. In some instances, the ramp rate for the asymmetrical biphasic waveform varies from 0.001 ms to 0.1 ms. In some instances the duration for which the pulse is at the max varies from 0.01 ms to 0.1 ms; iv) a double monophasic waveform. In some instances, the ramp rate for the double monophasic waveform varies from 0.001 ms to 0.1 ms. In some instances the duration for which the pulse is at the max varies from 0.01 ms to 0.1 ms; and a v) a monophasic waveform. In some instances, the ramp rate for the monophasic waveform varies from 0.001 ms to 0.1 ms. In some instances the duration for which the pulse is at the max varies from 0.01 ms to 0.1 ms. In certain embodiments the stimulator provides the same stimulation modality and stimulation parameters on 2 or more different channels or on 3 or more different channels, or on 4 or more different channels. In certain embodiments the stimulator provides a different stimulation modality and/or different stimulation parameters on 2 or more different channels or on 3 or more different channels, or on 4 or more different channels.

In some embodiments, the electrical stimulator is configured to provide transcutaneous and/or epidural stimulation with one or more of the aforementioned waveform patterns in a manner sufficient to achieve a predetermined threshold intensity. In some instances, the predetermined threshold intensity is a preset pulse amplitude, such as a pulse amplitude of from 1 mA to 500 mA, such as from 50 mA to 200 mA, including a pulse amplitude of about 100 mA as described in greater detail below. In some instances, the predetermined threshold intensity is an intensity that is determined based on a particular goal such as where some functional goal is achieved (e.g., improve voluntary control of joints and muscles of lower extremity, upper extremity, head, neck, facial muscles, sphincters (bladder/bowel), pelvic floor, abdominal, diaphragm, throat muscles, autonomic control of organs, including bladder bowel cardiovascular sexual breathing functions).

In some embodiments, systems include an electrical stimulator that is configured to apply one or more waveforms that further include a DC offset. In some instances, the electrical stimulator is configured to continuously apply the DC offset simultaneously with the applied waveforms of transcutaneous and/or epidural stimulation. In other instances, the electrical stimulator is configured to apply the DC offset at a predetermined time after the applied waveforms of transcutaneous and/or epidural stimulation, such as 0.001 milliseconds or more after the waveforms of transcutaneous and/or epidural stimulation are applied, such as 0.005 milliseconds or more, such as 0.01 milliseconds or more, such as 0.05 milliseconds or more, such as 0.1 milliseconds or more, such as 0.5 milliseconds or more, such as 1 millisecond or more, such as 2 milliseconds or more, such as 3 milliseconds or more, such as 4 milliseconds or more, such as 5 milliseconds or more, such as 6 milliseconds or more, such as 7 milliseconds or more, such as 8 milliseconds or more, such as 9 milliseconds or more and including 10 milliseconds or more after the waveforms of transcutaneous and/or epidural stimulation are applied. In certain instances, the electrical stimulator is configured to apply the DC offset in predetermined intervals where the DC offset is on for a predetermined period of time followed by a period of time where the DC is off. In some instances, the electrical stimulator is configured to apply intervals of the DC offset where the DC offset is on for a duration of 0.001 milliseconds or more, such as 0.005 milliseconds or more, such as 0.01 milliseconds or more, such as 0.05 milliseconds or more, such as 0.1 milliseconds or more, such as 0.5 milliseconds or more, such as 1 millisecond or more, such as 2 milliseconds or more, such as 3 milliseconds or more, such as 4 milliseconds or more, such as 5 milliseconds or more, such as 6 milliseconds or more, such as 7 milliseconds or more, such as 8 milliseconds or more, such as 9 milliseconds or more and including 10 milliseconds or more. In some instances, the electrical stimulator is configured to apply intervals of the DC offset where the DC offset is off for 1 millisecond or more, such as 2 milliseconds or more, such as 3 milliseconds or more, such as 4 milliseconds or more, such as 5 milliseconds or more, such as 6 milliseconds or more, such as 7 milliseconds or more, such as 8 milliseconds or more, such as 9 milliseconds or more, such as 10 milliseconds or more, such as 15 milliseconds or more, such as 20 milliseconds or more, such as 25 milliseconds or more, such as 30 milliseconds or more, such as 35 milliseconds or more, such as 40 milliseconds or more, such as 45 milliseconds or more, such as 50 milliseconds or more and including intervals where the DC offset is off for 100 milliseconds or more.

In certain embodiments, the electrical stimulator is configured to have the DC offset constantly on and a second electrode to remove charge from the applied electrical stimulation. In some instances, the electrical stimulator is configured to apply a DC offset that has a pulse amplitude that is proportional to the amplitude of the waveform of electrical stimulation. In some instances, the pulse amplitude of the DC offset is proportional in an amount sufficient to compensate for the amplitude of the waveform of the electrical stimulation.

In some embodiments, the electrical stimulator applies stimulation to the spinal cord of the subject at a pulse frequency of 5 Hz or more, such as at a pulse frequency of 25 Hz or more and including about a pulse frequency of about 30 Hz. In some embodiments, the electrical stimulator in configured to apply electrical stimulation to the spinal cord of the subject with a pulse amplitude of from 1 mA to 500 mA, such as from 50 mA to 200 mA, including a pulse amplitude of about 100 mA. In some embodiments, the electrical stimulator in configured to apply electrical stimulation that has a positive phase having a pulse amplitude of from 1 mA to 500 mA, such as from 50 mA to 200 mA, including a pulse amplitude of about 100 mA. In some instances, the electrical stimulator in configured to apply electrical stimulation that has a negative phase having a pulse amplitude of from 1 mA to 500 mA, such as from 50 mA to 200 mA, including a pulse amplitude of about 100 mA. As described above, in some instances, the electrical stimulation has an asymmetrical biphasic waveform where the positive phase has a different amplitude from the negative phase. In certain instances, where the positive phase and the negative phase have different amplitudes, the electrical stimulator in configured to apply electrical stimulation where the positive phase has an amplitude that is greater than the negative phase by 1 mA or more, such as by 2 mA or more, such as by 3 mA or more, such as by 4 mA or more, such as by 5 mA or more, such as by 10 mA or more, such as by 25 mA or more, such as by 50 mA or more and including by 100 mA or more. In other instances, where the positive phase and the negative phase have different amplitudes, the electrical stimulator in configured to apply electrical stimulation where the negative phase has an amplitude that is greater than the positive phase by 1 mA or more, such as by 2 mA or more, such as by 3 mA or more, such as by 4 mA or more, such as by 5 mA or more, such as by 10 mA or more, such as by 25 mA or more, such as by 50 mA or more and including by 100 mA or more. When a DC offset is applied, the DC offset amplitude may be from 0.1 mA to 10 mA, such as from 0.5 mA to 2.5 mA, including a DC offset amplitude of about 1.5 mA. The applied DC offset may be a pulsed DC offset or a continuously applied DC offset.

In some embodiments, the electrical stimulator in configured to apply electrical stimulation where each waveform has a high frequency component and a low frequency component. In some instances, the high frequency component has a frequency of from 1 KHz to 100 KHz, such as from 2 KHz to 75 KHz, such as from 3 KHz to 50 KHz, such as from 4 KHz to 25 KHz, such as from 5 KHz to 15 KHz, including a high frequency component of about 10 KHz. For example, the high frequency component may have a frequency that ranges from 1 KHz to 25 KHz. In some instances, the high frequency component is the same during each interval of applied electrical stimulation. In other instances, the electrical stimulator is configured to apply a high frequency component that is varied during each interval of applied electrical stimulation. In some instances, the low frequency component has a frequency of from 1 Hz to 500 Hz, such as from 2 Hz to 450 Hz, such as from 3 Hz to 400 Hz, such as from 4 Hz to 350 Hz, such as from 5 Hz to 300 Hz, such as from 10 Hz to 250 Hz, such as from 25 Hz to 200 Hz and including from 50 Hz to 150 Hz. For example, the low frequency component may be about 100 Hz. In some embodiments, the electrical stimulator is configured to provide a stimulation pulse (burst) width on one or more channels ranging from about 0.1 ms up to about 20 ms, or up to about 10 ms, or up to about 5 ms, or up to about 4 ms, or from about 0.2 ms up to about 3 ms. In certain embodiments the stimulator provides a pulse width fixed at 1 ms at stimulation frequencies over 10 KHz.

In certain embodiments, systems include a transcutaneous or epidural stimulator for applying electrical stimulation to the spinal cord having electrical stimulator components such as transcutaneous and epidural stimulation electrodes, electrical stimulator controller (e.g., microprocessors/microcontrollers), pulse generators, pulse modulating gating units, shift generators, charge balancers, DC current controllers, monitors and biometric input controllers.

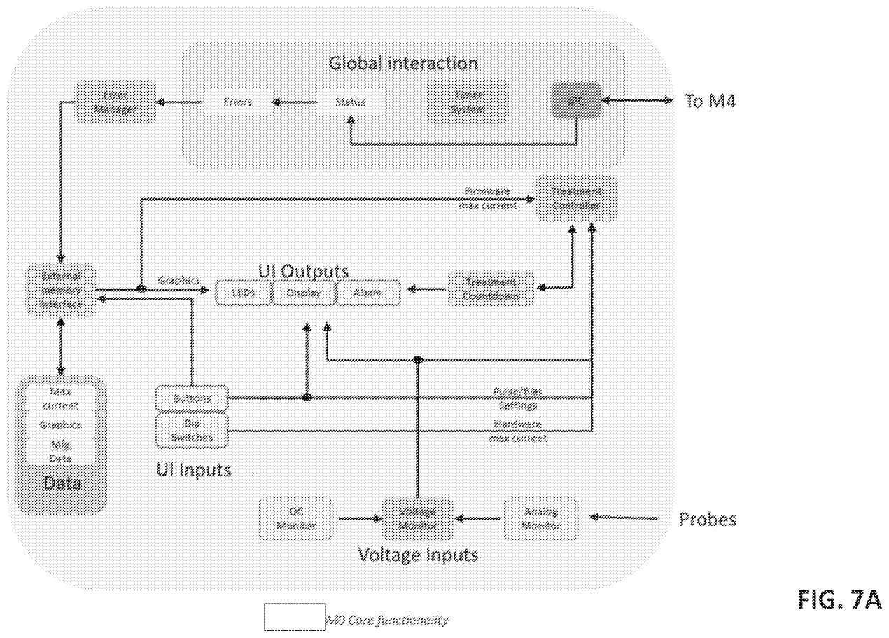

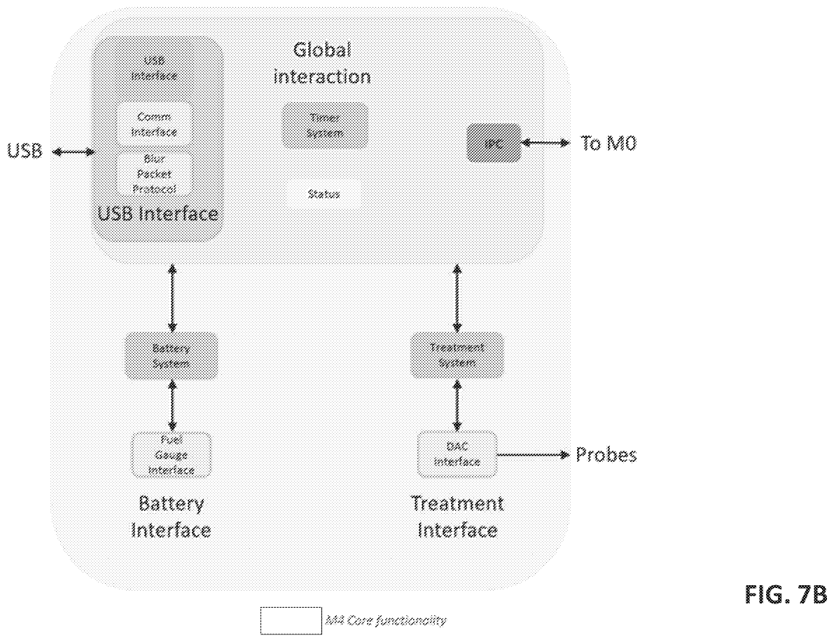

In some instances, the subject systems are modular systems that include one or more subsystems. An example of a system showing the different subsystems for applying transcutaneous or epidural stimulation according to the methods described herein is depicted in FIGS. 7A and 7B. In certain instances, the waveform generator system includes an analog voltage measurement subsystem to measure the voltage and current levels at the output channels. In some instances, the waveform generator includes a feedback system to control the output controls all bursts, current and bias settings per user settings.

In some instances, the subject systems include a display system. In certain instances, the display system includes a data storage subsystem to store the graphic data in a non-volatile fashion and a graphical display subsystem to output the graphics to a custom graphics display/OLED display module.

In some instances, the subject systems include a power supply. In some embodiments, the power supply includes a battery system. In certain embodiments, the battery system includes a battery charging subsystem which charges the battery and a fuel gauge subsystem which monitors the status of the battery.

In some instances, the subject systems include a safety monitor system. In certain instances, the safety monitor system includes an analog subsystem which monitors voltage values and a digital subsystem which monitors digital inputs. These subsystem inputs are then evaluated for validity and the system responds per SRS.

In certain embodiments, systems of interest include electrodes for contacting with the skin surface of the subject that are spring-loaded, such as where the electrode includes a spring to ensure sufficient contact between the electrode and the skin surface of the subject. In some instances, the spring loaded electrodes are sufficient to apply mechanical pressure to maintain contact between the electrodes and the spine of the subject. In some instances, the spring loaded electrodes are integrated in a harness device or a belt which can be worn by the subject. In other instances, the spring-loaded electrodes are integrated into the clothing worn by the subject. In other instances, the spring-loaded electrodes are integrated into a device which maintains contact with spine of the subject, such as for example a chair or a bed.

In some embodiments, the electrical stimulator is configured to deliver the electrical stimulation while the subject is seated. In some instances, the subject is in a supine position while the electrical stimulation is applied to the subject. In other instances, the subject is standing with weight bearing while the electrical stimulation is applied to the subject. In other instances, the subject is walking, such as on a treadmill or walking overground while the electrical stimulation is applied to the subject.

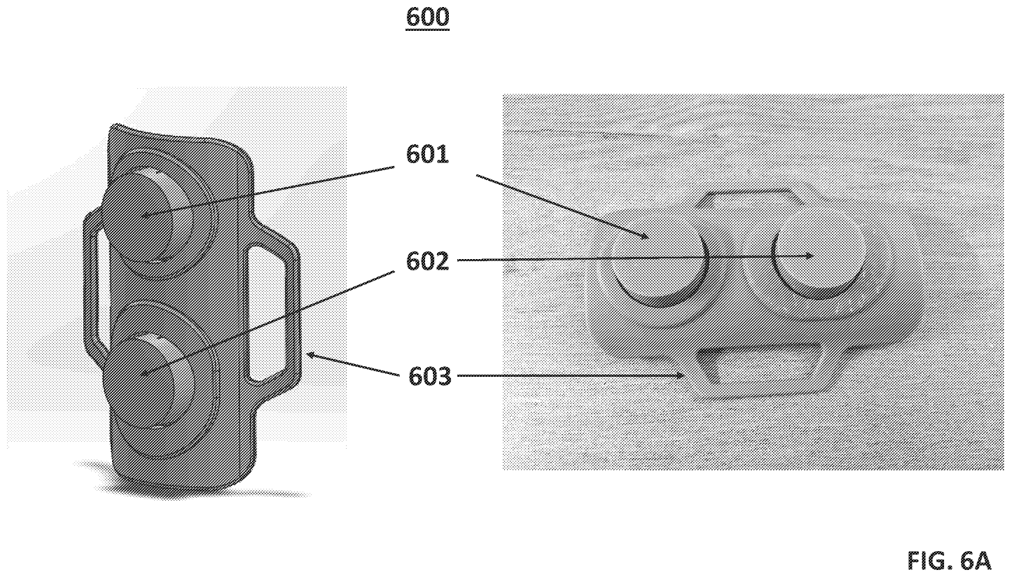

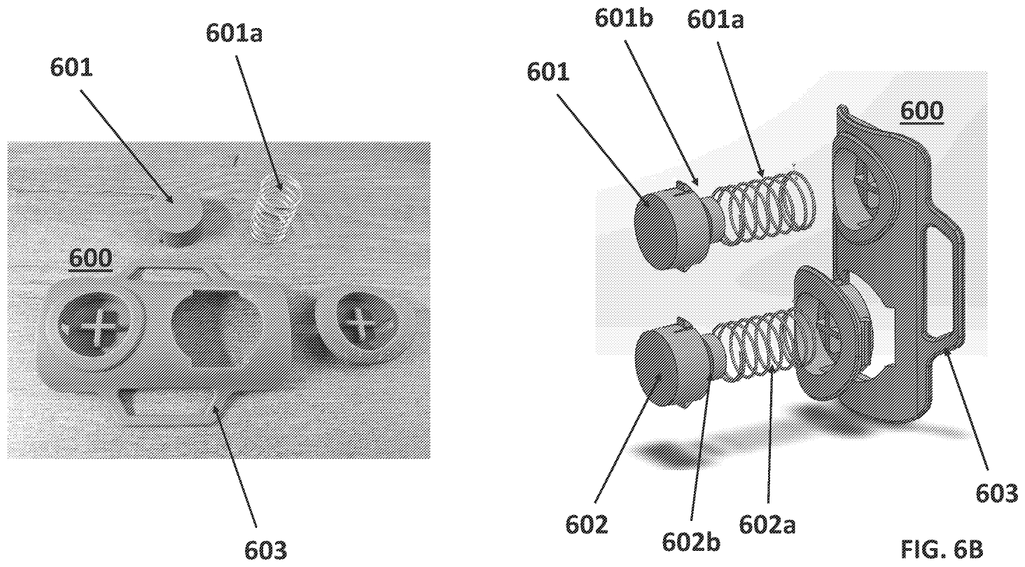

FIG. 6 depicts an electrode holder according to certain embodiments. FIG. 6A includes electrode holder 600 having discs 601 and 602 for attaching electrodes of an electrical stimulator as described herein. Electrode holder also includes loop 603 which can be used to connect to a harness or belt to worn by the subject. Discs 601 and 602 can include springs 601a and 602a as depicted in FIG. 6B. Springs 601a and 602a apply pressure to the electrode such that the electrode maintains sufficient contact with the skin of the subject during electrical stimulation. Discs 601 and 602 can also include linear actuators 601b and 602b which can be used to provide mechanical vibration and linear movement of the electrode. In some instances, linear actuators 601b and 602b provide mechanical stimulation to the spine of the subject.

Aspects, including embodiments, of the subject matter described herein may be beneficial alone or in combination, with one or more other aspects or embodiments. Without limiting the description, certain non-limiting aspects of the disclosure numbered 1-506 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of neuromodulating the central nervous system of a subject, the method comprising applying electrical stimulation to the spinal cord of a subject in a manner sufficient to maintain voluntary control of physical activity during the electrical stimulation.
2. The method according to 1, wherein applying the electrical stimulation is sufficient to integrate and reconnect the brain to the spinal cord.
3. The method according to 1, wherein the electrical stimulation is applied to the spinal cord of the subject in a manner sufficient to maintain voluntary control of one or more of physical motor function, sensory function, vestibular function, cognitive function, autonomic function and sleep activity.
4. The method according to 1, wherein the electrical stimulation is applied to the spinal cord of the subject in a manner sufficient to maintain voluntary control of one or more of anxiety, depression and mood.
5. The method according to any one of 1-4, wherein the voluntary control of physical activity is maintained by the subject after cessation of the electrical stimulation.
6. The method according to any one of 1-5, wherein applying electrical stimulation to the spinal cord of the subject is sufficient to excite neurons in the brain.
7. The method according to any one of 1-6, wherein applying electrical stimulation to the spinal cord of the subject is sufficient to inhibit neurons in the brain.
8. The method according to any one of 1-7, wherein the electrical stimulation is applied to the spinal cord of the subject in a manner sufficient to improve voluntary control of joints and muscles of lower extremity, upper extremity, head, neck, facial muscles, sphincters (bladder/bowel), pelvic floor, abdominal, diaphragm, throat muscles, autonomic control of organs, including bladder bowel cardiovascular sexual breathing functions.
9. The method according to any one of 1-7, wherein the electrical stimulation is applied to the spinal cord of the subject in a manner sufficient to improve sensation of joints and muscles of lower extremity, upper extremity, head, neck, facial muscles, sphincters (bladder/bowel), pelvic floor, abdominal, diaphragm, throat muscles, autonomic control of organs including bladder bowel cardiovascular sexual breathing functions.
10. The method according to any one of 1-9, wherein the electrical stimulation is applied to the spinal cord of the subject in a manner sufficient to retrain the spinal neural network of the central nervous system of the subject.
11. The method according to any one of 1-10, wherein the electrical stimulation is applied to the spinal cord of the subject in a manner sufficient to reconnect the spinal neural network with the brain of the subject.
12. The method according to any one of 1-11, wherein the electrical stimulation applied to spinal cord of the subject is sufficient to enhance voluntary control of physical activity by the subject.
13. The method according to any one of 1-12, wherein the electrical stimulation applied to the spinal cord of the subject is sufficient to increase ascending neural signals to the brain of the subject.
14. The method according to 13, wherein increasing ascending neural signals comprises increasing cortical and voluntary sensation.
15. The method according to any one of 1-14, wherein the electrical stimulation comprises applying at least one waveform selected from the group consisting of:
   one or more of a trapezoidal monophasic waveform and a trapezoidal biphasic waveform;
   one or more of a triangular monophasic waveform and triangular biphasic waveform;
   an asymmetrical biphasic waveform;
   a double monophasic waveform; and
   a monophasic waveform.
16. The method according to 15, wherein the one or more applied waveforms further comprises a DC offset.
17. The method according to 16, wherein the DC offset is an applied voltage that is sufficient to compensate for each applied electrical stimulation pulse.
18. The method according to any one of 1-17, wherein the method comprises applying the electrical stimulation from two or more channels of a transcutaneous or epidural electrical spinal cord stimulator.
19. The method according to 18, wherein each channel of the transcutaneous or epidural electrical spinal cord stimulator independently applies a different waveform of electrical stimulation.
20. The method according to any one of 18-19, wherein the method comprises applying electrical stimulation to the spinal cord of the subject that comprises a plurality of different waveforms from the same electrode.
21. The method according to 20, wherein each waveform is applied sequentially.
22. The method according to any one of 15-21, wherein each waveform comprises a high frequency component and a low frequency component and wherein the high frequency component provides an analgesic effect on the skin and the low frequency component tunes the spinal cord neurons to achieve the required functional goals.
23. The method according to 22, wherein the high frequency component comprises a frequency of from 1 KHz to 25 KHz.
24. The method according to 23, wherein the high frequency component comprises a frequency of from 5 KHz to 15 KHz.
25. The method according to 23, wherein the high frequency component comprises a frequency of about 10 KHz.
26. The method according to any one of 22-25, wherein the low frequency component comprises a frequency of from 1 Hz to 500 Hz.
27. The method according to 26, wherein the low frequency component comprises a frequency of from 50 Hz to 250 Hz.
28. The method according to 26, wherein the low frequency component comprises a frequency of about 100 Hz.
29. The method according to any one of 1-28, wherein the electrical stimulation is applied at a pulse frequency of 5 Hz or more.
30. The method according to any one of 1-28, wherein the electrical stimulation is applied at a pulse frequency of 25 Hz or more.
31. The method according to any one of 1-28, wherein the electrical stimulation is applied at a pulse frequency of about 30 Hz.
32. The method according to any one of 1-31, wherein the electrical stimulation comprises a pulse amplitude of from 1 mA to 500 mA.
33. The method according to 32, wherein the electrical stimulation comprises a pulse amplitude of from 50 mA to 200 mA.
34. The method according to 32, wherein the electrical stimulation comprises a pulse amplitude of about 100 mA.
35. The method according to any one of 1-34, wherein the electrical stimulation comprises a DC offset amplitude of from 0.1 mA to 10 mA.
36. The method according to 35, wherein the electrical stimulation comprises a DC offset amplitude of from 0.5 mA to 2.5 mA.
37. The method according to 36, wherein the electrical stimulation comprises a DC offset amplitude of about 1.5 mA.
38. The method according to any one of 1-37, wherein the electrical stimulation comprises a pulsed applied DC offset.
39. The method according to any one of 1-38, wherein the electrical stimulation comprises a continuously applied DC offset.
40. The method according to any one of 1-38, wherein the method comprises applying the electrical stimulation to the spinal cord of the subject for 1 hour or more.
41. The method according to 40, wherein the method comprises applying the electrical stimulation to the spinal cord of the subject for 8 to 12 hours per day.
42. The method according to any one of 1-41, wherein the method comprises applying the electrical stimulation to the spinal cord of the subject for 2 to 5 days per week.
43. The method according to any one of 1-42, wherein the method further comprises applying magnetic stimulation to the spine of the subject.
44. The method according to 43, wherein the magnetic stimulation is applied simultaneously with the electrical stimulation.
45. The method according to 43, wherein the magnetic stimulation is applied sequentially with the electrical stimulation.
46. The method according to any one of 1-42, wherein the method further comprises applying mechanical stimulation to the spine of the subject.
47. The method according to 46, wherein the mechanical stimulation is applied simultaneously with the electrical stimulation.
48. The method according to claim 46, wherein the mechanical stimulation is applied sequentially with the electrical stimulation.
49. The method according to any one of 1-42, wherein the method further comprises applying magnetic stimulation and mechanical stimulation to the spine of the subject.
50. The method according to 49, wherein the magnetic stimulation and mechanical stimulation are applied simultaneously with the electrical stimulation.
51. The method according to 49, wherein the magnetic stimulation and mechanical stimulation are applied sequentially with the electrical stimulation.
52. The method according to any one of 1-51, wherein the electrical stimulation is applied to the spinal cord of the subject with a wearable device.
53. The method according to 52, wherein the wearable device is a disposable device.
54. The method according to any one of 52-53, wherein the wearable device comprises a single use battery.
55. The method according to 52, wherein the wearable device comprises a reuseable rechargeable battery.
56. The method according to any one of 52-55, wherein the wearable device comprises a belt or harness worn spring and is configured to ensure hydrogel contact with the skin of the subject.
57. The method according to 56, wherein the springs of the wearable device are configured to provide mechanical and vibrotactile stimulation.
58. The method according to any one of 56-57, wherein the wearable device is configured to route wires under the clothing of the subject and to be connected to a spring based holder.
59. The method according to any one of 1-58, wherein applying the electrical stimulation to the spinal cord of the subject is sufficient to improve vision in the subject.
60. The method according to 59, wherein applying the electrical stimulation to the spinal cord of the subject is sufficient to improve one or more of near sightedness, far sightedness, peripheral vision and visual acuity.
61. The method according to any one of 1-58, wherein applying the electrical stimulation to the spinal cord of the subject is sufficient to improve sense of smell in the subject.
62. The method according to any one of 1-58, wherein applying the electrical stimulation to the spinal cord of the subject is sufficient to improve hearing by the subject.
63. The method according to any one of 1-58, wherein applying the electrical stimulation to the spinal cord of the subject is sufficient to improve voice modulation by the subject.
64. The method according to 63, wherein applying the electrical stimulation to the spinal cord of the subject is sufficient to improve one or more of ability to vocalize, articulation, speaking softly, speaking loudly, and duration of voice modulation by the subject.
65. The method according to any one of 1-58, wherein applying the electrical stimulation to the spinal cord of the subject is sufficient to improve the ability to control swallowing by the subject.
66. The method according to any one of 1-58, wherein applying the electrical stimulation to the spinal cord of the subject is sufficient to improve the ability to control biting by the subject.
67. The method according to any one of 1-58, wherein applying the electrical stimulation to the spinal cord of the subject is sufficient to improve the ability to control sipping by the subject.
68. The method according to any one of 1-58, wherein applying the electrical stimulation to the spinal cord of the subject is sufficient to improve the ability to control movement of the lower jaw by the subject.
69. The method according to any one of 1-58, wherein applying the electrical stimulation to the spinal cord of the subject is sufficient to improve the ability to control movement of the tongue by the subject.
70. The method according to any one of 1-58, wherein applying the electrical stimulation to the spinal cord of the subject is sufficient to improve control of facial muscles by the subject.
71. The method according to 70, wherein applying the electrical stimulation to the spinal cord of the subject is sufficient to improve smiling by the subject.
72. The method according to any one of 1-58, wherein applying the electrical stimulation to the spinal cord of the subject is sufficient to improve sense of taste of the subject.
73. The method according to any one of 1-58, wherein applying the electrical stimulation to the spinal cord of the subject is sufficient to improve movement of the eyeballs by the subject.
74. The method according to any one of 1-58, wherein applying the electrical stimulation to the spinal cord of the subject is sufficient to improve movement of the head and neck by the subject.
75. The method according to any one of 1-58, wherein applying the electrical stimulation to the spinal cord of the subject is sufficient to improve sleep by the subject.
76. The method according to 75, wherein applying the electrical stimulation to the spinal cord of the subject is sufficient to improve one or more of the ability to fall asleep faster, ability to sleep longer without waking up at night and ability to go back to sleep after waking up.
77. The method according to any one of 1-58, wherein applying the electrical stimulation to the spinal cord of the subject is sufficient to reduce or normalize seizure activity in the subject.
78. The method according to any one of 1-58, wherein applying the electrical stimulation to the spinal cord of the subject is sufficient to normalize the resting state of the nervous system of the subject.
79. The method according to any one of 1-58, wherein applying the electrical stimulation to the spinal cord of the subject is sufficient to treat anxiety or depression in the subject.
80. The method according to any one of 1-58, wherein applying the electrical stimulation to the spinal cord of the subject is sufficient to improve vestibular function in the subject selected from the group consisting of vertigo, dizziness, visual disturbance, and imbalance.
81. The method according to any one of 1-58, wherein applying the electrical stimulation to the spinal cord of the subject is sufficient to improve bladder function in the subject selected from the group consisting of increasing bladder capacity, increasing sensation of bladder fullness, reducing urinary incontinence, increasing voluntary control to hold, improving ability to void voluntarily, reducing the use of catheters to empty bladder.
82. The method according to any one of 1-58, wherein applying the electrical stimulation to the spinal cord of the subject is sufficient to improve bowel function in the subject selected from the group consisting of increasing sensation of bowel fullness, reducing fecal incontinence, increasing voluntary control to hold and improving the ability to defecate voluntarily.
83. The method according to any one of 1-58, wherein applying the electrical stimulation to the spinal cord of the subject is sufficient to improve sexual function in the subject selected from the group consisting of improving sensation of urogenital organs, returning the ability to have an erection, increasing lubrication, increasing sensation during erection and penetration, increasing ability for voluntary penetration, increasing ability to sustain erection for longer periods of time and increasing degree of orgasm at climax.
84. The method according to any one of 1-58, wherein applying the electrical stimulation to the spinal cord of the subject is sufficient to increase sperm count, sperm mortality and vitality by the subject.
85. The method according to any one of 1-84, wherein the subject has a condition selected from the group consisting of spinal cord injury, an ischemic brain injury, and a neurodegenerative condition.
86. The method according to 85, wherein the ischemic brain injury is a brain injury from a stroke or acute trauma.
87. The method according to 86, wherein the neurodegenerative condition is selected from the group consisting of stroke, spinal cord injury, Parkinson's disease, Huntington's disease, Alzheimer's disease, amyotrophic lateral sclerosis (ALS), primary lateral sclerosis (PLS), dystonia, hemispherictomy, transverse myelitis, conus medularis injury (lower motor neuron injury), spina bifida, autism, hemispherectomy and cerebral palsy.
88. The method according to any one of 1-84, wherein the subject has a naturally occurring condition selected from group consisting of aging, post-partum, inactivity and post-surgical care.
89. A system for neuromodulating the central nervous system of a subject, the system comprising an electrical stimulator configured to apply electrical stimulation to the spinal cord of a subject in a manner sufficient to maintain voluntary control of physical activity during the electrical stimulation.
90. The system according to 89, wherein the electrical stimulator is configured to apply electrical stimulation to the spinal cord of the subject in a manner sufficient to integrate and reconnect the brain to the spinal cord.
91. The system according to 89, wherein the electrical stimulator is configured to apply electrical stimulation to the spinal cord of the subject in a manner sufficient to maintain voluntary control of one or more of physical motor function, sensory function, vestibular function, cognitive function, autonomic function and sleep activity.
92. The system according to 89, wherein the electrical stimulator is configured to apply electrical stimulation to the spinal cord of the subject in a manner sufficient to maintain voluntary control of one or more of anxiety, depression and mood.
93. The system according to any one of 89-92, wherein the voluntary control of physical activity is maintained by the subject after cessation of the electrical stimulation.
94. The system according to any one of 89-93, wherein the electrical stimulator is configured to apply electrical stimulation to the spinal cord of the subject in a manner sufficient to excite neurons in the brain.
95. The system according to any one of 89-94, wherein the electrical stimulator is configured to apply electrical stimulation to the spinal cord of the subject in a manner sufficient to inhibit neurons in the brain.
96. The system according to any one of 89-95, wherein the electrical stimulator is configured to apply electrical stimulation to the spinal cord of the subject in a manner sufficient to improve voluntary control of joints and muscles of lower extremity, upper extremity, head, neck, facial muscles, sphincters (bladder/bowel), pelvic floor, abdominal, diaphragm, throat muscles, autonomic control of organs, including bladder bowel cardiovascular sexual breathing functions.
97. The system according to any one of 89-96, wherein the electrical stimulator is configured to apply electrical stimulation to the spinal cord of the subject in a manner sufficient to improve sensation of joints and muscles of lower extremity, upper extremity, head, neck, facial muscles, sphincters (bladder/bowel), pelvic floor, abdominal, diaphragm, throat muscles, autonomic control of organs including bladder bowel cardiovascular sexual breathing functions.
98. The system according to any one of 89-97, wherein the electrical stimulator is configured to apply electrical stimulation to the spinal cord of the subject in a manner sufficient to improve vision in the subject.
99. The system according to 98, wherein the electrical stimulator is configured to apply electrical stimulation to the spinal cord of the subject in a manner sufficient to improve one or more of near sightedness, far sightedness, peripheral vision and visual acuity.
100. The system according to any one of 89-99, wherein the electrical stimulator is configured to apply electrical stimulation to the spinal cord of the subject in a manner sufficient to improve sense of smell in the subject.
101. The system according to any one of 89-100, wherein the electrical stimulator is configured to apply electrical stimulation to the spinal cord of the subject in a manner sufficient to improve hearing in the subject.
102. The system according to any one of 89-101, wherein the electrical stimulator is configured to apply electrical stimulation to the spinal cord of the subject in a manner sufficient to improve voice modulation by the subject.
103. The system according to any one of 89-102, wherein the electrical stimulator is configured to apply electrical stimulation to the spinal cord of the subject in a manner sufficient to improve one or more of ability to vocalize, articulation, speaking softly, speaking loudly, and duration of voice modulation by the subject.
104. The system according to any one of 89-103, wherein the electrical stimulator is configured to apply electrical stimulation to the spinal cord of the subject in a manner sufficient to improve the ability to control swallowing by the subject.
105. The system according to any one of 89-104, wherein the electrical stimulator is configured to apply electrical stimulation to the spinal cord of the subject in a manner sufficient to improve the ability to control biting by the subject.
106. The system according to any one of 89-105, wherein the electrical stimulator is configured to apply electrical stimulation to the spinal cord of the subject in a manner sufficient to improve the ability to control sipping by the subject.
107. The system according to any one of 89-106, wherein the electrical stimulator is configured to apply electrical stimulation to the spinal cord of the subject in a manner sufficient to improve the ability to control movement of the lower jaw by the subject.
108. The system according to any one of 89-107, wherein the electrical stimulator is configured to apply electrical stimulation to the spinal cord of the subject in a manner sufficient to improve the ability to control movement of the tongue by the subject.
109. The system according to any one of 89-108, wherein the electrical stimulator is configured to apply electrical stimulation to the spinal cord of the subject in a manner sufficient to improve control of facial muscles by the subject.
110. The system according to any one of 89-109, wherein the electrical stimulator is configured to apply electrical stimulation to the spinal cord of the subject in a manner sufficient to improve smiling by the subject.
111. The system according to any one of 89-110, wherein the electrical stimulator is configured to apply electrical stimulation to the spinal cord of the subject in a manner sufficient to improve sense of taste of the subject.
112. The system according to any one of 89-111, wherein the electrical stimulator is configured to apply electrical stimulation to the spinal cord of the subject in a manner sufficient to improve movement of the eyeballs by the subject.
113. The system according to any one of 89-112, wherein the electrical stimulator is configured to apply electrical stimulation to the spinal cord of the subject in a manner sufficient to improve movement of the head and neck by the subject.
114. The system according to any one of 89-113, wherein the electrical stimulator is configured to apply electrical stimulation to the spinal cord of the subject in a manner sufficient to improve sleep by the subject.
115. The system according to 114, wherein the electrical stimulator is configured to apply electrical stimulation to the spinal cord of the subject in a manner sufficient to improve one or more of the ability to fall asleep faster, ability to sleep longer without waking up at night and ability to go back to sleep after waking up.
116. The system according to any one of 89-115, wherein the electrical stimulator is configured to apply electrical stimulation to the spinal cord of the subject in a manner sufficient to reduce or normalize seizure activity in the subject.
117. The system according to any one of 89-116, wherein the electrical stimulator is configured to apply electrical stimulation to the spinal cord of the subject in a manner sufficient to normalize the resting state of the nervous system of the subject.
118. The system according to any one of 89-117, wherein the electrical stimulator is configured to apply electrical stimulation to the spinal cord of the subject in a manner sufficient to treat anxiety or depression in the subject.
119. The system according to any one of 89-118, wherein the electrical stimulator is configured to apply electrical stimulation to the spinal cord of the subject in a manner sufficient to improve vestibular function in the subject selected from the group consisting of vertigo, dizziness, visual disturbance, and imbalance.
120. The system according to any one of 89-119, wherein the electrical stimulator is configured to apply electrical stimulation to the spinal cord of the subject in a manner sufficient to improve bladder function in the subject selected from the group consisting of increasing bladder capacity, increasing sensation of bladder fullness, reducing urinary incontinence, increasing voluntary control to hold, improving ability to void voluntarily, reducing the use of catheters to empty bladder.
121. The system according to any one of 89-120, wherein the electrical stimulator is configured to apply electrical stimulation to the spinal cord of the subject in a manner sufficient to improve bowel function in the subject selected from the group consisting of increasing sensation of bowel fullness, reducing fecal incontinence, increasing voluntary control to hold and improving the ability to defecate voluntarily.
122. The system according to any one of 89-121, wherein the electrical stimulator is configured to apply electrical stimulation to the spinal cord of the subject in a manner sufficient to improve sexual function in the subject selected from the group consisting of improving sensation of urogenital organs, returning the ability to have an erection, increasing lubrication, increasing sensation during erection and penetration, increasing ability for voluntary penetration, increasing ability to sustain erection for longer periods of time and increasing degree of orgasm at climax.
123. The system according to 122, wherein the electrical stimulator is configured to apply electrical stimulation to the spinal cord of the subject in a manner sufficient to increase sperm count, sperm mortality and vitality by the subject.
124. The system according to any one of 89-123, wherein the electrical stimulator is configured to apply electrical stimulation to the spinal cord of the subject in a manner sufficient to retrain the spinal neural network of the central nervous system of the subject.
125. The system according to any one of 89-124, wherein the electrical stimulator is configured to apply electrical stimulation to the spinal cord of the subject in a manner sufficient to reconnect the spinal neural network with the brain of the subject.
126. The system according to any one of 89-125, wherein the electrical stimulator is configured to apply electrical stimulation to the spinal cord of the subject in a manner sufficient to enhance voluntary control of physical activity by the subject.
127. The system according to any one of 89-126, wherein the electrical stimulator is configured to apply electrical stimulation to the spinal cord of the subject in a manner sufficient to increase ascending neural signals to the brain of the subject.
128. The system according to any one of 89-127, wherein the electrical stimulator comprises one or more channels configured to apply at least one waveform selected from the group consisting of:
   one or more a trapezoidal monophasic waveform and a trapezoidal biphasic waveform;
   one or more of a triangular monophasic waveform and triangular biphasic waveform;
   an asymmetrical biphasic waveform;
   a double monophasic waveform; and
   a monophasic waveform.
129. The system according to 128, wherein the electrical stimulator is configured to apply the one or more waveforms with a DC offset.
130. The system according to 129, wherein the DC offset is an applied voltage that is sufficient to compensate for each applied electrical stimulation pulse.
131. The system according to any one of 128-130, wherein each waveform is applied from different channels of the electrical stimulator.
132. The system according to any one of 128-131, wherein each waveform comprises a high frequency component and a low frequency component and wherein the high frequency component provides an analgesic effect on the skin and the low frequency component tunes the spinal cord neurons to achieve the required functional goals.
133. The system according to 132, wherein the high frequency component comprises a frequency of from 1 KHz to 25 KHz.
134. The system according to 133, wherein the high frequency component comprises a frequency of from 5 KHz to 15 KHz.
135. The system according to 134, wherein the high frequency component comprises a frequency of about 10 KHz.
136. The system according to any one of 132-135, wherein the low frequency component comprises a frequency of from 1 Hz to 500 Hz.
137. The system according to 136, wherein the low frequency component comprises a frequency of from 50 Hz to 250 Hz.
138. The system according to 137, wherein the low frequency component comprises a frequency of about 100 Hz.
139. The system according to any one of 89-138, wherein the electrical stimulator is configured to apply the electrical stimulation at a pulse frequency of 5 Hz or more.
140. The system according to any one of 89-138, wherein the electrical stimulator is configured to apply the electrical stimulation at a pulse frequency of 25 Hz or more.
141. The system according to any one of 89-138, wherein the electrical stimulator is configured to apply the electrical stimulation at a pulse frequency of about 30 Hz.
142. The system according to any one of 89-141, wherein the electrical stimulator is configured to apply electrical stimulation having a pulse amplitude of from 1 mA to 500 mA.
143. The system according to any one of 89-142, wherein the electrical stimulator is configured to apply electrical stimulation having a pulse amplitude of from 50 mA to 200 mA.
144. The system according to 143, wherein the electrical stimulator is configured to apply electrical stimulation having a pulse amplitude of about 100 mA.
145. The system according to any one of 89-144, wherein the electrical stimulator is configured to apply electrical stimulation having a DC offset amplitude of from 0.1 mA to 10 mA.
146. The system according to 145, wherein the electrical stimulator is configured to apply electrical stimulation having a DC offset amplitude of from 0.5 mA to 2.5 mA.
147. The system according to 146, wherein the electrical stimulator is configured to apply electrical stimulation having a DC offset amplitude of about 1.5 mA.
148. The system according to any one of 89-147, wherein the electrical stimulator is configured to apply a pulsed DC offset.
149. The system according to 148, wherein the electrical stimulator is configured to apply a continuously applied DC offset.
150. The system according to any one of 89-149, wherein the electrical stimulator is integrated into clothing.
151. The system according to any one of 89-149 wherein the electrical stimulator is integrated into a chair.
152. The system according to any one of 89-149, wherein the electrical stimulator is integrated into a wearable device.
153. The system according to 152, wherein the wearable device is a disposable device.
154. The system according to any one of 152-153, wherein the wearable device comprises a single use battery.
155. The system according to 154, wherein the wearable device comprises a reuseable rechargeable battery.
156. The system according to any one of 152-155, wherein the wearable device comprises a belt or harness worn spring and is configured to ensure hydrogel contact with the skin of the subject.
157. The system according to 156, wherein the springs of the wearable device are configured to provide mechanical and vibrotactile stimulation.
158. The system according to any one of 152-157, wherein the wearable device is configured to route wires under the clothing of the subject and to be connected to a spring based holder.
159. The system according to any one of 89-158, further comprising a mechanical stimulator configured to apply mechanical stimulation to the spine of a subject.
160. The system according to 159, wherein the mechanical stimulator is configured to apply mechanical stimulation to the spine of the subject simultaneously with the electrical stimulation.
161. The system according to 160, wherein the mechanical stimulator is configured to apply mechanical stimulation to the spine of the subject sequentially with the electrical stimulation.
162. The system according to any one of 89-161, further comprising a magnetic stimulator configured to apply magnetic stimulation to the spine of a subject.
163. The system according to 162, wherein the magnetic stimulator is configured to apply magnetic stimulation to the spine of the subject simultaneously with the electrical stimulation.
164. The system according to 162, wherein the magnetic stimulator is configured to apply magnetic stimulation to the spine of the subject sequentially with the electrical stimulation.
165. A method of neuromodulation in a subject having delayed or abnormal brain development, the method comprising applying electrical stimulation to the spinal cord of the subject in a manner sufficient to induce neuroplasticity of the brain and spinal cord neural network of the subject.
166. The method according to 165, wherein the neuromodulation is sufficient to provide for acceleration of developmental milestones, initially delayed due to delayed or abnormal development.
167. The method according to 165, wherein the subject is diagnosed with Fragile X syndrome, Trisomy 21, a chromosomal abnormality, tuberous sclerosis, neurofibromatosis, phenylketonuria, a myopathy, Hydrocephalus, Lissencephaly, spina bifida, autism spectrum disorder, fetal alcohol syndrome, Landau Kleffner syndrome or cerebral palsy.
168. The method according to 167, wherein the subject exhibits symptoms of or is diagnosed with cerebral palsy.
169. The method according to any one of 165-168, wherein the subject suffers from non-traumatic brain damage.
170. The method according to 169, wherein the subject suffers from non-traumatic brain damage that occurred within about 2-3 years from birth.
171. The method according to any one of 165-170, where the neuromodulation comprises applying the electrical stimulation at a frequency and amplitude sufficient to activate one or more of the sensory neurons and the interneurons of the spinal cord neural network.
172. The method according to 171, wherein the neuromodulation comprises applying the electrical stimulation at a frequency and amplitude sufficient to activate sensory neurons of the spinal cord neural network.
173. The method according to 171, wherein the neuromodulation comprises applying the electrical stimulation at a frequency and amplitude sufficient to activate interneurons of the spinal cord neural network.
174. The method according to any one of 165-173, wherein the neuromodulation does not directly activate the motor neurons of the spinal cord neural network.
175. The method according to any one of 172-174, wherein the neuromodulation comprises applying the electrical stimulation at a frequency and amplitude sufficient to:
   activate one or more of the sensory neurons and the interneurons of the spinal cord neural network; and
   not directly activate the motor neurons of the spinal cord neural network.
176. The method according to any one of 172-174, wherein the neuromodulation comprises applying the electrical stimulation at a frequency and amplitude which activates interneurons of the spinal cord sufficient to facilitate signal conduction to motor neurons.
177. The method according to 176, wherein the signal conduction to motor neurons is sufficient to provide for voluntary muscle control by the subject.
178. The method according to 177, wherein voluntary muscle control comprises one or more of:
   activating one or more muscle groups;
   inhibiting activity by one or more muscle groups; and
   having no impact on one or more muscle groups.
179. The method according to any one of 177-178, wherein voluntary muscle control comprises the absence or reduced presence of spasticity exhibited by the subject.
180. The method according to any one of 177-179, wherein voluntary control comprises the absence or reduced presence of one or more of reflexes, floppiness or involuntary movements exhibited by the subject.
181. The method according to any one of 177-180, wherein voluntary muscle control comprises the absence or reduced presence of co-contraction of anagonistic muscle activity exhibited by the subject.
182. The method according to 165-181, wherein the neuromodulation comprises applying the electrical stimulation in a manner sufficient to enable and learn a non-patterned, non-repetitive, stochastic motor response by the subject.
183. The method according to claim 182, wherein the neuromodulation comprises applying the electrical stimulation in a manner sufficient to enable a voluntary motor initiation response by the subject.
184. The method according to any one of 182-183, wherein the neuromodulation comprises applying the electrical stimulation in a manner sufficient to enable voluntary control of trunk alignment by the subject.
185. The method according to any one of 182-184, wherein the neuromodulation comprises applying the electrical stimulation in a manner sufficient to enable voluntary control of posture by the subject.
186. The method according to any one of 182-185, wherein the neuromodulation comprises applying the electrical stimulation in a manner sufficient to enable voluntary control during dynamic standing and stepping by the subject.
187. The method according to any one of 182-186, wherein the neuromodulation comprises applying the electrical stimulation in a manner sufficient to enable voluntary control of the center of mass by the subject.
188. The method according to 187, wherein voluntary control of the center of mass comprises maintaining the center of mass of the subject over a base of support.
189. The method according to any one of 182-188, wherein the neuromodulation comprises applying the electrical stimulation in a manner sufficient to provide for identifying and maintaining midline orientation by the subject.
190. The method according to 189, wherein the neuromodulation comprises applying the electrical stimulation in a manner sufficient to provide for identifying midline orientation with bilateral hand and arm activities.
191. The method according to 190, wherein the bilateral hand and arm activities comprise one or more of clapping and jumping jacks.
192. The method according to any one of 182-191, wherein the neuromodulation comprises applying the electrical stimulation in a manner sufficient to provide for maintaining weight bearing standing by the subject.
193. The method according to 192, wherein the neuromodulation comprises applying the electrical stimulation in a manner sufficient to provide for maintaining weight bearing standing with heels on the ground.
194. The method according to any one of 182-191, wherein the neuromodulation comprises applying the electrical stimulation in a manner sufficient to provide for maintaining weight bearing sitting balance by the subject.
195. The method according to 194, wherein the neuromodulation comprises applying the electrical stimulation in a manner sufficient to provide for maintaining weight bearing sitting balance with head over ischial tuberosities by the subject.
196. The method according to any one of 182-195, wherein the neuromodulation comprises applying the electrical stimulation in a manner sufficient to provide for maintaining a predetermined balance and posture by the subject.
197. The method according to any one of 182-196, wherein the neuromodulation comprises applying the electrical stimulation to enable voluntary control by the subject sufficient to perform one or more of: head control, stepping, climbing, upright sitting, shifting weight, control movement or alignment of the trunk, dynamic standing with postural or weight adjustment, transition from sitting to standing, transition from stand to walk, walk to run, increasing and decreasing speed of walking, transition from standing to sitting, crawling, proning, rolling, nodding and gesturing.
198. The method according to any one of 182-197, wherein the neuromodulation comprises applying the electrical stimulation to enable voluntary control by the subject during standing and walking sufficient to perform one or more of: maintaining the hips back, not leaning forward, maintaining contact of the heels with the ground, not rotating the hips, knee or ankles internally or externally.
199. The method according to any one of 165-198, wherein the neuromodulation comprises applying the electrical stimulation at a frequency and amplitude that increases the visual acuity of the subject.
200. The method according to any one of 165-199, wherein the neuromodulation comprises increasing processing of proprioception in the brain and spinal cord.
201. The method according to any one of 165-200, wherein the neuromodulation comprises increasing processing of descending voluntary signals from the brain to the spinal cord of the subject.
202. The method according to any one of 200-201, wherein the neuromodulation comprises increasing proprioception in the brain and spinal cord of the subject in a manner sufficient to facilitate sense of touch.
203. The method according to any one of 200-201, wherein the neuromodulation comprises increasing proprioception in the brain and spinal cord of the subject in a manner sufficient to facilitate or improve judgement of distance by the subject.
204. The method according to any one of 200-201, wherein the neuromodulation comprises increasing proprioception in the brain and spinal cord of the subject in a manner sufficient to facilitate or improve judgement of object size by the subject.
205. The method according to any one of 200-201, wherein the neuromodulation comprises increasing proprioception in the brain and spinal cord of the subject in a manner sufficient to improve visual tracking by the subject.
206. The method according to 205, wherein the neuromodulation comprises increasing proprioception in the brain and spinal cord of the subject in a manner sufficient to improve peripheral visual tracking by the subject.
207. The method according to 205, wherein the neuromodulation comprises increasing proprioception in the brain and spinal cord of the subject in a manner sufficient to improve cross-midline visual tracking by the subject.
208. The method according to any one of 204-207, wherein the neuromodulation comprises increasing proprioception in the brain and spinal cord of the subject in a manner sufficient to change cortical visual impairment of the subject.
209. The method according to 200-208, wherein the neuromodulation comprises increasing proprioception in the brain and spinal cord of the subject in a manner sufficient to improve visual focus of the subject.
210. The method according to 209, wherein the neuromodulation comprises increasing proprioception in the brain and spinal cord of the subject in a manner sufficient to facilitate or improve judgement of falling by the subject.
211. The method according to 209, wherein the neuromodulation comprises increasing proprioception in the brain and spinal cord of the subject in a manner sufficient to prevent involuntary falling by the subject.
212. The method according to 200-211, wherein the neuromodulation comprises increasing proprioception in the brain and spinal cord of the subject in a manner sufficient to provide for voluntary control of two or more of the head, hands and arm, trunk, and legs in a synchronized manner.
213. The method according to 212, wherein the neuromodulation comprises increasing proprioception in the brain and spinal cord of the subject in a manner sufficient to provide for aligning two or more of the head, hands and arms, trunk, and legs.
214. The method according to any one of 212-213, wherein the neuromodulation comprises increasing proprioception in the brain and spinal cord of the subject in a manner sufficient to provide for maintaining two or more of the head, hands and arms, trunk, and legs in alignment with the center of mass directly over the base of support while walking.
215. The method according to any one of 165-214, wherein the neuromodulation comprises increasing self-motivation, excitement and engagement in activities by the subject.
216. The method according to any one of 165-214, wherein the neuromodulation comprises increasing self-initiated communication.
217. The method according to 216, wherein the self-initiated communication comprises non-verbal communication or verbal communication.
218. The method according to 217, wherein the non-verbal communication comprises one or more of gestures, eye tracking, eye movement, head nodding, smiling, crying and laughing.
219. The method according to any one of 165-218, wherein neuromodulation comprises increasing proprioception in the brain and spinal cord of the subject in a manner sufficient to increase spatial recognition by the subject.
220. The method according to 219, wherein spatial recognition comprises informing the subject as to where one or more parts of the body are in space.
221. The method according to 165-220, wherein neuromodulation comprises increasing proprioception in the brain and spinal cord of the subject in when the subject is in prone position, the center of mass is in the pelvis with the ground reaction forces acting on the anterior surface of the body.
222. The method according to any one of 165-220, wherein neuromodulation comprises increasing proprioception in the brain and spinal cord of the subject when the subject is in sitting position, the center of mass is directly over the ischial tuberosities.
223. The method according to any one of 165-220, wherein neuromodulation comprises increasing proprioception in the brain and spinal cord of the subject when the subject is in quadruped position, the center of mass is in between the knees and hands and the ground reaction forces are at the heels of the hands, the knees and the feet.
224. The method according to any one of 165-220, wherein neuromodulation comprises increasing proprioception in the brain and spinal cord of the subject when the subject is standing on a two-leg position, the center of mass is directly in between the two feet, over the heels.
225. The method according to any one of 165-220, wherein neuromodulation comprises increasing proprioception in the brain and spinal cord of the subject when the subject is standing on a one-leg position, the center of mass is directly over the heel in contact with the ground.
226. The method according to any one of 165-225, where the neuromodulation comprises applying the electrical stimulation at frequency and amplitude sufficient to improve intellectual disabilities of the subject.
227. The method according to any one of 165-226, the neuromodulation is sufficient to reduce a long-term complication in the subject.
228. The method according to 227, wherein the long-term complication comprises one or more of contractures, joint displacement, depression, social anxiety, heart and lung diseases, osteoarthritis and osteoporosis.
229. The method according to any one of 165-228, wherein the method further comprises of providing one or more of verbal and tactile queues to the subject.
230. The method according to 229, wherein the verbal or tactile queues are sufficient to allow the subject to voluntarily correct an error.
231. The method according to 230, wherein the method further comprises providing physical assistance to the subject only after the subject has committed an error.
232. The method according to 231, wherein assistance is not provided during or prior to the error being committed.
233. The method according to any one of 165-232, wherein the method comprises the subject maintaining the head in an upright position with the eyes parallel to the horizontal plane for appropriate visual input.
234. The method according to any one of 165-233, wherein the method comprises the subject maintaining the head, trunk, pelvis and ischial tuberosities in alignment with the center of mass directly over the ischial tuberosities.
235. The method according to any one of 165-234, wherein the method comprises of maintaining the hands and arms free to explore and interact with a surrounding space and further increase proprioceptive information from an upper extremity.
236. The method according to any one of 165-235, wherein the method comprises movement or activity by the subject in a non-repetitive or non-patterned manner.
237. The method according to any one of 165-236, wherein the method comprises of generating one or more of weight shifts, postural adjustments, external support and changes in alignment by movement of the hip and pelvis.
238. The method according to 237, wherein the subject does not move the shoulders and ankles.
239. The method according to any one of 165-238, wherein the method comprises non-invasively applying the electrical stimulation.
240. The method according to any one of claim 239, wherein the method comprises applying the electrical stimulation with one or more of: a powered exoskeleton device, a powered or active orthosis, a passive orthosis, a wearable orthosis, a soft exoskeleton device, a hip orthosis, a knee orthosis, a head orthosis, an ankle orthosis, a body weight support device, a stand frame, a wheelchair, a set of crutches and a walker.
241. A method of neuromodulation in a subject having a spinal cord injury, the method comprising applying electrical stimulation to the spinal cord of the subject acutely after the spinal cord injury in a manner sufficient to induce a plastic change in one or more of the brain and spinal cord.
242. The method according to 241, wherein the electrical stimulation is applied to the spinal cord of the subject 6 months or less after the spinal cord injury.
243. The method according to 241, wherein the electrical stimulation is applied to the spinal cord of the subject 3 months or less after the spinal cord injury.
244. The method according to 241, wherein the electrical stimulation is applied to the spinal cord of the subject 6 weeks or less after the spinal cord injury.
245. The method according to any one of 241-244, wherein the electrical stimulation is applied to the spinal cord of the subject before post-injury innervation.
246. The method according to any one of 241-245, wherein the electrical stimulation is applied to the spinal cord of the subject before post-injury hyperinnervation.
247. The method according to any one of 241-245, wherein the electrical stimulation is applied to the spinal cord of the subject during post-injury spinal shock.
248. The method according to any one of 241-247, wherein the electrical stimulation is applied to the spinal cord of the subject in a manner sufficient to prevent aberrant connections in the brain and spinal cord of the subject.
249. The method according to 248, wherein the neuromodulation is sufficient to prevent aberrant connections in the brain and spinal cord of the subject during post-injury spinal shock.
250. The method according to any one of 241-249, wherein the electrical stimulation is applied to the spinal cord of the subject in a manner sufficient to reduce or prevent scar tissue formation at the site of the spinal cord injury.
251. The method according to any one of 241-249, wherein the electrical stimulation is applied to the spinal cord of the subject in a manner sufficient to increase blood flow to the site of the spinal cord injury.
252. The method according to any one of 241-249, wherein the electrical stimulation is applied to the spinal cord of the subject in a manner sufficient to increase blood flow to a site along the spinal cord that is above the spinal cord injury and/or to a site along the brain that is above the spinal cord injury.
253. The method according to any one of 241-249, wherein the electrical stimulation is applied to the spinal cord of the subject in a manner sufficient to increase blood flow to a site along the spinal cord that is below the spinal cord injury.
254. The method according to any one of 241-253, wherein the electrical stimulation is applied to the spinal cord of the subject in a manner sufficient to delay or prevent detrusor overactivity in the subject.
255. The method according to 254, wherein the electrical stimulation is applied to the spinal cord of the subject in a manner sufficient to delay or prevent detrusor overactivity in the subject during post-injury spinal shock.
256. The method according to any one of 241-255, wherein the electrical stimulation is applied to the spinal cord of the subject in a manner sufficient to reduce spasticity of the detrusor and urethral sphincter.
257. The method according to any one of 254-256, wherein the neuromodulation is sufficient to increase voluntary control of the urethral sphincter in the subject to allow contraction and relaxation of the muscle based on whether the subject intends to store urine or void urine.
258. The method according to 257, wherein the subject is capable of one or more of storing urine in the bladder and voluntarily voiding the urine from the bladder during electrical stimulation.
259. The method according to 258, wherein the subject is capable of one or more of storing urine in the bladder and voluntarily voiding the urine from the bladder in the absence of active electrical stimulation.
260. The method according to any one of 256-259, wherein the subject is capable of one or more of voluntarily contracting the detrusor and simultaneously relaxing the urethral sphincter in the absence of active electrical stimulation.
261. The method according to any one of 256-260, wherein neuromodulation is sufficient to increase sense by the subject of bladder fullness.
262. The method according to any one of 256-261, wherein neuromodulation is sufficient to increase bladder capacity of the subject.
263. The method according to any one of 256-262, wherein the electrical stimulation is applied to the spinal cord of the subject in a manner sufficient to facilitate voluntary delayed voiding contraction.
264. The method according to 263, wherein voiding contraction is delayed by an applied voluntary increase in urethral pressure by the subject.
265. The method according to 264, wherein the voluntary increase in urethral pressure is applied in a sustained manner.
266. The method according to 264, wherein the voluntary increase in urethral pressure is not applied in a spastic manner.
267. The method according to any one of 256-266, wherein the electrical stimulation is applied to the spinal cord of the subject in a manner sufficient to facilitate voluntary detrusor contraction.
268. The method according to 267, wherein the electrical stimulation is applied to the spinal cord of the subject in a manner sufficient to facilitate a decrease in urethral pressure in response to voluntary detrusor contraction.
269. The method according to any one of 256-268, wherein the frequency of voluntary voids increases in the absence of active stimulation.
270. The method according to any one of 256-268, wherein the volume of voluntary voids increases in the absence of active stimulation.
271. The method according to any one of 256-268, wherein the number of catheters used decreases in the absence of active stimulation.
272. The method according to any one of 241-271, wherein the electrical stimulation is applied to the spinal cord of the subject in a manner sufficient to increase one or more of voluntary initiation and voluntary completion of bowel movement by the subject.
273. The method according to any one of 241-272, wherein neuromodulation is sufficient to increase sense by the subject of bowel fullness.
274. The method according to any one of 272-273, wherein the electrical stimulation is applied to the spinal cord of the subject in a manner sufficient to facilitate voluntary contractions of one or more of the anus, rectum and other bowel sections.
275. The method according to any one of 241-274, wherein the electrical stimulation is applied to the spinal cord of the subject in a manner sufficient to increase one or more voluntary sexual function by the subject.
276. The method according to 275, wherein the electrical stimulation is applied to the spinal cord of the subject in a manner sufficient to facilitate voluntary generation of psychogenic erection by the subject.
277. The method according to 275, wherein the electrical stimulation is applied to the spinal cord of the subject in a manner sufficient to facilitate voluntary generation of reflex erection by the subject.
278. The method according to 275, wherein the electrical stimulation is applied to the spinal cord of the subject in a manner sufficient to facilitate voluntary ejaculation by the subject.
279. The method according to 275, wherein the electrical stimulation is applied to the spinal cord of the subject in a manner sufficient to facilitate performance of sexual intercourse by the subject.
280. The method according to 275, wherein the electrical stimulation is applied to the spinal cord of the subject in a manner sufficient to increase or improve sense of sexual function by the subject.
281. A system for neuromodulating the central nervous system of a subject, the system comprising an electrical stimulator configured to apply electrical stimulation to the spinal cord of a subject in a manner sufficient to induce neuroplasticity of the brain and spinal cord neural network of the subject.
282. The system according to 281, wherein the neuromodulation is sufficient to provide for acceleration of developmental milestones, initially delayed due to delayed or abnormal development.
283. The system according to any one of 281-282, wherein the electrical stimulator is configured to apply electrical stimulation at a frequency and amplitude sufficient to activate one or more of the sensory neurons and the interneurons of the spinal cord neural network.
284. The system according to 283, wherein the electrical stimulator is configured to apply electrical stimulation at a frequency and amplitude sufficient to activate sensory neurons of the spinal cord neural network.
285. The system according to 283, wherein the electrical stimulator is configured to apply electrical stimulation at a frequency and amplitude sufficient to activate interneurons of the spinal cord neural network.
286. The system according to any one of 281-285, wherein the electrical stimulator is configured to apply electrical stimulation that does not directly activate the motor neurons of the spinal cord neural network.
287. The system according to any one of 281-286, wherein the electrical stimulator is configured to apply electrical stimulation at a frequency and amplitude sufficient to:
   activate one or more of the sensory neurons and the interneurons of the spinal cord neural network; and
   not directly activate the motor neurons of the spinal cord neural network.
288. The system according to any one of 284-287, wherein the electrical stimulator is configured to apply electrical stimulation at a frequency and amplitude which activates interneurons of the spinal cord sufficient to facilitate signal conduction to motor neurons.
289. The system according to 288, wherein the signal conduction to motor neurons is sufficient to provide for voluntary muscle control by the subject.
290. The system according to 289, wherein voluntary muscle control comprises one or more of:
   activating one or more muscle groups;
   inhibiting activity by one or more muscle groups; and
   having no impact on one or more muscle groups.
291. The system according to any one of 289-290, wherein voluntary muscle control comprises the absence or reduced presence of spasticity exhibited by the subject.
292. The system according to any one of 289-291, wherein voluntary control comprises the absence or reduced presence of one or more of reflexes, floppiness or involuntary movements exhibited by the subject.
293. The system according to any one of 289-292, wherein voluntary muscle control comprises the absence or reduced presence of co-contraction of anagonistic muscle activity exhibited by the subject.
294. The system according to any one of 281-293, wherein the electrical stimulator is configured to apply electrical stimulation in a manner sufficient to enable and learn a non-patterned, non-repetitive, stochastic motor response by the subject.
295. The system according to 294, wherein the electrical stimulator is configured to apply electrical stimulation in a manner sufficient to enable a voluntary motor initiation response by the subject.
296. The system according to any one of claims 294-295, wherein the electrical stimulator is configured to apply electrical stimulation in a manner sufficient to enable voluntary control of trunk alignment by the subject.
297. The system according to any one of 294-296, wherein the electrical stimulator is configured to apply electrical stimulation in a manner sufficient to enable voluntary control of posture by the subject.
298. The system according to any one of 294-297, wherein the electrical stimulator is configured to apply electrical stimulation in a manner sufficient to enable voluntary control during dynamic standing and stepping by the subject.
299. The system according to any one of 294-298, wherein the electrical stimulator is configured to apply electrical stimulation in a manner sufficient to enable voluntary control of the center of mass by the subject.
300. The system according to 299, wherein voluntary control of the center of mass comprises maintaining the center of mass of the subject over a base of support.
301. The system according to any one of 294-300, wherein the electrical stimulator is configured to apply electrical stimulation in a manner sufficient to provide for identifying and maintaining midline orientation by the subject.
302. The system according to 301, wherein the electrical stimulator is configured to apply electrical stimulation in a manner sufficient to provide for identifying midline orientation with bilateral hand and arm activities.
303. The system according to 302, wherein the bilateral hand and arm activities comprise one or more of clapping and jumping jacks.
304. The system according to any one of 294-303, wherein the electrical stimulator is configured to apply electrical stimulation in a manner sufficient to provide for maintaining weight bearing standing by the subject.
305. The system according to 304, wherein the electrical stimulator is configured to apply electrical stimulation in a manner sufficient to provide for maintaining weight bearing standing with heels on the ground.
306. The system according to any one of 294-305, wherein the electrical stimulator is configured to apply electrical stimulation in a manner sufficient to provide for maintaining weight bearing sitting balance by the subject.
307. The system according to 306, wherein the electrical stimulator is configured to apply electrical stimulation in a manner sufficient to provide for maintaining weight bearing sitting balance with head over ischial tuberosities by the subject.
308. The system according to any one of 294-307, wherein the electrical stimulator is configured to apply electrical stimulation in a manner sufficient to provide for maintaining a predetermined balance and posture by the subject.
309. The system according to any one of 294-308, wherein the electrical stimulator is configured to apply electrical stimulation to enable voluntary control by the subject sufficient to perform one or more of: head control, stepping, climbing, upright sitting, shifting weight, control movement or alignment of the trunk, dynamic standing with postural or weight adjustment, transition from sitting to standing, transition from stand to walk, walk to run, increasing and decreasing speed of walking, transition from standing to sitting, crawling, proning, rolling, nodding and gesturing.
310. The system according to any one of 294-309, wherein the electrical stimulator is configured to apply electrical stimulation to enable voluntary control by the subject during standing and walking sufficient to perform one or more of: maintaining the hips back, not leaning forward, maintaining contact of the heels with the ground, not rotating the hips, knee or ankles internally or externally.
311. The system according to any one of 281-310, wherein the electrical stimulator is configured to apply electrical stimulation at a frequency and amplitude that increases the visual acuity of the subject.
312. The system according to any one of 281-310, wherein the electrical stimulator is configured to apply electrical stimulation at a frequency and amplitude that increases processing of proprioception in the brain and spinal cord.
313. The system according to any one of 281-312, wherein the electrical stimulator is configured to apply electrical stimulation to increase processing of descending voluntary signals from the brain to the spinal cord of the subject.
314. The system according to any one of 312-313, wherein the electrical stimulator is configured to apply electrical stimulation to increase proprioception in the brain and spinal cord of the subject in a manner sufficient to facilitate sense of touch.
315. The system according to any one of 312-314, wherein the electrical stimulator is configured to apply electrical stimulation to increase proprioception in the brain and spinal cord of the subject in a manner sufficient to facilitate or improve judgement of distance by the subject.
316. The system according to any one of 312-315, wherein the electrical stimulator is configured to apply electrical stimulation to increase proprioception in the brain and spinal cord of the subject in a manner sufficient to facilitate or improve judgement of object size by the subject.
317. The system according to any one of 312-316, wherein the electrical stimulator is configured to apply electrical stimulation to increase proprioception in the brain and spinal cord of the subject in a manner sufficient to improve visual tracking by the subject.
318. The system according to 317, wherein the electrical stimulator is configured to apply electrical stimulation to increase proprioception in the brain and spinal cord of the subject in a manner sufficient to improve peripheral visual tracking by the subject.
319. The system according to 317, wherein the electrical stimulator is configured to apply electrical stimulation to increase proprioception in the brain and spinal cord of the subject in a manner sufficient to improve cross-midline visual tracking by the subject.
320. The system according to any one of 312-319, wherein the electrical stimulator is configured to apply electrical stimulation to increase proprioception in the brain and spinal cord of the subject in a manner sufficient to change cortical visual impairment of the subject.
321. The system according to any one of 312-319, wherein the electrical stimulator is configured to apply electrical stimulation to increase proprioception in the brain and spinal cord of the subject in a manner sufficient to improve visual focus of the subject.
322. The system according to 321, wherein the electrical stimulator is configured to apply electrical stimulation to increase proprioception in the brain and spinal cord of the subject in a manner sufficient to facilitate or improve judgement of falling by the subject.
323. The system according to 321, wherein the electrical stimulator is configured to apply electrical stimulation to increase proprioception in the brain and spinal cord of the subject in a manner sufficient to prevent involuntary falling by the subject.
324. The system according to any one of 312-319, wherein the electrical stimulator is configured to apply electrical stimulation to increase proprioception in the brain and spinal cord of the subject in a manner sufficient to provide for voluntary control of two or more of the head, hands and arm, trunk, and legs in a synchronized manner.
325. The system according to 324, wherein the electrical stimulator is configured to apply electrical stimulation to increase proprioception in the brain and spinal cord of the subject in a manner sufficient to provide for aligning two or more of the head, hands and arms, trunk, and legs.
326. The system according to any one of 324-325, wherein the electrical stimulator is configured to apply electrical stimulation to increase proprioception in the brain and spinal cord of the subject in a manner sufficient to provide for maintaining two or more of the head, hands and arms, trunk, and legs in alignment with the center of mass directly over the base of support while walking.
327. The system according to any one of 281-326, wherein the electrical stimulator is configured to apply electrical stimulation in a manner sufficient to increase self-motivation, excitement and engagement in activities by the subject.
328. The system according to any one of 281-327, wherein the electrical stimulator is configured to apply electrical stimulation in a manner sufficient to increase self-initiated communication.
329. The system according to 328, wherein the self-initiated communication comprises non-verbal communication or verbal communication.
330. The system according to 329, wherein the non-verbal communication comprises one or more of gestures, eye tracking, eye movement, head nodding, smiling, crying and laughing.
331. The system according to any one of 281-326, wherein the electrical stimulator is configured to apply electrical stimulation to increase proprioception in the brain and spinal cord of the subject in a manner sufficient to increase spatial recognition by the subject.
332. The system according to 331, wherein spatial recognition comprises informing the subject as to where one or more parts of the body are in space.
333. The system according to any one of 281-332, wherein the electrical stimulator is configured to apply electrical stimulation to increase proprioception in the brain and spinal cord of the subject in when the subject is in prone position, the center of mass is in the pelvis with the ground reaction forces acting on the anterior surface of the body.
334. The system according to any one of 281-332, wherein the electrical stimulator is configured to apply electrical stimulation to increase proprioception in the brain and spinal cord of the subject when the subject is in sitting position, the center of mass is directly over the ischial tuberosities.
335. The system according to any one of 281-332, wherein the electrical stimulator is configured to apply electrical stimulation to increase proprioception in the brain and spinal cord of the subject when the subject is in quadruped position, the center of mass is in between the knees and hands and the ground reaction forces are at the heels of the hands, the knees and the feet.
336. The system according to any one of 281-332, wherein the electrical stimulator is configured to apply electrical stimulation to increase proprioception in the brain and spinal cord of the subject when the subject is standing on a two-leg position, the center of mass is directly in between the two feet, over the heels.
337. The system according to any one of 281-332, wherein the electrical stimulator is configured to apply electrical stimulation to increase proprioception in the brain and spinal cord of the subject when the subject is standing on a one-leg position, the center of mass is directly over the heel in contact with the ground.
338. The system according to any one of 281-337, wherein the electrical stimulator is configured to apply electrical stimulation at frequency and amplitude sufficient to improve intellectual disabilities of the subject.
339. The system according to any one of 281-337, wherein the electrical stimulator is configured to apply electrical stimulation in a manner sufficient to reduce a long-term complication in the subject.
340. The system according to 339, wherein the long-term complication comprises one or more of contractures, joint displacement, depression, social anxiety, heart and lung diseases, osteoarthritis and osteoporosis.
341. The system according to any one of 281-340, wherein the system comprises one or more of: a powered exoskeleton device, a powered or active orthosis, a passive orthosis, a wearable orthosis, a soft exoskeleton device, a hip orthosis, a knee orthosis, a head orthosis, an ankle orthosis, a body weight support device, a stand frame, a wheelchair, a set of crutches and a walker.
342. The system according to any one of 281-341, wherein the electrical stimulator comprises one or more channels configured to apply at least one waveform selected from the group consisting of:
   one or more a trapezoidal monophasic waveform and a trapezoidal biphasic waveform;
   one or more of a triangular monophasic waveform and triangular biphasic waveform;
   an asymmetrical biphasic waveform;
   a double monophasic waveform; and
   a monophasic waveform.
343. The system according to 342, wherein the electrical stimulator is configured to apply the one or more waveforms with a DC offset.
344. The system according to 342, wherein the DC offset is an applied voltage that is sufficient to compensate for each applied electrical stimulation pulse.
345. The system according to any one of 342-344, wherein each waveform is applied from different channels of the electrical stimulator.
346. The system according to any one of 342-345, wherein each waveform comprises a high frequency component and a low frequency component and wherein the high frequency component provides an analgesic effect on the skin and the low frequency component tunes the spinal cord neurons to achieve the required functional goals.
347. The system according to 346, wherein the high frequency component comprises a frequency of from 1 KHz to 25 KHz.
348. The system according to 347, wherein the high frequency component comprises a frequency of from 5 KHz to 15 KHz.
349. The system according to 347, wherein the high frequency component comprises a frequency of about 10 KHz.
350. The system according to any one of 345-349, wherein the low frequency component comprises a frequency of from 1 Hz to 500 Hz.
351. The system according to 350, wherein the low frequency component comprises a frequency of from 50 Hz to 250 Hz.
352. The system according to 350, wherein the low frequency component comprises a frequency of about 100 Hz.
353. The system according to any one of 281-352, wherein the electrical stimulator is configured to apply the electrical stimulation at a pulse frequency of 5 Hz or more.
354. The system according to any one of 281-352, wherein the electrical stimulator is configured to apply the electrical stimulation at a pulse frequency of 25 Hz or more.
355. The system according to any one of 281-352, wherein the electrical stimulator is configured to apply the electrical stimulation at a pulse frequency of about 30 Hz.
356. The system according to any one of 281-355, wherein the electrical stimulator is configured to apply electrical stimulation having a pulse amplitude of from 1 mA to 500 mA.
357. The system according to any one of 281-355, wherein the electrical stimulator is configured to apply electrical stimulation having a pulse amplitude of from 50 mA to 200 mA.
358. The system according to 357, wherein the electrical stimulator is configured to apply electrical stimulation having a pulse amplitude of about 100 mA.
359. The system according to any one of 281-358, wherein the electrical stimulator is configured to apply electrical stimulation having a DC offset amplitude of from 0.1 mA to 10 mA.
360. The system according to 359, wherein the electrical stimulator is configured to apply electrical stimulation having a DC offset amplitude of from 0.5 mA to 2.5 mA.
361. The system according to 360, wherein the electrical stimulator is configured to apply electrical stimulation having a DC offset amplitude of about 1.5 mA.
362. The system according to any one of 281-361, wherein the electrical stimulator is configured to apply a pulsed DC offset.
363. The system according to 362, wherein the electrical stimulator is configured to apply a continuously applied DC offset.
364. The system according to any one of 281-363, wherein the electrical stimulator is integrated into clothing.
365. The system according to any one of 281-364 wherein the electrical stimulator is integrated into a chair.
366. The system according to any one of 281-365, wherein the electrical stimulator is integrated into a wearable device.
367. The system according to 366, wherein the wearable device is a disposable device.
368. The system according to any one of 366-367, wherein the wearable device comprises a single use battery.
369. The system according to 368, wherein the wearable device comprises a reuseable rechargeable battery.
370. The system according to any one of 366-369, wherein the wearable device comprises a belt or harness worn spring and is configured to ensure hydrogel contact with the skin of the subject.
371. The system according to 370, wherein the springs of the wearable device are configured to provide mechanical and vibrotactile stimulation.
372. The system according to any one of 366-371, wherein the wearable device is configured to route wires under the clothing of the subject and to be connected to a spring based holder.
373. The system according to any one of 281-372, further comprising a mechanical stimulator configured to apply mechanical stimulation to the spine of a subject.
374. The system according to 373, wherein the mechanical stimulator is configured to apply mechanical stimulation to the spine of the subject simultaneously with the electrical stimulation.
375. The system according to 373, wherein the mechanical stimulator is configured to apply mechanical stimulation to the spine of the subject sequentially with the electrical stimulation.
376. The system according to any one of 281-375, further comprising a magnetic stimulator configured to apply magnetic stimulation to the spine of a subject.
377. The system according to 376, wherein the magnetic stimulator is configured to apply magnetic stimulation to the spine of the subject simultaneously with the electrical stimulation.
378. The system according to 377, wherein the magnetic stimulator is configured to apply magnetic stimulation to the spine of the subject sequentially with the electrical stimulation.
379. A system for neuromodulating the central nervous system of a subject having a spinal cord injury, the system comprising an electrical stimulator configured to apply electrical stimulation to the spinal cord of a subject acutely after the spinal cord injury in a manner sufficient to induce a plastic change in one or more of the brain and spinal cord.
380. The system according to 379, wherein the electrical stimulator is configured to apply electrical stimulation to the spinal cord of the subject in a manner sufficient to prevent aberrant connections in the brain and spinal cord of the subject.
381. The system according to 379, wherein the electrical stimulator is configured to apply electrical stimulation to the spinal cord of the subject in a manner sufficient to prevent aberrant connections in the brain and spinal cord of the subject during post-injury spinal shock.
382. The system according to any one of 379-381, wherein the electrical stimulator is configured to apply electrical stimulation to the spinal cord of the subject in a manner sufficient to reduce or prevent scar tissue formation at the site of the spinal cord injury.
383. The system according to any one of 379-382, wherein the electrical stimulator is configured to apply electrical stimulation to the spinal cord of the subject in a manner sufficient to increase blood flow to the site of the spinal cord injury.
384. The system according to any one of 379-382, wherein the electrical stimulator is configured to apply electrical stimulation to the spinal cord of the subject in a manner sufficient to increase blood flow to a site along the spinal cord that is above the spinal cord injury and/or to a site along the brain that is above the spinal cord injury.
385. The system according to any one of 379-382, wherein the electrical stimulator is configured to apply electrical stimulation to the spinal cord of the subject in a manner sufficient to increase blood flow to a site along the spinal cord that is below the spinal cord injury.
386. The system according to any one of 379-385, wherein the electrical stimulator is configured to apply electrical stimulation to the spinal cord of the subject in a manner sufficient to delay or prevent detrusor overactivity in the subject.
387. The system according to 386, wherein the electrical stimulator is configured to apply electrical stimulation to the spinal cord of the subject in a manner sufficient to delay or prevent detrusor overactivity in the subject during post-injury spinal shock.
388. The system according to any one of 379-387, wherein the electrical stimulator is configured to apply electrical stimulation to the spinal cord of the subject in a manner sufficient to reduce spasticity of the detrusor and urethral sphincter.
389. The system according to any one of 386-388, the electrical stimulator is configured to apply electrical stimulation to the spinal cord of the subject in a manner sufficient to increase voluntary control of the urethral sphincter in the subject to allow contraction and relaxation of the muscle based on whether the subject intends to store urine or void urine.
390. The system according to 389, wherein the subject is capable of one or more of storing urine in the bladder and voluntarily voiding the urine from the bladder during electrical stimulation.
391. The system according to 389, wherein the subject is capable of one or more of storing urine in the bladder and voluntarily voiding the urine from the bladder in the absence of active electrical stimulation.
392. The system according to any one of 386-391, wherein the subject is capable of one or more of voluntarily contracting the detrusor and simultaneously relaxing the urethral sphincter in the absence of active electrical stimulation.
393. The system according to any one of 386-392, wherein the electrical stimulator is configured to apply electrical stimulation to the spinal cord of the subject in a manner sufficient to increase sense by the subject of bladder fullness.
394. The system according to any one of 386-393, wherein the electrical stimulator is configured to apply electrical stimulation to the spinal cord of the subject in a manner sufficient to increase bladder capacity of the subject.
395. The system according to any one of 386-394, wherein th the electrical stimulator is configured to apply electrical stimulation to the spinal cord of the subject in a manner sufficient to facilitate voluntary delayed voiding contraction.
396. The system according to 395, wherein voiding contraction is delayed by an applied voluntary increase in urethral pressure by the subject.
397. The system according to 395, wherein the voluntary increase in urethral pressure is applied in a sustained manner.
398. The system according to 395, wherein the voluntary increase in urethral pressure is not applied in a spastic manner.
399. The system according to any one of 386-398, wherein the electrical stimulator is configured to apply electrical stimulation to the spinal cord of the subject in a manner sufficient to facilitate voluntary detrusor contraction.
400. The system according to 399, wherein the electrical stimulator is configured to apply electrical stimulation to the spinal cord of the subject in a manner sufficient to facilitate a decrease in urethral pressure in response to voluntary detrusor contraction.
401. The system according to any one of 386-400, wherein the frequency of voluntary voids increases in the absence of active stimulation.
402. The system according to any one of 386-401, wherein the volume of voluntary voids increases in the absence of active stimulation.
403. The system according to any one of 386-401, wherein the number of catheters used decreases in the absence of active stimulation.
404. The system according to any one of 379-403, wherein the electrical stimulator is configured to apply electrical stimulation to the spinal cord of the subject in a manner sufficient to increase one or more of voluntary initiation and voluntary completion of bowel movement by the subject.
405. The system according to any one of 379-403, wherein the electrical stimulator is configured to apply electrical stimulation to the spinal cord of the subject in a manner sufficient to increase sense by the subject of bowel fullness.
406. The system according to any one of 404-405, wherein the electrical stimulator is configured to apply electrical stimulation to the spinal cord of the subject in a manner sufficient to facilitate voluntary contractions of one or more of the anus, rectum and other bowel sections.
407. The system according to any one of 379-406, wherein the electrical stimulator is configured to apply electrical stimulation to the spinal cord of the subject in a manner sufficient to increase one or more voluntary sexual function by the subject.
408. The system according to 407, wherein the the electrical stimulator is configured to apply electrical stimulation to the spinal cord of the subject in a manner sufficient to facilitate voluntary generation of psychogenic erection by the subject.
409. The system according to 407, wherein the electrical stimulator is configured to apply electrical stimulation to the spinal cord of the subject in a manner sufficient to facilitate voluntary generation of reflex erection by the subject.
410. The system according to 407, wherein the electrical stimulator is configured to apply electrical stimulation to the spinal cord of the subject in a manner sufficient to facilitate voluntary ejaculation by the subject.
411. The system according to 407, wherein the electrical stimulator is configured to apply electrical stimulation to the spinal cord of the subject in a manner sufficient to facilitate performance of sexual intercourse by the subject.
412. The system according to 407, wherein the electrical stimulator is configured to apply electrical stimulation to the spinal cord of the subject in a manner sufficient to increase or improve sense of sexual function by the subject.
413. A method of neuromodulation in a subject for treating an accelerated aging condition caused by abnormal or delayed brain development in a subject, said method comprising of administering electrical neuromodulation to the spinal cord of the subject in a manner sufficient to slow down one or more aging milestones.
414. The method according to 413, wherein the subject suffers from non-traumatic brain damage.
415. The method according to 414, wherein the subject suffers from non-traumatic brain damage that occurred within about 2-3 years from birth.
416. The method according to any one of 413-415, wherein the subject is 5 years old or younger.
417. The method according to any one of 413-416, wherein the method comprises commencing treatment of the subject with the neuromodulation before the subject is 5 years old.
418. The method according to any one of 413-417, wherein the neuromodulation is administered to the subject at predetermined intervals for 1 year or more.
419. The method according to 418, wherein the neuromodulation is administered to the subject at predetermined intervals for 10 years or more.
420. The method according to 418, wherein the neuromodulation is administered to the subject at predetermined intervals for the duration of the subject's life.
421. The method according to any one of 413-420, wherein the neuromodulation comprises applying the electrical stimulation at a frequency and amplitude sufficient to decrease symptoms of aging.
422. The method according to any one of 413-421, wherein the neuromodulation comprises applying the electrical stimulation at a frequency and amplitude sufficient to extend life span of the subject.
423. The method according to any one of 413-422, wherein the neuromodulation comprises applying the electrical stimulation at a frequency and amplitude sufficient to maintain functionality and extending the age at which said subject is able to perform at their peak.
424. The method according to any one of 413-422, wherein the neuromodulation comprises applying the electrical stimulation in a manner sufficient to enable and learn a non-patterned, non-repetitive, stochastic motor response by the subject.
425. The method according to any one of 413-424, wherein the subject has or is diagnosed with cerebral palsy.
426. The method according to any one of 413-425, wherein the method comprises applying the electrical stimulation at a frequency and amplitude sufficient to reduce or prevent deterioration of walking capabilities associated with aging.
427. The method according to any one of 413-426, wherein the method comprises applying the electrical stimulation at a frequency and amplitude sufficient to reduce or prevent one or more of mental and physical fatigue associated with aging.
428. The method according to any one of 413-427, wherein the method comprises applying the electrical stimulation at a frequency and amplitude sufficient to reduce or prevent visual deficit associated with aging.
429. The method according to any one of 413-428, wherein the method comprises applying the electrical stimulation at a frequency and amplitude sufficient to reduce or prevent hearing deficit associated with aging.
430. The method according to any one of 413-429, wherein the method comprises applying the electrical stimulation at a frequency and amplitude sufficient to reduce or prevent cardiovascular disease or complications associated with aging.
431. The method according to any one of 413-430, wherein the method comprises applying the electrical stimulation at a frequency and amplitude sufficient to reduce or prevent one or more gastrointestinal indications or diseases associated with aging.
432. The method according to 431, wherein the method comprises applying the electrical stimulation at a frequency and amplitude sufficient to reduce or prevent one or more of constipation and fecal incontinence associated with aging.
433. The method according to any one of 413-432, wherein the method comprises applying the electrical stimulation at a frequency and amplitude sufficient to reduce or prevent speech impairment associated with aging.
434. The method according to any one of 413-433, wherein the method comprises applying the electrical stimulation at a frequency and amplitude sufficient to improve impairment of one or more of chewing and swallowing associated with aging.
435. The method according to any one of 413-434, wherein the method comprises applying the electrical stimulation at a frequency and amplitude sufficient to reduce or prevent pain during sitting or standing that is associated with aging.
436. The method according to any one of 413-435, wherein the method comprises applying the electrical stimulation at a frequency and amplitude sufficient to reduce or prevent scoliosis associated with aging.
437. The method according to any one of 413-436, wherein the method comprises applying the electrical stimulation at a frequency and amplitude sufficient to reduce or prevent the occurrence of seizures associated with aging.
438. The method according to any one of 413-437, wherein the method comprises applying the electrical stimulation at a frequency and amplitude sufficient to reduce or prevent genitourinary dysfunction associated with aging.
439. The method according to 438, wherein the genitourinary dysfunction comprises one or more of irritable bladder, bladder dysfunction, frequent urination, ureteral reflex, hypotonic enlarged bladder, frequent urinary tract infection and urinary incontinence.
440. The method according any one of 413-440, wherein the method comprises applying the electrical stimulation at a frequency and amplitude sufficient to reduce or prevent sexual dysfunction associated with aging.
441. The method according to 440, wherein the sexual dysfunction comprises one or more of sexual desire and voluntary movement during sexual activities.
442. The method according to any one of 413-440, wherein the method comprises applying the electrical stimulation at a frequency and amplitude sufficient to one or more of attenuate, arrest or reverse bone loss associated with aging.
443. The method according to 442, wherein the bone loss is in the lower extremities of the subject.
444. The method according to any one of 442-443, wherein the bone loss is associated with or caused by paralysis.
445. The method according to any one of 442-444, wherein the bone loss is associated with or caused by delayed development.
446. The method according to any one of 413-440, wherein the method comprises applying the electrical stimulation at a frequency and amplitude sufficient to one or more of attenuate, arrest or reverse osteoporosis associated with aging.
447. The method according to 446, wherein the osteoporosis is in the lower extremities of the subject.
448. The method according to any one of 446-447, wherein the osteoporosis is associated with or caused by paralysis.
449. The method according to any one of 446-448, wherein the osteoporosis is associated with or caused by delayed development.
450. The method according to any one of 413-449, wherein the method comprises applying the electrical stimulation at a frequency and amplitude sufficient to heal a bone fracture.
451. The method according to any one of 413-450, wherein the method comprises applying the electrical stimulation at a frequency and amplitude sufficient to regrow bone.
452. The method according to 451, wherein the bone is in need of regrowth due to a bone fracture.
453. The method according to any one of 446-452, wherein the bone fracture is the result of an accidental fall.
454. The method according to any one of 442-453, wherein the method comprises applying the electrical stimulation at a frequency and amplitude sufficient to change bone density as determined by CT scans or DEXA scans.
455. The method according to 454, wherein the bone density is increased by 5% or more as determined by CT scans or DEXA scans, such as by 10% or more, such as by 20%, such as by 30% or more, such as by 40% or more, such as by 50% or more, such as by 60% or more, such as by 70% or more, such as by 80% or more and including by 90% or more.
456. The method according to any one of 426-455, wherein applying the electrical stimulation at a frequency and amplitude sufficient to slow deterioration by the subject due to aging.
457. The method according to any one of 426-456, wherein the aging comprises aging with cerebral palsy.
458. The method according to any one of 413-457, wherein the neuromodulation is sufficient to increase development rate in subjects having cerebral palsy.
459. The method according to any one of 413-458, wherein the neuromodulation is sufficient to move the peak performance age of the subject to an older age.
460. The method according to any one of 413-459, wherein the neuromodulation is sufficient to move increase peak performance by the subject.
461. The method according to any one of 454-460, wherein the neuromodulation is sufficient to slow deterioration after the subject reaches peak performance.
462. The method according to any one of 413-461, wherein the subject exhibits one or more of:
   a) stiff muscles and exaggerated reflexes (spasticity);
   b) variations in muscle tone, such as being either too stiff or too floppy;
   c) stiff muscles with normal reflexes (rigidity);
   d) lack of balance and muscle coordination (ataxia);
   e) tremors or jerky involuntary movements;
   f) slow, writhing movements;
   g) favoring one side of the body, such as only reaching with one hand or dragging a leg while crawling;
   h) difficulty walking, such as walking on toes, a crouched gait, a scissors-like gait with knees crossing, a wide gait or an asymmetrical gait; and
   i) difficulty with fine motor skills, such as buttoning clothes or picking up utensils.
463. The method according to any one of 413-462, wherein the subject exhibits one or more of:
   a) delays in speech development;
   b) difficulty speaking;
   c) difficulty with sucking, chewing or eating; and
   d) excessive drooling or problems with swallowing.
464. The method according to any one of 413-463, wherein the subject exhibits one or more of:
   a) delays in reaching motor skills milestones, such as sitting up or crawling;
   b) learning difficulties;
   c) intellectual disabilities;
   d) delayed growth, resulting in smaller size than would be expected.
465. The method according to any one of 413-464, wherein the subject exhibits one or more of:
   a) seizures (epilepsy);
   b) difficulty hearing;
   c) problems with vision and abnormal eye movements;
   d) abnormal touch or pain sensations;
   e) bladder and bowel problems, including constipation and urinary incontinence; and
   f) mental health conditions, such as emotional disorders and behavioral problems.
466. The method according to any one of 413-465, wherein the subject is diagnosed as having spastic cerebral palsy.
467. The method according to 466, wherein the spastic cerebral palsy is characterized by one or more of spastic diplegia, spastic hemiplegia an spastic quadriplegia.
468. The method according to any one of 413-465, wherein the subject is diagnosed as having dyskinetic cerebral palsy.
469. The method according to 468, wherein the dyskinetic cerebral palsy is characterized by one or more of athetoid, choreoathetoid and dystonic.
470. The method according to any one of 413-465, wherein the subject is diagnosed as having ataxias cerebral palsy.
471. The method according to any one of 413-465, wherein the subject is diagnosed as having mixed cerebral palsy.
472. A system for neuromodulating the central nervous system of a subject for treating an accelerated aging condition caused by abnormal or delayed brain development, the system comprising an electrical stimulator configured to apply electrical stimulation to the spinal cord of a subject in a manner sufficient to to slow down one or more aging milestones.
473. The system according to 472, wherein the electrical stimulator is configured to apply electrical stimulation to the spinal cord of the subject at a frequency and amplitude sufficient to decrease symptoms of aging.
474. The system according to any one of 472-473, wherein the electrical stimulator is configured to apply electrical stimulation to the spinal cord of the subject at a frequency and amplitude sufficient to extend life span of the subject.
475. The system according to any one of 472-474, wherein the electrical stimulator is configured to apply electrical stimulation to the spinal cord of the subject at a frequency and amplitude sufficient to maintain functionality and extending the age at which said subject is able to perform at their peak.
476. The system according to any one of 472-475, wherein the electrical stimulator is configured to apply electrical stimulation to the spinal cord of the subject at a frequency and amplitude sufficient to enable and learn a non-patterned, non-repetitive, stochastic motor response by the subject.
477. The system according to any one of 472-476, wherein the electrical stimulator is configured to apply electrical stimulation to the spinal cord of the subject at a frequency and amplitude sufficient to reduce or prevent deterioration of walking capabilities associated with aging.
478. The system according to any one of 472-477, wherein the electrical stimulator is configured to apply electrical stimulation to the spinal cord of the subject at a frequency and amplitude sufficient to reduce or prevent one or more of mental and physical fatigue associated with aging.
479. The system according to any one of 472-478, wherein the electrical stimulator is configured to apply electrical stimulation to the spinal cord of the subject at a frequency and amplitude sufficient to reduce or prevent visual deficit associated with aging.
480. The system according to any one of 472-479, wherein the electrical stimulator is configured to apply electrical stimulation to the spinal cord of the subject at a frequency and amplitude sufficient to reduce or prevent hearing deficit associated with aging.
481. The system according to any one of 472-480, wherein the electrical stimulator is configured to apply electrical stimulation to the spinal cord of the subject at a frequency and amplitude sufficient to reduce or prevent cardiovascular disease or complications associated with aging.
482. The system according to any one of 472-481, wherein the electrical stimulator is configured to apply electrical stimulation to the spinal cord of the subject at a frequency and amplitude sufficient to reduce or prevent one or more gastrointestinal indications or diseases associated with aging.
483. The system according to any one of 472-482, wherein the electrical stimulator is configured to apply electrical stimulation to the spinal cord of the subject at a frequency and amplitude sufficient to reduce or prevent one or more of constipation and fecal incontinence associated with aging.
484. The system according to any one of 472-483, wherein the electrical stimulator is configured to apply electrical stimulation to the spinal cord of the subject at a frequency and amplitude sufficient to reduce or prevent speech impairment associated with aging.
485. The system according to any one of 472-484, wherein the electrical stimulator is configured to apply electrical stimulation to the spinal cord of the subject at a frequency and amplitude sufficient to improve impairment of one or more of chewing and swallowing associated with aging.
486. The system according to any one of 472-485, wherein the electrical stimulator is configured to apply electrical stimulation to the spinal cord of the subject at a frequency and amplitude sufficient to reduce or prevent pain during sitting or standing that is associated with aging.
487. The system according to any one of 472-486, wherein the electrical stimulator is configured to apply electrical stimulation to the spinal cord of the subject at a frequency and amplitude sufficient to reduce or prevent scoliosis associated with aging.
488. The system according to any one of 472-487, wherein the electrical stimulator is configured to apply electrical stimulation to the spinal cord of the subject at a frequency and amplitude sufficient to reduce or prevent the occurrence of seizures associated with aging.
489. The system according to any one of 472-488, wherein the electrical stimulator is configured to apply electrical stimulation to the spinal cord of the subject at a frequency and amplitude sufficient to reduce or prevent genitourinary dysfunction associated with aging.
490. The system according to 489, wherein the genitourinary dysfunction comprises one or more of irritable bladder, bladder dysfunction, frequent urination, ureteral reflex, hypotonic enlarged bladder, frequent urinary tract infection and urinary incontinence.
491. The system according to any one of 472-490, wherein the electrical stimulator is configured to apply electrical stimulation to the spinal cord of the subject at a frequency and amplitude sufficient to reduce or prevent sexual dysfunction associated with aging.
492. The system according to 491, wherein the sexual dysfunction comprises one or more of sexual desire and voluntary movement during sexual activities.
493. The system according to any one of 472-492, wherein the electrical stimulator is configured to apply electrical stimulation to the spinal cord of the subject at a frequency and amplitude sufficient to one or more of attenuate, arrest or reverse bone loss associated with aging.
494. The system according to 493, wherein the bone loss is in the lower extremities of the subject.
495. The system according to any one of 493-494, wherein the bone loss is associated with or caused by paralysis.
496. The system according to any one of 493-495, wherein the bone loss is associated with or caused by delayed development.
497. The system according to any one of 472-496, wherein the electrical stimulator is configured to apply electrical stimulation to the spinal cord of the subject at a frequency and amplitude sufficient to one or more of attenuate, arrest or reverse osteoporosis associated with aging.
498. The system according to 497, wherein the osteoporosis is in the lower extremities of the subject.
499. The system according to any one of 497-498, wherein the osteoporosis is associated with or caused by paralysis.
500. The system according to any one of 497-499, wherein the osteoporosis is associated with or caused by delayed development.
501. The system according to any one of 472-500, wherein the electrical stimulator is configured to apply electrical stimulation to the spinal cord of the subject at a frequency and amplitude sufficient to heal a bone fracture.
502. The system according to any one of 472-501, wherein the electrical stimulator is configured to apply electrical stimulation to the spinal cord of the subject at a frequency and amplitude sufficient to regrow bone.
503. The system according to 502, wherein the bone is in need of regrowth due to a bone fracture.
504. The system according to 503, wherein the bone fracture is the result of an accidental fall.
505. The system according to any one of 493-504, wherein the electrical stimulator is configured to apply electrical stimulation to the spinal cord of the subject at a frequency and amplitude sufficient to change bone density as determined by CT scans or DEXA scans.
506. The system according to 505, wherein the bone density is increased by 5% or more as determined by CT scans or DEXA scans, such as by 10% or more, such as by 20%, such as by 30% or more, such as by 40% or more, such as by 50% or more, such as by 60% or more, such as by 70% or more, such as by 80% or more and including by 90% or more.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of neuromodulating the central nervous system of a subject, the method comprising applying electrical stimulation to the spinal cord of a subject in a manner sufficient to maintain voluntary control of physical activity during:
when electrical stimulation is actively delivered;
wherein in the absence of electrical stimulation the subject is capable of maintaining voluntary control of physical activity due to the neuroplasticity induced in the brain and spinal cord by the neuromodulation.

2. The method according to claim 1, wherein applying the electrical stimulation is sufficient to integrate and reconnect the brain to the spinal cord.

3. The method according to any one of claims 1-2, wherein applying electrical stimulation to the spinal cord of the subject is sufficient to excite neurons in the brain and spinal cord.

4. The method according to any one of claims 1-3, wherein applying electrical stimulation to the spinal cord of the subject is sufficient to inhibit neurons in the brain and spinal cord.

5. The method according to any one of claims 1-4, wherein the electrical stimulation comprises applying at least one waveform selected from the group consisting of:
one or more of a trapezoidal monophasic waveform and a trapezoidal biphasic waveform;
one or more of a triangular monophasic waveform and triangular biphasic waveform;
an asymmetrical biphasic waveform;
a double monophasic waveform; and
a monophasic waveform.

6. The method according to claim 5, wherein the one or more applied waveforms further comprises a DC offset.

7. The method according to any one of claims 5-6, wherein each waveform comprises a high frequency component and a low frequency component and wherein the high frequency component provides an analgesic effect on the skin and the low frequency component tunes the spinal cord neurons to achieve the required functional goals.

8. A method of neuromodulation in a subject having delayed or abnormal brain development, the method comprising applying electrical stimulation to the spinal cord of the subject in a manner sufficient to induce neuroplasticity of the brain and spinal cord neural network of the subject.

9. The method according to claim 8, wherein the neuromodulation comprises applying the electrical stimulation at a frequency and amplitude sufficient to:
activate one or more of the sensory neurons and the interneurons of the spinal cord neural network; and
not directly activate the motor neurons of the spinal cord neural network.

10. The method according to claim 9, wherein voluntary muscle control comprises one or more of:
activating one or more muscle groups;
inhibiting activity by one or more muscle groups; and
having no impact on one or more muscle groups.

11. The method according to claim 8-10, wherein the neuromodulation comprises applying the electrical stimulation in a manner sufficient to enable and learn a non-patterned, non-repetitive, stochastic motor response by the subject.

12. A system for neuromodulating the central nervous system of a subject, the system comprising an electrical stimulator configured to apply electrical stimulation to the spinal cord of a subject in a manner sufficient to maintain voluntary control of physical activity during the electrical stimulation.

13. The system according to claim 12, wherein the electrical stimulator comprises one or more channels configured to apply at least one waveform selected from the group consisting of:
one or more a trapezoidal monophasic waveform and a trapezoidal biphasic waveform;
one or more of a triangular monophasic waveform and triangular biphasic waveform;
an asymmetrical biphasic waveform;
a double monophasic waveform; and
a monophasic waveform.

14. The system according to any one of claims 12-13, wherein the electrical stimulator is integrated into a wearable device.
